(19) United States
(12) Patent Application Publication
(10) Pub. No.: US 2010/0041657 A1
(43) Pub. Date: Feb. 18, 2010

(54) HALOALKYLSULFONE SUBSTITUTED COMPOUNDS USEFUL FOR TREATING OBESITY AND DIABETES

(75) Inventors: Preben Houlberg Olesen, Copenhagen (DK); Holger Claus Hansen, Vaerlose (DK); Lise Brown Christiansen, Lyngby (DK); Flemming Elmelund Nielsen, Virum (DK); Anders Klarskov Petersen, Naerum (DK)

Correspondence Address: High Point Pharmaceuticals, Inc., 4170 Mendenhall Oaks Pkwy, High Point, NC, 27265, US

(73) Assignee: NOVO NORDICK A/S, BAGSVAERD (DK)

(21) Appl. No.: 11/913,720

(22) PCT Filed: May 11, 2006
(86) PCT No.: PCT/EP2006/062121

§ 371 Date: Oct. 29, 2009

(30) Foreign Application Priority Data
May 11, 2005 (EP) 05103909.7
Dec. 22, 2005 (EP) 05112773.6

(51) Int. Cl. A61K 31/5375; C07C 233/64; A61K 31/165; C07D 333/38; A61K 31/381; C07D 295/00; C07C 69/76; A61K 31/24; C07C 311/15; A61K 31/18; A61P 3/04; A61P 3/10

(52) U.S. Cl. 514/237.8; 564/174; 514/622; 564/162; 514/618; 564/172; 549/61; 514/447; 544/159; 560/59; 514/539; 564/85; 514/603

(57) ABSTRACT

The present invention relates to compounds of formula I, in which at least one of R5, R6 and R7 is SR12, S(O)R12 or S(O)2R12 group where R12 is a C1-6haloalkyl group, that act, as chemical uncouplers. Compounds of the invention are useful in the treatment, including prevention, of obesity, diabetes and a number of diseases or conditions associated therewith.

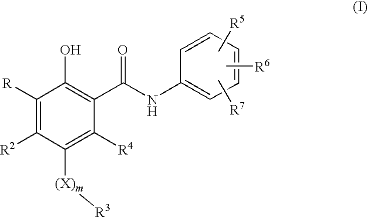

### FIELD OF THE INVENTION

The invention relates to trifluoromethylsulfone derivatives that have been found to be particularly interesting as chemical uncouplers.

### BACKGROUND OF THE INVENTION

Obesity is a well-known risk factor for the development of many very common diseases such as atherosclerosis, hypertension, type 2 diabetes (non-insulin dependent diabetes mellitus (NIDDM)), dyslipidemia, coronary heart disease, and osteoarthritis and various malignancies. It also causes considerable problems through reduced motility and decreased quality of life. The incidence of obese people and thereby also these diseases is increasing throughout the entire industrialised world.

The term obesity implies an excess of adipose tissue. In this context obesity is best viewed as any degree of excess adiposity that imparts a health risk. The cut off between normal and obese individuals can only be approximated, but the health risk imparted by the obesity is probably a continuum with increasing adiposity. In the context of the present invention, individuals with a body mass index (BMI=body weight in kilograms divided by the square of the height in meters) above 25 are to be regarded as obese

Even mild obesity increases the risk for premature death and conditions such as diabetes, dyslipidemia, hypertension, atherosclerosis, gallbladder disease and certain types of cancer. In the industrialised western world the prevalence of obesity has increased significantly in the past few decades. Because of the high prevalence of obesity and its health consequences, its prevention and treatment should be a high public health priority.

Except for exercise, diet and food restriction, which is not feasible for a vast number of patients, no convincing treatment for reducing body weight effectively and acceptably currently exist. However, not only in view of the considerable problems directly related to obesity as described above, but also due to the important effect of obesity as a risk factor in serious and even mortal and common diseases, it is important to find pharmaceutical compounds which are useful in prevention and/or treatment of obesity.

When energy intake exceeds expenditure, the excess calories are stored predominately in adipose tissue, and if this net positive balance is prolonged, obesity results, i.e. there are two components to weight balance, and an abnormality on either side (intake or expenditure) can lead to obesity. This process may be counteracted by increasing the energy expenditure (for instance via exercise) or decreasing the energy intake (for instance by dieting). Pharmacological treatment available up to date only consists of Sibutramine (acting via serotonergic mechanisms, Abbott) and Orlistat (reducing fat uptake from the gut, Roche Pharm) neither reducing body weight effectively nor acceptably. There is therefore a need for pharmaceutical compounds which may be useful in prevention and/or treatment of obesity, for instance by increasing the energy expenditure or decreasing the energy intake.

One way of increasing energy expenditure is by increasing the metabolic rate. Oxidative phosphorylation in mitochondria, the energy from glucose metabolism and free fatty acids oxidation is used to drive the phosphorylation of ADP to ATP. When NADH and FADH₂ formed in the TCA cycle are oxidised back to NAD⁺ and FAD respectively, protons are pumped out of the mitochondrial matrix. The resulting pH gradient (matrix pH-8 and outside pH˜7) and potential (˜−170 mV, inside negative) across the inner mitochondrial membrane constitute the electrochemical proton gradient. As the effect of a one-unit pH difference corresponds to a potential of 61.5 mV, the electrochemical proton gradient exerts a protonmotive force of roughly ˜230 mV, which is the driving force for the mitochondrial ATP synthesis.

When the ATP consumption thus increases, the cells respond by increasing the ATP synthesis and consequently the inward flux of protons through the ATP synthase, the enzyme responsible for ATP synthesis and thereby the metabolic rate is increased. Chemical uncouplers are compounds, which can transport protons across membranes, and when protons are transported across the inner mitochondrial membrane, the ATP synthase is bypassed. At the (alkaline) matrix side the proton is released and the deprotonated uncoupler returns to the inter-membrane space where it picks up another proton. The cycling of the uncoupler (or ATP synthesis) and the resulting proton transport leads to an increased outward pumping of protons through an increased oxidation of NADH and FADH₂ by the respiration chain. The NADH concentration in the matrix will consequently drop. Since NADH feed-back inhibits three steps in the TCA cycle (NADH is the main regulator of the TCA cycle), the flux through the TCA cycle will increase. Hence, the metabolic rate will increase.

Compounds, such as chemical uncouplers, which act by increasing the metabolic rate may thus be useful for treating obesity, but also for treating other conditions such as atherosclerosis, hypertension, diabetes, especially type 2 diabetes (NIDDM (non-insulin dependent diabetes mellitus)), dyslipidemia, coronary heart disease, gallbladder disease, osteoarthritis and various types of cancer such as endometrial, breast, prostate and colon cancers and the risk for premature death as well as other conditions, such as diseases and disorders, which conditions are improved by a reduced mitochondrial potential.

Furthermore, chemical uncouplers may reduce reactive oxygen species (ROS) that are assumed (De Grey et al, Eur J. Biochem 269, 1995 ff (2002)) to be involved in the aging process, in damage of heart tissue as well as neuronal tissue. It is therefore also possible that conditions affected by ROS may be reversed or halted by intervention by chemical uncouplers. Examples of such conditions include diabetic microvascular diseases in the retina, renal glomerulus and peripheral nerves cell.

The best known chemical uncoupler is 2,4-dinitrophenol (DNP), which has been shown to increase energy expenditure in humans as well as animals. The side effects at higher doses include increased perspiration, vasodilatation, skin rashes, cataracts, neuritis and even death. Two fatalities amongst the first 100.000 persons treated with DNP, and the fact that the lowest dose which could be lethal was only twice the average dose giving a desired 50% increase in basal metabolic rate, giving a very narrow safety window, combined with other factors led to the removal of DNP from the market. Since then nobody has attempted to develop or market uncouplers for the treatment of obesity.

DNP is the best known chemical uncoupler; but many other compounds are known to induce uncoupling. DNP derivatives such as 4,6-dinitro-o-cresol (Victoria Yellow) and 2,4-dinitro-1-naphtol (Martius Yellow) as well as structurally unrelated compounds such as 2,6-di-t-butyl-4-(2′,2′-dicyanovinyl)phenol) (SF6847) (also known as 2-(3,5-di-tert-butyl-4-hydroxybenzylidene)-malononitrile), carbonylcyanide m-chlorophenylhydrazone (CCCP) and carbonylcyanide ptrifluoromethoxy-phenylhydrazone (FCCP) (Miyoshi H et al. Quantitative relationship between protenophoric and uncoupling activities of analogs of SF6847 (2,6-di-t-butyl-4-(2′,2′-dicyanovinyl)phenol), Biochimica et Biophysica Acta 891, 293-299 (1987)) are uncouplers.

Another class of chemical uncouplers is the salicylanilides of which S-13 is the most potent compound discovered so far (Terada H et al. Structural Requirements of Salicylanilides for Uncoupling Activity in Mitochondria Quantitative Analysis of Structure-Uncoupling Relationships, Biochimica et Biophysica Acta 936, 504-512 (1988)).

WO00/06143 to Texas Pharmaceuticals Inc. relates to a method for inducing intracellular hyperthermia comprising a step of administering a mitochondrial uncoupling agent, such as 2,4-dinitrophenol.

U.S. Pat. No. 4,673,691 to Bachynsky relates to the use of 2,4-dinitrophenol for treating obesity.

Various salisylic anilide derivatives have been disclosed in the literature. As an example, U.S. Pat. No. 4,025,647 discloses compounds of the formula

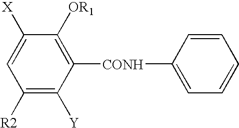

wherein R1 may be hydrogen, X is secondary or tertiary alkyl, R2 alkanoyl, phenylsulfinyl, phenylsulfonyl, etc, and Y is hydrogen or methyl. The compounds have anthelmintic activity, especially against liver fluke.

EP 322823 discloses electrophotographic photoreceptors with the following formula

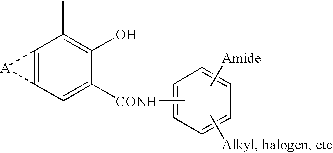

wherein A is a group of atoms necessary to condense the benzene ring with another ring.

WO 01/44172 discloses compounds of the formula

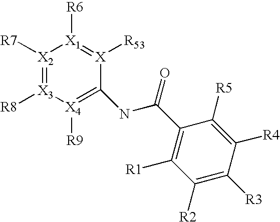

wherein all X's may be carbon, R1 may be hydroxyl, R2-R5 may be optionally substituted aryl heteroaryl, alkylaryl, alkyl, ester, amide, etc. The compounds are inhibitors of serine proteases, urokinase, Factor Xa, Factor VIIa and have utility as anticancer agents and as anticoagulants. R7 is amidine or guadinyl for all compounds specifically disclosed in this application.

WO 01/96944 discloses compounds of the formula

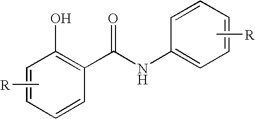

wherein R represent 0-4 substituents selected from alkyl, aryl, aralkyl, etc. The compounds are useful as components in colour photothermographic films. None of the specifically disclosed compounds have a branched alkyl or phenyl as substituent in the left-most phenyl ring.

WO 01/82924 discloses compounds of the formula

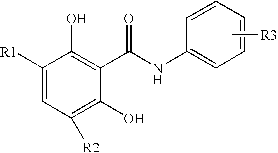

wherein R1-3 represents hydrogen, alkyl, halo, alkoxy, etc. The compounds are phosphate transport inhibitors.

### SUMMARY OF THE INVENTION

The invention provides a compound according to formula I

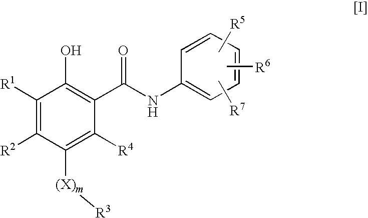

wherein
m is 0, such that R³ is linked directly to the benzene ring by a bond, or
m is 1 or 2;

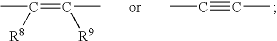

X, when present, represents a group
R¹ represents hydrogen or halogen; or R¹ represents C₁₋₈alkyl, C₁₋₆alkenyl, C₁₋₆alkynyl, C₃₋₈cycloalkyl, C₄₋₈cycloalkenyl or phenyl, all of which may optionally be further substituted by halogen, C₁₋₈alkyl, C₃₋₈cycloalkyl, C₄₋₈cycloalkenyl or phenyl; or R¹ represents bicyclo-C₄₋₁₀-alkyl or tricyclo-C₄₋₁₀-alkyl; wherein said C₃₋₈cycloalkyl, bicyclo-C₄₋₁₀alkyl, tricyclo-C₄₋₁₀-alkyl or phenyl are optionally substituted with one or more substituents selected among hydroxy, cyano, nitro, C₁₋₆alkyl, C₁₋₆alkenyl, C₁₋₆alkynyl, C₃₋₈-cycloalkyl, C₄₋₈cycloalkenyl, C₁₋₆alkoxy, C₁₋₆ haloalkoxy and C₁₋₆haloalkyl;
R² represents hydrogen, halogen, C₁₋₆alkyl, C₁₋₆alkenyl, C₁₋₆alkynyl, C₃₋₈-cycloalkyl, C₄₋₈cycloalkenyl or C₁₋₆alkoxy;
R⁴ represents, hydrogen, halogen, hydroxy, C₁₋₆alkyl, C₁₋₆alkenyl, C₁₋₆alkynyl, C₃₋₈-cycloalkyl, C₄₋₈cycloalkenyl or C₁₋₆alkoxy;
R⁸ and R⁹ independently represent, hydrogen, C₁₋₆alkyl, C₁₋₆alkenyl, C₁₋₆alkynyl, C₃₋₈-cycloalkyl, C₄₋₈cycloalkenyl or C₁₋₆alkoxy;
or
R¹ and R² together with the benzene ring, or, with the proviso that m is 0, R² and R³ together with the benzene ring or R³ and R⁴ together with the benzene ring form a 9-11-membered bicyclic ring system which may be fully conjugated or partly saturated, and which may optionally be substituted with one or more substituents selected among halogen, hydroxy, nitro, cyano, C₁₋₆alkyl, C₁₋₆-alkenyl, C₁₋₆alkynyl, C₃₋₈cycloalkyl, C₄₋₈cycloalkenyl, C₁₋₆alkoxy, C₁₋₆haloalkoxy and C₁₋₆-haloalkyl;
at least one substituent among R⁵, R⁶ and R⁷ represents —SR¹², —S(O)R¹² or —S(O)₂R¹², and the remaining substituents among R⁵, R⁶ and R⁷ independently represent hydrogen, nitro, cyano, halogen, C₁₋₆alkyl, C₁₋₆alkenyl, C₁₋₆alkynyl, C₃₋₈cycloalkyl, C₄₋₈cycloalkenyl, C₁₋₆ haloalkyl, —OR¹⁰, —NR¹⁰R¹¹, —C(O)OR¹⁰, —COR¹⁰, —C(O)NR¹⁰R¹¹, —SH, —S(O)₂OR¹⁰, —S(O)₂NR¹⁰R¹¹, —SR¹¹, —S(O)R¹¹, —S(O)₂R¹¹, aryl or heteroaryl; wherein the latter aryl or heteroaryl may optionally be substituted with one or more substituents selected among C₁₋₆alkyl, halogen, hydroxy and phenyl;
R¹⁰ and R¹¹ independently represent hydrogen, C₁₋₆alkyl, C₁₋₆alkenyl, C₁₋₆alkynyl, C₃₋₈-cycloalkyl, C₄₋₈cycloalkenyl, C₁₋₆haloalkyl or C₁₋₆haloalkoxy;
R¹² represents C₁₋₆haloalkyl;
R³ represents hydrogen, amino, nitro, cyano, halogen, C₁₋₆alkyl, C₁₋₆alkenyl, C₁₋₆alkynyl, C₁₋₆-haloalkyl, arylC₁₋₆alkyl, arylC₁₋₆alkenyl, arylC₁₋₆alkynyl, heteroarylC₁₋₆alkyl, heteroarylC₁₋₆-alkenyl, heteroarylC₁₋₆alkynyl, C₃₋₈cycloalkyl, —OR¹⁷, —NR¹⁷R¹⁸, C₁₋₆haloalkyl, —C(O)OR¹⁷, —COR¹⁷, —C(O)NR¹⁷R¹⁸, —SH, —S(O)₂OR¹⁷, —S(O)₂NR¹⁷R¹⁸, —SR¹⁷, —S(O)R¹⁷, —S(O)₂R¹⁷, —NH—COR¹⁷ or —NH—S(O)₂R¹⁷; or R³ represents aryl or heteroaryl which may optionally be substituted with one, two, three or four substituents independently selected among halogen, cyano, nitro, C₁₋₆alkyl, C₁₋₆alkenyl, C₁₋₆alkynyl, C₃₋₈cycloalkyl, C₄₋₈cycloalkenyl, C₁₋₆alkoxy, C₁₋₆haloalkoxy, C₁₋₆haloalkyl, C₁₋₆hydroxyalkyl, C₁₋₆alkylaryl, hydroxy, —(CH₂)ᵣOR¹³, —SH, —S(O)ₚR¹³, —S(O)ₚN(R¹³)(R¹⁴), —C(O)OR¹³, —OC(O)R¹³, —C(O)R¹³, —C(O)N(R¹³)(R¹⁴), (CH₂)ᵣN(R¹³)C(O)R¹⁴, —B(OR¹³)(OR¹⁴), —(CH₂)ᵣN(R¹³)(R¹⁴) and phenyl; wherein the latter phenyl is optionally substituted with one or more substituents selected among cyano, nitro, C₁₋₆alkyl, halogen, C₁₋₆haloalkyl, C₁₋₆hydroxyalkyl, C₁₋₆haloalkoxy, —OR¹⁵—, —S(O)ₛR¹⁵, —C(O)OR¹⁵, —OC(O)R¹⁵, —C(O)R¹⁵, —C(O)N(R¹⁵)(R¹⁶), —N(R¹⁵)(R¹⁶), —(CH₂)ₛN(R¹⁵)C(O)R¹⁶, —B(OR¹⁵)(OR¹⁶), —(CH₂)ₜOR¹⁵ and —(CH₂)ₜN(R¹⁵)(R¹⁶);
R¹³, R¹⁴, R¹⁵, R¹⁶ and R¹⁸ independently represent hydrogen, C₁₋₆alkyl, C₁₋₆alkenyl, C₁₋₆alkynyl, C₃₋₈cycloalkyl, C₄₋₈cycloalkenyl, C₁₋₆haloalkyl, C₁₋₆hydroxyalkyl, C₁₋₆-aminoalkyl or phenyl, the latter phenyl optionally being substituted with one or more substituents selected among halogen, cyano, C₁₋₆alkyl, C₁₋₆alkenyl, C₁₋₆alkynyl, C₃₋₈cycloalkyl, C₄₋₈cycloalkenyl, C₁₋₆haloalkyl, C₁₋₆ haloalkoxy and C₁₋₆hydroxyalkyl;
R¹⁷ represent hydrogen, C₁₋₆alkyl, C₁₋₆alkenyl, C₁₋₆alkynyl, C₃₋₈cycloalkyl, C₄₋₈cycloalkenyl, C₁₋₆haloalkyl, C₁₋₆hydroxyalkyl, C₁₋₆-aminoalkyl, arylC₁₋₆alkyl or phenyl, the latter phenyl optionally being substituted with one or more substituents selected among halogen, cyano, C₁₋₆alkyl, C₁₋₆alkenyl, C₁₋₆alkynyl, C₃₋₈cycloalkyl, C₄₋₈cycloalkenyl, C₁₋₆haloalkyl, C₁₋₆haloalkoxy and C₁₋₆hydroxyalkyl;
or one or more of the substituent pairs R¹⁰ and R¹¹, R¹³ and R¹⁴, R¹⁵ and R¹⁶, and R¹⁷ and R¹⁸, when attached to the same nitrogen atom, together with the nitrogen atom form a saturated or unsaturated carbocyclic or heterocyclic 3-8-membered ring, optionally substituted with one or more C₁₋₆alkyl substituents;
p and s, independently of each other, are 0, 1 or 2;
r and t, independently of each other, are 0, 1, 2 or 3;
and pharmaceutically acceptable salts, solvates and prodrugs thereof.

Further aspects of the present invention provide:

a pharmaceutical composition comprising a compound according to the invention;
the use of a compound of the invention in the manufacture of a medicament for the treatment of diseases as disclosed herein; and
a method for the treatment of diseases as disclosed herein, comprising administering an effective amount (i.e. therapeutically effective amount) of a compound of the invention or a pharmaceutical composition of the invention, optionally in combination with one or more additional therapeutically active compounds as disclosed herein.

### DEFINITIONS

In the present context, the term “alkyl” is intended to indicate a straight- or branched-chain, saturated monovalent hydrocarbon radical having from one to twelve carbon atoms, also denoted C₁₋₁₂-alkyl. Typical alkyl groups are alkyl groups with from one to eight, such as from one to six, carbon atoms, also denoted C₁₋₈-alkyl and C₁₋₆-alkyl, respectively. Typical C₁₋₆-alkyl groups include, but are not limited to, methyl, ethyl, n-propyl, isopropyl, n-butyl, sec-butyl, isobutyl, tert-butyl, n-pentyl, 2-methylbutyl, 3-methylbutyl, 4-methylpentyl, n-pentyl, n-hexyl, 1,1-dimethylpropyl, 1,2-dimethylpropyl, 2,2-dimethylpropyl(neopentyl), 1,2,2-trimethylpropyl, 1,1,2,2-tetramethylpropyl, 1,1,3,3-tetramethyl-butyl and the like, while typical C₁₋₈-alkyl groups include the same groups as well as alkyl groups having seven or eight carbon atoms, such as heptyl, octyl, 2,2-dimethylhexyl and the like. The term “C₁₋₆-alkyl” as used herein also includes secondary C₃₋₆-alkyl and tertiary C₄₋₆-alkyl. The term “C₁₋₈-alkyl” as used herein also includes secondary C₃₋₈-alkyl and tertiary C₄₋₈-alkyl. The term “C₁₋₁₂-alkyl” as used herein also includes secondary C₃₋₁₂-alkyl and tertiary C₄₋₁₂-alkyl.

In the present context, the term “alkenyl” is intended to indicate a straight- or branched-chain, monovalent hydrocarbon radical having from two to six carbon atoms and at least one carbon-carbon double bond, for example C₃₋₅-alkenyl. Typical C₃₋₅-alkenyl groups include vinyl, alkyl, 1-propenyl, and the like. The term “conjugated alkenyl” as used herein, alone or in combination, refers to an alkenyl having at least two carbon-carbon double bonds that are separated by a carbon-carbon single bond, such as, for instance, 1,3-butadien-1-yl.

In the present context, the term “alkynyl” is intended to indicate a straight- or branched-chain, monovalent hydrocarbon radical having from two to six carbon atoms and at least one carbon-carbon triple bond, and optionally one or more carbon-carbon double bonds. Examples include ethynyl, propynyl and 3,4-pentadien-1-ynyl.

The terms “bicycloalkyl” and “tricycloalkyl” indicate fully saturated bicyclic and tricyclic structures, respectively. Examples include bicyclo[2.2.2]oct-1-yl, bicyclo[3.3.1]non-1-yl, 1-adamantyl and 2-adamantyl;

The term “halogen” is intended to indicate a substituent derived from an element in the seventh main group of the periodic system, which includes fluorine (giving rise to fluoro, F), chlorine (giving rise to chloro, Cl), bromine (giving rise to bromo, Br) and iodine (giving rise to iodo, I).

In the present context, the term “aryl” is intended to indicate a carbocyclic aromatic ring radical which may optionally be fused to another aromatic or non-aromatic ring. Typical aryl groups include phenyl, biphenylyl, indenyl, fluorenyl (1-fluorenyl, 2-fluorenyl, 3-fluorenyl or 4-fluorenyl), naphthyl (1-naphthyl or 2-naphthyl), anthracenyl (1-anthracenyl or 2-anthracenyl), 1,2,3,4-tetrahydro-quinolinyl, 1,2,3,4-tetrahydro-naphthyl, and the like.

The term “heteroaryl”, as used herein, alone or in combination, refers to:

an aromatic ring radical having, for instance, from 5 to 7 member atoms; or
a fused aromatic ring system radical having, for instance, from 7 to 18 member atoms, and wherein at least one ring is aromatic;
and containing one or more heteroatoms selected from nitrogen, oxygen and sulfur; wherein N-oxides and sulfur monoxides and sulfur dioxides are permissible heteroaromatic substitutions. Examples include furanyl, thienyl, thiophenyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, quinolinyl, isoquinolinyl, benzofuranyl, benzothiophenyl, indolyl and indazolyl, thienyl (2-thienyl or 3-thienyl), furanyl (2-furanyl or 3-furanyl), indolyl, oxadiazolyl, isoxazolyl, thiadiazolyl, oxatriazolyl, thiatriazolyl, quinazolinyl, fluorenyl, xanthenyl, isoindanyl, benzhydryl, acridinyl, thiazolyl, pyrrolyl (1-pyrrolyl, 2-pyrrolyl or 3-pyrrolyl), pyrazolyl (1-pyrazolyl, 3-pyrazolyl, 4-pyrazolyl or 5-pyrazolyl), imidazolyl (1-imidazolyl, 2-imidazolyl, 4-imidazolyl or 5-imidazolyl), triazolyl (1,2,3-triazol-1-yl, 1,2,3-triazol-4-yl 1,2,3-triazol-5-yl, 1,2,4-triazol-3-yl or 1,2,4-triazol-5-yl), oxazolyl (2-oxazolyl, 4-oxazolyl or 5-oxazolyl), isoxazolyl (isoxazo-3-yl, isoxazo-4-yl or isoxaz-5-yl), isothiazolyl(isothiazo-3-yl, isothiazo-4-yl or isothiaz-5-yl)thiazolyl (2-thiazolyl, 4-thiazolyl or 5-thiazolyl), pyridinyl (2-pyridinyl, 3-pyridinyl or 4-pyridinyl), pyrimidinyl (2-pyrimidinyl, 4-pyrimidinyl, 5-pyrimidinyl or 6-pyrimidinyl), pyrazinyl, pyridazinyl (3-pyridazinyl, 4-pyridazinyl or 5-pyridazinyl), quinolinyl (2-quinolinyl, 3-quinolinyl, 4-quinolinyl, 5-quinolinyl, 6-quinolinyl, 7-quinolinyl or 8-quinolinyl), isoquinolinyl (1-isoquinolinyl, 3-isoquinolinyl, 4-isoquinolinyl, 5-isoquinolinyl, 6-isoquinolinyl, 7-isoquinolinyl or 8-isoquinolinyl), benzo[b]furanyl (2-benzo[b]furanyl, 3-benzo[b]furanyl, 4-benzo[b]furanyl, 5-benzo[b]furanyl, 6-benzo[b]furanyl or 7-benzo[b]furanyl), 2,3-dihydrobenzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl) or 7-(2,3-dihydro-benzo[b]furanyl)), benzo[b]thiophenyl(benzo[b]thiophen-2-yl, benzo[b]thiophen-3-yl, benzo[b]thiophen-4-yl, benzo[b]thiophen-5-yl, benzo[b]thiophen-6-yl or benzo[b]thiophen-7-yl), 2,3-dihydro-benzo[b]thiophenyl (2,3-dihydro-benzo[b]thiophen-2-yl, 2,3-dihydrobenzo[b]thiophen-3-yl, 2,3-dihydro-benzo[b]thiophen-4-yl, 2,3-dihydro-benzo[b]thiophen-5-yl, 2,3-dihydro-benzo[b]thiophen-6-yl or 2,3-dihydro-benzo[b]thiophen-7-yl), indolyl (1-indolyl, 2-indolyl, 3-indolyl, 4-indolyl, 5-indolyl, 6-indolyl or 7-indolyl), indazolyl (1-indazolyl, 3-indazolyl, 4-indazolyl, 5-indazolyl, 6-indazolyl or 7-indazolyl), benzimidazolyl (1-benzimidazolyl, 2-benzimidazolyl, 4-benzimidazolyl, 5-benzimidazolyl, 6-benzimidazolyl, 7-benzimidazolyl or 8-benzimidazolyl), benzoxazolyl (2-benzoxazolyl, 3-benzoxazolyl, 4-benzoxazolyl, 5-benzoxazolyl, 6-benzoxazolyl or 7-benzoxazolyl), benzothiazolyl (2-benzothiazolyl, 4-benzothiazolyl, 5-benzothiazolyl, 6-benzothiazolyl or 7-benzothiazolyl), carbazolyl (1-carbazolyl, 2-carbazolyl, 3-carbazolyl or 4-carbazolyl), 5H-dibenz[b,f]azepinyl (5H-dibenz[b,f]azepin-1-yl, 5H-dibenz[b,f]azepin-2-yl, 5H-dibenz[b,f]azepin-3-yl, 5H-dibenz[b,f]azepin-4-yl or 5H-dibenz[b,f]azepin-5-yl), 10,11-dihydro-5H-dibenz[b,f]azepinyl (10,11-dihydro-5H-dibenz[b,f]azepin-1-yl, 10,11-dihydro-5H-dibenz[b,f]azepin-2-yl, 10,11-dihydro-5H-dibenz[b,f]azepin-3-yl, 10,11-dihydro-5H-dibenz[b,f]azepin-4-yl or 10,11-dihydro-5H-dibenz[b,f]azepin-5-yl), benzo[1,3]dioxole (2-benzo[1,3]dioxole, 4-benzo[1,3]dioxole, 5-benzo[1,3]dioxole, 6-benzo[1,3]dioxole or 7-benzo[1,3]dioxole), and tetrazolyl (5-tetrazolyl or N-tetrazolyl).

The term “bicyclic ring system” as used herein, alone or in combination, refers to a carbocyclic or heterocyclic ring fused to another carbocyclic or heterocyclic ring, the two rings having two atoms in common. Typical fused ring systems include, but are not limited to, napthalene, quinoline, isoquinoline, indole, isoindole, tetralin (1,2,3,4-tetrahydronaphthalene), indane, 2,3-dihydro-benzofuran, 2,3-dihydro-benzo[b]thiophen, chroman and thiochroman.

In the present context the term “cycloalkyl” is intended to indicate a cyclic saturated monovalent hydrocarbon radical having 3, 4, 5, 6, 7 or 8 ring carbon atoms. Examples hereof include cyclopropyl, cyclobutyl, cyclopentyl and cyclohexyl.

In the present context the term “cycloalkenyl” is intended to indicate a cyclic unsaturated monovalent hydrocarbon radical having 4, 5, 6, 7 or 8 ring carbon atoms. Examples hereof include cyclobutenyl, cyclopentenyl and cyclohexenyl.

In the present context, the term “alkoxy” is intended to indicate a radical of the formula —OR′, wherein R′ represents alkyl as indicated above.

The term “haloalkoxy” is intended to indicate an alkoxy as defined above substituted with one or more halogen substituents as defined above, e.g. fluoro, chloro, bromo or iodo.

The term “nitro” designates the radical —NO₂.

The term “cyano” designates the radical —CN.

In the present context, the term “haloalkyl” is intended to indicate an alkyl as defined above substituted with one or more halogen substituents as defined above. Examples include trihalomethyl, such as trifluoromethyl and trichloromethyl; further examples include trihaloethyl, such as 2,2,2-trifluoro-1-ethyl and 2,2,2-trichloro-1-ethyl.

In the present context, the term “hydroxyalkyl” is intended to indicate an alkyl as defined above substituted with one or more hydroxy groups. Examples include hydroxymethyl, 1-hydroxy-1-ethyl and 2-hydroxy-1-ethyl.

In the present context, the substituent designation S(O)ₙR^{x} refers to —SR^{x}, —S(O)R^{x} or —S(O)₂R^{x};

As used herein, the term “solvate” refers to a complex of defined stoichiometry formed by a solute (in casu, a compound according to the present invention) and a solvent. Relevant solvents include, by way of example, water, ethanol and acetic acid.

As used herein, the term “prodrug” includes derivatives of compounds of the invention such as biohydrolyzable amides and biohydrolyzable esters thereof, and also encompasses:

a) compounds in which the biohydrolyzable functionality in such a prodrug is encompassed in the compound according to the present invention; and
b) compounds which may be oxidized or reduced biologically at a given functional group to yield drug substances according to the present invention.

Examples of the latter type of functional group include 1,4-dihydropyridine, N-alkylcarbonyl-1,4-dihydropyridine, 1,4-cyclohexadiene, tert-butyl and the like.

In the present context, the term “pharmaceutically acceptable salt” is intended to indicate a salt which is not harmful to the patient. Such salts include pharmaceutically acceptable acid addition salts as well as pharmaceutically acceptable metal salts, ammonium salts and alkylated ammonium salts. Acid addition salts include salts of inorganic acids as well as organic acids. Representative examples of suitable inorganic acids include hydrochloric, hydrobromic, hydroiodic, phosphoric, sulfuric and nitric acid, and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene-salicylic, ethanedisulfonic, gluconic, citraconic, aspartic, stearic, palmitic, EDTA, glycolic, p-aminobenzoic, glutamic, benzenesulfonic and p-toluenesulfonic acid, and the like. Further examples of pharmaceutically acceptable inorganic or organic acid addition salts include the pharmaceutically acceptable salts listed in J. Pharm. Sci. 1977, 66, 2, the contents of which are incorporated herein by reference. Examples of metal salts include lithium, sodium, potassium and magnesium salts, and the like. Examples of ammonium and alkylated ammonium salts include ammonium, methylammonium, dimethylammonium, trimethylammonium, ethylammonium, hydroxyethylammonium, diethylammonium, butylammonium and tetramethylammonium salts, and the like.

The term “therapeutically effective amount” of a compound as used herein refers to an amount sufficient to cure, alleviate or partially arrest the clinical manifestations of a given disease or disorder and its complications. An amount adequate to accomplish this is defined as a “therapeutically effective amount”. The amount that is effective for a particular therapeutic purpose will depend on the severity of the disease or injury as well as on the weight and general state of the subject. It will be understood that determination of an appropriate dosage may be achieved, using routine experimentation, by constructing a matrix of values and testing different points in the matrix, all of which is within the ordinary skills of a trained physician or veterinary.

The terms “treatment” and “treating” as used herein refer to the management and care of a patient for the purpose of combating a condition, disease or disorder. The term is intended to include the full spectrum of treatments for a given condition from which the patient is suffering, such as administration of the active compound for the purpose of: alleviating or relieving symptoms or complications; delaying the progression of the condition, disease or disorder; curing or eliminating the condition, disease or disorder; and/or preventing the condition, disease or disorder, wherein “preventing” or “prevention” is to be understood to refer to the management and care of a patient for the purpose of hindering the development of the condition, disease or disorder, and includes the administration of the active compounds to prevent the onset of symptoms or complications. The patient to be treated is preferably a mammal, in particular a human being. Treatment of animals, such as dogs, cats, cows, sheep and pigs, is, however, also within the scope of the present invention.

### DESCRIPTION OF THE INVENTION

In one embodiment of the invention, R¹ represents tert-butyl.

In one embodiment of the invention, R¹ represents hydrogen, halogen (e.g. F or Cl) or halogen-substituted phenyl (e.g. F—C₆H₄—).

In one embodiment of the invention, R¹² represents C₁₋₆-fluoroalkyl.

In a further embodiment of the invention, R¹² represents trifluoromethyl.

In another embodiment of the invention, R¹ and R² together with the benzene ring, or, with the proviso that m is 0, R² and R³ together with the benzene ring or R³ and R⁴ together with the benzene ring form a 9-11-membered bicyclic ring system which may be fully conjugated or partly saturated, and which may optionally be substituted with one or more substituents selected among halogen, hydroxy, nitro, cyano, C₁₋₆alkyl, C₁₋₆-alkenyl, C₁₋₆alkynyl, C₃₋₈cycloalkyl, C₄₋₈cycloalkenyl, C₁₋₆alkoxy, C₁₋₆haloalkoxy and C₁₋₆-haloalkyl.

In a further embodiment of the invention, R¹ and R² together with the benzene ring form a naphthalene ring system.

In a still further embodiment of the invention, R² represents halogen (e.g. Cl).

In yet another embodiment of the invention, m is 0, and R³ and R⁴ together with the benzene ring form a tetrahydronaphthalene ring system.

In another embodiment of the invention, m is 0, and R³ and R⁴ together with the benzene ring form an indane ring system.

In a further embodiment of the invention, m is 0, and R³ represents —SR¹⁷, —S(O)R¹⁷ or —S(O)₂R¹⁷; in a further aspect of the latter embodiment of the invention, R¹⁷ represents arylC₁₋₆alkyl (e.g. benzyl).

In yet another embodiment of the invention, m is 0, and R³ represents —SCH₃, —S(O)CH₃ or —S(O)₂CH₃.

In a still further embodiment of the invention, m is 0, and R³ represents —OR¹⁷.

In another embodiment of the invention, m is 0, and R³ represents —NH₂, —NH—COR¹⁷ or —NH—S(O)₂R¹⁷.

In another embodiment of the invention, m is 0, and R³ represents chlorine or fluorine.

In a further embodiment of the invention, m is 0, and R³ represents —S(O)₂NR¹⁷R¹⁸; in a further aspect of the latter embodiment of the invention, NR¹⁷R¹⁸ represents morpholin-4-yl.

In another embodiment of the invention, m is 0, and R³ represents optionally substituted aryl or heteroaryl.

In a further embodiment of the invention, m is 0, and R³ represents optionally substituted aryl.

In a still further embodiment of the invention, m is 0, and R³ represents optionally substituted phenyl. In one aspect of the latter embodiment, R³ is phenyl substituted with one or more substituents selected among halogen, cyano, C₁₋₆alkyl, C₁₋₆haloalkyl, C₁₋₆haloalkoxy, C₁₋₆alkoxy, —S(O)ₚR¹⁷ and —C(O)—R¹⁷, wherein p and R¹⁷ are as defined above.

In another embodiment of the invention, R⁴ represents halogen (e.g. Cl) or hydroxy.

In still another embodiment of the invention, R¹ represents tert-butyl, and R⁴ represents C₁₋₆alkyl.

In yet another embodiment of the invention, one substituent among R⁵, R⁶ and R⁷ represents —S(O)₂CF₃, and one of the remaining substituents among R⁵, R⁶ and R⁷ represents chloro.

In a further embodiment of the invention, one substituent among R⁵, R⁶ and R⁷ represents 4-trifluoromethylsulfonyl, and one of the remaining substituents among R⁵, R⁶ and R⁷ represents 2-chloro. In a further aspect of this latter embodiment, the single remaining substituent among R⁵, R⁶ and R⁷ represents hydrogen.

Specific, individual embodiments of compounds according to the present invention include each of the following compounds:
5-tert-Butyl-3′,5′-difluoro-4-hydroxy-2-methyl-biphenyl-3-carboxylic acid (2-chloro-4-trifluoromethanesulfonyl-phenyl)-amide;
5-tert-Butyl-4-hydroxy-2-methyl-4′-trifluoromethoxy-biphenyl-3-carboxylic acid (2-chloro-4-trifluoromethanesulfonyl-phenyl)-amide;
5-tert-Butyl-4-hydroxy-2-methyl-3′-trifluoromethyl-biphenyl-3-carboxylic acid (2-chloro-4-trifluoromethanesulfonyl-phenyl)-amide;
3-Bromo-5-tert-butyl-N-(2-chloro-4-trifluoromethanesulfonyl-phenyl)-6-hydroxy-2-methyl-benzamide;
5-tert-Butyl-4′-fluoro-4-hydroxy-2-methyl-biphenyl-3-carboxylic acid (2-chloro-4-trifluoromethanesulfonyl-phenyl)-amide;
5-tert-Butyl-4-hydroxy-2-methyl-4′-methylsulfanyl-biphenyl-3-carboxylic acid (2-chloro-4-trifluoromethanesulfonyl-phenyl)-amide;
5-tert-Butyl-3′,4′-difluoro-4-hydroxy-2-methyl-biphenyl-3-carboxylic acid (2-chloro-4-trifluoromethanesulfonyl-phenyl)-amide;
5-tert-Butyl-4′-cyano-4-hydroxy-2-methyl-biphenyl-3-carboxylic acid (2-chloro-4-trifluoromethanesulfonyl-phenyl)-amide;
3-Bromo-5-tert-butyl-N-(3-chloro-4-trifluoromethylsulfanyl-phenyl)-6-hydroxy-2-methyl-benzamide;
5-tert-Butyl-3′,4′-difluoro-4-hydroxy-2-methyl-biphenyl-3-carboxylic acid (3-chloro-4-trifluoromethylsulfanyl-phenyl)-amide;
5-tert-Butyl-3′-chloro-4′-fluoro-4-hydroxy-2-methyl-biphenyl-3-carboxylic acid (2-chloro-4-trifluoromethanesulfonyl-phenyl)-amide;
5-tert-Butyl-4′-fluoro-4-hydroxy-2,3′-dimethyl-biphenyl-3-carboxylic acid (2-chloro-4-trifluoromethanesulfonyl-phenyl)-amide;
3-Chloro-5-tert-butyl-N-(2-chloro-4-trifluoromethanesulfonyl-phenyl)-6-hydroxy-2-methyl-benzamide;
5-tert-Butyl-4-hydroxy-4′-methanesulfonyl-2-methyl-biphenyl-3-carboxylic acid (2-chloro-4-trifluoromethanesulfonyl-phenyl)-amide;
5-tert-Butyl-3′,4′-difluoro-4-hydroxy-2-methyl-biphenyl-3-carboxylic acid (3-chloro-4-trifluoromethanesulfinyl-phenyl)-amide;
1-Hydroxy-naphthalene-2-carboxylic acid (3-trifluoromethanesulfonyl-phenyl)-amide;
1-Hydroxy-naphthalene-2-carboxylic acid (3-trifluoromethylsulfanyl-phenyl)-amide;
2-Hydroxy-naphthalene-1-carboxylic acid (3-trifluoromethanesulfonyl-phenyl)-amide;
3-Hydroxy-naphthalene-2-carboxylic acid (3-trifluoromethanesulfonyl-phenyl)-amide;
1-Hydroxy-5,6,7,8-tetrahydro-naphthalene-2-carboxylic acid (2-chloro-4-trifluoromethanesulfonyl-phenyl)-amide;
1-Hydroxy-5,6,7,8-tetrahydro-naphthalene-2-carboxylic acid (4-trifluoromethylsulfanyl-phenyl)-amide;
4-Benzenesulfonyl-1-hydroxy-naphthalene-2-carboxylic acid (4-trifluoromethylsulfanyl-phenyl)-amide;
4-Chloro-1-hydroxy-naphthalene-2-carboxylic acid (4-trifluoromethylsulfanyl-phenyl)-amide;
3-Adamantan-1-yl-N-(2-chloro-4-trifluoromethanesulfonyl-phenyl)-2-hydroxy-5-methyl-benzamide;
4-Chloro-1-hydroxy-naphthalene-2-carboxylic acid (2-chloro-4-trifluoromethanesulfonyl-phenyl)-amide;
3-tert-Butyl-N-(2-chloro-4-trifluoromethanesulfonyl-phenyl)-2-hydroxy-5-methyl-benzamide;
4-Chloro-1-hydroxy-naphthalene-2-carboxylic acid (3-chloro-4-trifluoromethylsulfanyl-phenyl)-amide;
3-Adamantan-1-yl-N-(3-chloro-4-trifluoromethylsulfanyl-phenyl)-2-hydroxy-5-methyl-benzamide;
3-Adamantan-1-yl-N-(3-chloro-4-trifluoromethylsulfanyl-phenyl)-2-hydroxy-5-methyl-benzamide;
6-Adamantan-1-yl-5-hydroxy-indan-4-carboxylic acid (2-chloro-4-trifluoromethanesulfonyl-phenyl)-amide;
4-(4-Fluoro-phenylsulfanyl)-1-hydroxy-naphthalene-2-carboxylic acid (2-chloro-4-trifluoromethanesulfonyl-phenyl)-amide;
5-Adamantan-1-yl-2′,4′-difluoro-4-hydroxy-biphenyl-3-carboxylic acid (2-chloro-4-trifluoromethanesulfonyl-phenyl)-amide;
4-(4-Fluoro-benzenesulfinyl)-1-hydroxy-naphthalene-2-carboxylic acid (2-chloro-4-trifluoromethanesulfonyl-phenyl)-amide;
5-tert-Butyl-2′,4′-difluoro-4-hydroxy-biphenyl-3-carboxylic acid (2-chloro-4-trifluoromethanesulfonyl-phenyl)-amide;
2′,4′-Difluoro-4-hydroxy-5-(1-methyl-cyclohexyl)-biphenyl-3-carboxylic acid (2-chloro-4-trifluoromethanesulfonyl-phenyl)-amide;
4-(4-Ethanesulfonyl-phenyl)-1-hydroxy-naphthalene-2-carboxylic acid (2-chloro-4-trifluoromethanesulfonyl-phenyl)-amide;
1-Hydroxy-4-(4-methoxy-phenyl)-naphthalene-2-carboxylic acid (2-chloro-4-trifluoromethanesulfonyl-phenyl)-amide;
4-(4-Fluoro-phenyl)-1-hydroxy-naphthalene-2-carboxylic acid (2-chloro-4-trifluoromethanesulfonyl-phenyl)-amide;
1-Hydroxy-4-p-tolyl-naphthalene-2-carboxylic acid (2-chloro-4-trifluoromethanesulfonyl-phenyl)-amide;
4-(4-Cyano-phenyl)-1-hydroxy-naphthalene-2-carboxylic acid (2-chloro-4-trifluoromethanesulfonyl-phenyl)-amide;
4-(3,5-Difluoro-phenyl)-1-hydroxy-naphthalene-2-carboxylic acid (2-chloro-4-trifluoromethanesulfonyl-phenyl)-amide;
1-Hydroxy-4-(3-trifluoromethyl-phenyl)-naphthalene-2-carboxylic acid (2-chloro-4-trifluoromethanesulfonyl-phenyl)-amide;
4-(4-Acetyl-phenyl)-1-hydroxy-naphthalene-2-carboxylic acid (2-chloro-4-trifluoromethanesulfonyl-phenyl)-amide;
2-Hydroxy-5-methyl-3-(1,1,2,2-tetramethyl-propyl)-N-(4-trifluoromethanesulfonyl-phenyl)-benzamide;
4′-Bromo-5-tert-butyl-4-hydroxy-biphenyl-3-carboxylic acid (2-chloro-4-trifluoromethanesulfonyl-phenyl)-amide;
5-tert-Butyl-2′,4′-difluoro-4-hydroxy-biphenyl-3-carboxylic acid (4-trifluoromethanesulfonyl-phenyl)-amide;
2′,4′-Difluoro-4-hydroxy-5-(1-methyl-cyclopentyl)-biphenyl-3-carboxylic acid (4-trifluoromethanesulfonyl-phenyl)-amide;
5-(1,1-Dimethyl-propyl)-2′,4′-difluoro-4-hydroxy-biphenyl-3-carboxylic acid (2-chloro-4-trifluoromethanesulfonyl-phenyl)-amide;
5-(1-Cyclopropyl-1-methyl-ethyl)-2′,4′-difluoro-4-hydroxy-biphenyl-3-carboxylic acid (2-chloro-4-trifluoromethanesulfonyl-phenyl)-amide;
5-Chloro-N-(2-chloro-4-trifluoromethanesulfonyl-phenyl)-3-(1,1-dimethyl-propyl)-2-hydroxy-benzamide;
N-(2-Chloro-4-trifluoromethanesulfonyl-phenyl)-2-hydroxy-5-methanesulfonyl-3-(1,1,2,2-tetramethyl-propyl)-benzamide;
3-tert-Butyl-N-(2-chloro-4-trifluoromethanesulfonyl-phenyl)-2-hydroxy-5-methanesulfonyl-6-methyl-benzamide;
3-tert-Butyl-N-(2-chloro-4-trifluoromethanesulfonyl-phenyl)-6-ethyl-2-hydroxy-benzamide;
3,5-Di-tert-butyl-N-(2-chloro-4-trifluoromethanesulfonyl-phenyl)-2-ethyl-6-hydroxy-benzamide;
6-tert-Butyl-5-hydroxy-indan-4-carboxylic acid (2-chloro-4-trifluoromethanesulfonyl-phenyl)-amide;
3-tert-Butyl-N-(2-chloro-4-trifluoromethanesulfonyl-phenyl)-2-hydroxy-6-methyl-5-methylsulfanyl-benzamide;
3-tert-Butyl-2-hydroxy-5,6,7,8-tetrahydro-naphthalene-1-carboxylic acid (2-chloro-4-trifluoromethanesulfonyl-phenyl)-amide;
3-tert-Butyl-N-(2-chloro-4-trifluoromethanesulfonyl-phenyl)-2-hydroxy-5-methanesulfinyl-6-methyl-benzamide;
3-tert-Butyl-N-(2-chloro-4-trifluoromethanesulfonyl-phenyl)-2-hydroxy-5-methoxy-benzamide
3-tert-Butyl-N-(2-chloro-4-trifluoromethanesulfonyl-phenyl)-2-hydroxy-6-isopropyl-benzamide
5-tert-Butyl-3′,5′-difluoro-4-hydroxy-2-methyl-biphenyl-3-carboxylic acid (4-trifluoromethanesulfonyl-phenyl)-amide;
5-tert-Butyl-4′-fluoro-4-hydroxy-2-methyl-biphenyl-3-carboxylic acid (4-trifluoromethanesulfonyl-phenyl)-amide;
5-tert-Butyl-3′,5′-difluoro-4-hydroxy-2-methyl-biphenyl-3-carboxylic acid (3-chloro-4-trifluoromethanesulfonyl-phenyl)-amide;
5-tert-Butyl-4′-fluoro-4-hydroxy-2-methyl-biphenyl-3-carboxylic acid (3-chloro-4-trifluoromethanesulfonyl-phenyl)-amide;
3-tert-Butyl-N-(2-chloro-4-trifluoromethanesulfonyl-phenyl)-5-fluoro-2-hydroxy-6-methyl-benzamide;
3-tert-Butyl-5-chloro-N-(3-chloro-4-trifluoromethanesulfonyl-phenyl)-2-hydroxy-6-methyl-benzamide;
3-tert-Butyl-N-(2-chloro-4-trifluoromethanesulfonyl-phenyl)-2-hydroxy-5-iodo-6-methyl-benzamide;
3-tert-Butyl-5-chloro-2-hydroxy-6-methyl-N-(4-trifluoromethanesulfonyl-phenyl)-benzamide;
5-tert-Butyl-3′,4′-difluoro-4-hydroxy-2-methyl-biphenyl-3-carboxylic acid (4-trifluoromethanesulfonyl-phenyl)-amide;
5-tert-Butyl-3′,4′-difluoro-4-hydroxy-2-methyl-biphenyl-3-carboxylic acid □(3-chloro-4-trifluoromethanesulfonyl-phenyl)-amide;
5-tert-Butyl-4-hydroxy-4′-methoxy-2-methyl-biphenyl-3-carboxylic acid (2-chloro-4-trifluoromethanesulfonyl-phenyl)-amide;
3-tert-Butyl-N-(2-chloro-4-trifluoromethanesulfonyl-phenyl)-5-(5-cyano-thiophen-2-yl)-2-hydroxy-6-methyl-benzamide;
5-tert-Butyl-4′-dimethylsulfamoyl-4-hydroxy-2-methyl-biphenyl-3-carboxylic acid (2-chloro-4-trifluoromethanesulfonyl-phenyl)-amide;
5-tert-Butyl-4-hydroxy-2-methyl-4′-(morpholine-4-sulfonyl)-biphenyl-3-carboxylic acid (2-chloro-4-trifluoromethanesulfonyl-phenyl)-amide;
3-1,3-Benzodioxol-5-yl-5-tert-butyl-N-(2-chloro-4-trifluoromethanesulfonyl-phenyl)-6-hydroxy-2-methyl-benzamide;
5-tert-Butyl-4-hydroxy-3′-methoxy-2-methyl-biphenyl-3-carboxylic acid (2-chloro-4-trifluoromethanesulfonyl-phenyl)-amide;
4-(3,4-Difluorophenyl)-1-hydroxynaphthalene-2-carboxylic acid (2-chloro-4-trifluoromethanesulfonylphenyl)amide;
1-Hydroxy-4-(4-trifluoromethylphenyl)naphthalene-2-carboxylic acid (2-chloro-4-trifluoromethanesulfonylphenyl)amide;
1-Hydroxy-4-(3-methoxyphenyl)naphthalene-2-carboxylic acid (2-chloro-4-trifluoromethanesulfonylphenyl)amide;
4-Bromo-1-hydroxynaphthalene-2-carboxylic acid (2-chloro-4-trifluoromethanesulfonylphenyl)amide;
5-Bromo-3-tert-butyl-N-(2-chloro-4-trifluoromethanesulfonylphenyl)-2-hydroxybenzamide;
5-tert-Butyl-3′,4′-difluoro-4-hydroxybiphenyl-3-carboxylic acid (2-chloro-4-trifluoromethanesulfonylphenyl)amide;
5-tert-Butyl-4′-fluoro-4-hydroxybiphenyl-3-carboxylic acid (2-chloro-4-trifluoromethanesulfonylphenyl)amide;
5-tert-Butyl-3′,5′-difluoro-4-hydroxybiphenyl-3-carboxylic acid (2-chloro-4-trifluoromethanesulfonylphenyl)amide;
3,5-Di-tert-butyl-N-(2-chloro-4-trifluoromethanesulfonylphenyl)-2-hydroxybenzamide;
3-tert-Butyl-N-(2-chloro-4-trifluoromethanesulfonylphenyl)-2-hydroxy-6-methylbenzamide;
1-Hydroxy-3-methylnaphthalene-2-carboxylic acid (2-chloro-4-trifluoromethane-sulfonylphenyl)amide;
4-Bromo-1-hydroxy-3-methylnaphthalene-2-carboxylic acid (2-chloro-4-trifluoromethanesulfonylphenyl)amide;
3-Bromo-5-tert-butyl-6-hydroxy-2-methyl-N-(2-nitro-4-trifluoromethanesulfonyl-phenyl)-benzamide;
5-tert-Butyl-3′-fluoro-4-hydroxy-2-methyl-biphenyl-3-carboxylic acid (2-chloro-4-trifluoromethanesulfonyl-phenyl)-amide;
5′-tert-Butyl-3′-(2-chloro-4-trifluoromethanesulfonyl-phenylcarbamoyl)-4′-hydroxy-2′-methyl-biphenyl-3-carboxylic acid methyl ester;
3-tert-Butyl-N-(3-chloro-4-trifluoromethanesulfonyl-phenyl)-5-fluoro-2-hydroxy-6-methyl-benzamide;
3-tert-Butyl-5-chloro-N-(2-dimethylamino-4-trifluoromethanesulfonyl-phenyl)-2-hydroxy-6-methyl-benzamide;
3-tert-Butyl-5-chloro-N-(2-dipropylamino-4-trifluoromethanesulfonyl-phenyl)-2-hydroxy-6-methyl-benzamide;
3-tert-Butyl-(2-chloro-4-trifluoromethanesulfonyl-phenyl)-2-hydroxy-6-methyl-5-(morpholine-4-sulfonyl)-benzamide;
3-tert-Butyl-N-(2-chloro-4-trifluoromethanesulfonylphenyl)-5-dimethylsulfamoyl-2-hydroxy-6-methyl-benzamide;
3-tert-Butyl-N-(2-chloro-4-trifluoromethanesulfonylphenyl)-5-dimethylsulfamoyl-2-hydroxy-6-methyl-benzamide;
3-tert-Butyl-N-(2-chloro-4-trifluoromethanesulfonyl-phenyl)-6-ethyl-2-hydroxy-5-methylsulfanyl-benzamide;
3-tert-Butyl-N-(2-chloro-4-trifluoromethanesulfonyl-phenyl)-6-ethyl-2-hydroxy-5-methanesulfinyl-benzamide;
3-tert-Butyl-N-(2-chloro-4-trifluoromethanesulfonyl-phenyl)-6-ethyl-2-hydroxy-5-methanesulfonyl-benzamide;
3-tert-Butyl-N-(2-chloro-4-trifluoromethanesulfonyl-phenyl)-2-hydroxy-6-isopropyl-5-methylsulfanyl-benzamide;
3-tert-Butyl-N-(2-chloro-4-trifluoromethanesulfonyl-phenyl)-2-hydroxy-6-isopropyl-5-methanesulfinyl-benzamide;
3-tert-Butyl-N-(2-chloro-4-trifluoromethanesulfonyl-phenyl)-2-hydroxy-6-isopropyl-5-methanesulfonyl-benzamide;
3-tert-Butyl-N-(2-chloro-4-trifluoromethanesulfonyl-phenyl)-2-hydroxy-6-methyl-5-(2,2,2-trifluoro-ethylsulfanyl)-benzamide;
3-tert-Butyl-N-(2-chloro-4-trifluoromethanesulfonyl-phenyl)-2-hydroxy-5-trifluoromethylsulfanyl-benzamide;
3-tert-Butyl-N-(2-chloro-4-trifluoromethanesulfonyl-phenyl)-2-hydroxy-5-isopropylsulfanyl-6-methyl-benzamide;
5-tert-Butoxy-3-tert-butyl-N-(2-chloro-4-trifluoromethanesulfonyl-phenyl)-2-hydroxy-benzamide;
3-tert-Butyl-N-(2-chloro-4-trifluoromethanesulfonyl-phenyl)-6-ethyl-2-hydroxy-5-methoxy-benzamide;
N-(2-Chloro-4-trifluoromethanesulfonyl-phenyl)-2-hydroxy-3-isopropyl-6-methyl-5-methylsulfanyl-benzamide;
N-(2-Chloro-4-trifluoromethanesulfonyl-phenyl)-2-hydroxy-3-isopropyl-5-methanesulfinyl-6-methyl-benzamide;
N-(2-Chloro-4-trifluoromethanesulfonyl-phenyl)-2-hydroxy-3-isopropyl-6-methyl-5-methylsulfonyl-benzamide;
3-tert-Butyl-N-(2-methoxy-4-trifluoromethanesulfonyl-phenyl)-6-methyl-2-hydroxy-5-methylsulfanyl-benzamide;
3-tert-Butyl-N-(2-methoxy-4-trifluoromethanesulfonyl-phenyl)-6-methyl-2-hydroxy-5-methanesulfinyl-benzamide;
3-tert-Butyl-N-(2-cyano-4-trifluoromethanesulfonyl-phenyl)-6-methyl-2-hydroxy-5-methanesulfonyl-benzamide;
3-tert-Butyl-N-(2-cyano-4-trifluoromethanesulfonyl-phenyl)-6-methyl-2-hydroxy-5-fluoro-benzamide;
3-tert-Butyl-N-(2-methyl-4-trifluoromethanesulfonyl-phenyl)-6-methyl-2-hydroxy-5-fluoro-benzamide;
3-tert-Butyl-N-(2-methoxy-4-trifluoromethanesulfonyl-phenyl)-6-methyl-2-hydroxy-5-fluoro-benzamide;
3-tert-Butyl-N-(2-cyano-4-trifluoromethanesulfonyl-phenyl)-6-methyl-2-hydroxy-5-chloro-benzamide;
3-tert-Butyl-N-(2-methyl-4-trifluoromethanesulfonyl-phenyl)-6-methyl-2-hydroxy-5-chloro-benzamide;
3-tert-Butyl-N-(2-methoxy-4-trifluoromethanesulfonyl-phenyl)-6-methyl-2-hydroxy-5-chloro-benzamide;
3-tert-Butyl-N-(2-cyano-4-trifluoromethanesulfonyl-phenyl)-2-hydroxy-6-methyl-5-methylsulfanyl-benzamide;
3-tert-Butyl-2-hydroxy-6-methyl-5-methylsulfanyl-N-(2-methyl-4-trifluoromethanesulfonyl-phenyl)-benzamide;
N-(2-Bromo-4-trifluoromethanesulfonyl-phenyl)-3-tert-butyl-2-hydroxy-6-methyl-5-methylsulfanyl-benzamide;
3-tert-Butyl-N-(2-cyano-4-trifluoromethanesulfonyl-phenyl)-2-hydroxy-6-methyl-5-methylsulfinyl-benzamide;
3-tert-Butyl-2-hydroxy-6-methyl-5-methylsulfinyl-N-(2-methyl-4-trifluoromethanesulfonyl-phenyl)-benzamide;
N-(2-Bromo-4-trifluoromethanesulfonyl-phenyl)-3-tert-butyl-2-hydroxy-6-methyl-5-methylsulfinyl-benzamide;
3-tert-Butyl-N-(2-cyano-4-trifluoromethanesulfonyl-phenyl)-2-hydroxy-5-methoxy-6-methyl-benzamide;
3-tert-Butyl-2-hydroxy-5-methoxy-6-methyl-N-(2-methyl-4-trifluoromethanesulfonyl-phenyl)-benzamide;
N-(2-Bromo-4-trifluoromethanesulfonyl-phenyl)-3-tert-butyl-2-hydroxy-5-methoxy-6-methyl-benzamide;
N-(2-Chloro-4-trifluoromethanesulfonyl-phenyl)-3-tert-butyl-2-hydroxy-5-methoxy-6-methyl-benzamide;
1-Hydroxy-3-methyl-4-methylsulfanyl-naphthalene-2-carboxylic acid (2-chloro-4-trifluoromethanesulfonyl-phenyl)-amide;
1-Hydroxy-4-methanesulfinyl-3-methyl-naphthalene-2-carboxylic acid (2-chloro-4-trifluoromethanesulfonyl-phenyl)-amide;
1-Hydroxy-4-methanesulfonyl-3-methyl-naphthalene-2-carboxylic acid (2-chloro-4-trifluoromethanesulfonyl-phenyl)-amide; and
4-Chloro-1-hydroxy-3-methyl-naphthalene-2-carboxylic acid (2-chloro-4-trifluoromethanesulfonyl-phenyl)-amide.

Further individual embodiments of compounds according to the present invention include each of the following compounds:
3-tert-Butyl-N-(2-chloro-4-trifluoromethanesulfonyl-phenyl)-2-hydroxy-6-methoxy-benzamide;
6-tert-Butyl-5-hydroxy-2,3-dihydro-benzofuran-4-carboxylic acid (2-chloro-4-trifluoromethane-sulfonyl-phenyl)-amide;
3-tert-Butyl-N-(2-chloro-4-trifluoromethanesulfonyl-phenyl)-2,5-dihydroxy-6-methyl-benzamide;
3-tert-Butyl-N-(2-chloro-4-trifluoromethanesulfonyl-phenyl)-2,5-dihydroxy-6-methoxy-benzamide;
3-tert-Butyl-N-(2-chloro-4-trifluoromethanesulfonyl-phenyl)-2,6-dihydroxy-5-methoxy-benzamide;
3,5-Di-tert-butyl-N-(2-chloro-4-trifluoromethanesulfonyl-phenyl)-2,6-dihydroxy-benzamide;
3-tert-Butyl-N-(2-chloro-4-trifluoromethanesulfonyl-phenyl)-5-cyano-2-hydroxy-6-methyl-benzamide;
3-tert-Butyl-N-(2-chloro-4-trifluoromethanesulfonyl-phenyl)-2-hydroxy-6-methyl-5-(4-methyl-piperazine-1-sulfonyl)-benzamide;
N-(2-Bromo-4-trifluoromethanesulfonyl-phenyl)-3-tert-butyl-5-fluoro-2-hydroxy-6-methyl-benzamide;
3-Acetyl-5-tert-butyl-N-(2-chloro-4-trifluoromethanesulfonyl-phenyl)-6-hydroxy-2-methyl-benzamide;
3-Acetyl-5-tert-butyl-6-hydroxy-N-(2-methoxy-4-trifluoromethanesulfonyl-phenyl)-2-methyl-benzamide;
3-tert-Butyl-N-(2-chloro-4-trifluoromethanesulfonyl-phenyl)-2-hydroxy-6-methyl-5-nitro-benzamide;
3-tert-Butyl-N-(2-chloro-4-trifluoromethanesulfonyl-phenyl)-2-hydroxy-6-methyl-5-(toluene-4-sulfonylamino)-benzamide;
3-Amino-5-tert-butyl-N-(2-chloro-4-trifluoromethanesulfonyl-phenyl)-6-hydroxy-2-methyl-benzamide;
3-Acetylamino-5-tert-butyl-N-(2-chloro-4-trifluoromethanesulfonyl-phenyl)-6-hydroxy-2-methyl-benzamide;
1-Hydroxy-4-(4-methoxyphenyl)naphthalene-2-carboxylic acid (2-methyl-4-trifluoromethanesulfonylphenyl)amide;
4-(4-Fluorophenyl)-1-hydroxynaphthalene-2-carboxylic acid (4-trifluoromethanesulfonylphenyl)amide;
4-(4-Fluorophenyl)-1-hydroxynaphthalene-2-carboxylic acid (2-methyl-4-trifluoromethanesulfonylphenyl)amide;
1-Hydroxynaphthalene-2-carboxylic acid (2-chloro-4-trifluoromethanesulfonylphenyl)amide;
1-Hydroxy-8-methylnaphthalene-2-carboxylic acid (2-chloro-4-trifluoromethanesulfonyl-phenyl)amide;
N-(3-Chloro-5-trifluoromethanesulfonyl-phenyl)-2-hydroxy-benzamide;
3-Benzylsulfanyl-5-tert-butyl-N-(2-chloro-4-trifluoromethanesulfonyl-phenyl)-6-hydroxy-2-methyl-benzamide;
3-tert-Butyl-N-(2-chloro-4-trifluoromethanesulfonyl-phenyl)-2-hydroxy-6-methyl-5-propylsulfanyl-benzamide;
3-tert-Butyl-N-(2-chloro-4-trifluoromethanesulfonyl-phenyl)-2-hydroxy-6-methyl-5-(propane-1-sulfinyl)-benzamide;
3-tert-Butyl-N-(2-chloro-4-trifluoromethanesulfonyl-phenyl)-2-hydroxy-6-methyl-5-phenylmethanesulfinyl-benzamide;
3-tert-Butyl-5-chloro-2-hydroxy-6-methyl-N-(2-propyl-4-trifluoromethanesulfonyl-phenyl)-benzamide;
3-tert-Butyl-N-(2-chloro-4-trifluoromethanesulfonyl-phenyl)-2-hydroxy-5-isobutylsulfanyl-6-methyl-benzamide;
3-tert-Butyl-5-chloro-N-(2-ethyl-4-trifluoromethanesulfonyl-phenyl)-2-hydroxy-6-methyl-benzamide;
3-tert-Butyl-N-(2-chloro-4-trifluoromethanesulfonyl-phenyl)-2-hydroxy-6-methyl-5-phenylmethanesulfonyl-benzamide;
3-tert-Butyl-N-(2-chloro-4-trifluoromethanesulfonyl-phenyl)-2-hydroxy-6-methyl-5-(propane-1-sulfonyl)-benzamide;
5-tert-Butyl-4′-cyano-4-hydroxy-2-methyl-biphenyl-3-carboxylic acid (2-methyl-4-trifluoromethanesulfonyl-phenyl)-amide;
5-tert-Butyl-4′-cyano-4-hydroxy-2-methyl-biphenyl-3-carboxylic acid (2-ethyl-4-trifluoromethanesulfonyl-phenyl)-amide;
5-tert-Butyl-4′-cyano-4-hydroxy-2-methyl-biphenyl-3-carboxylic acid (2-cyano-4-trifluoromethanesulfonyl-phenyl)-amide;
3-tert-Butyl-N-(2-chloro-4-trifluoromethanesulfonyl-phenyl)-2-hydroxy-6-methyl-5-(propane-2-sulfonyl)-benzamide;
3-tert-Butyl-N-(2-chloro-4-trifluoromethanesulfonyl-phenyl)-2-hydroxy-6-methyl-5-(propane-2-sulfinyl)-benzamide;
5-tert-Butyl-4′-cyano-4-hydroxy-2-methyl-biphenyl-3-carboxylic acid (2-propyl-4-trifluoromethanesulfonyl-phenyl)-amide;
3-tert-Butyl-5-chloro-N-(3-cyano-4-trifluoromethanesulfonyl-phenyl)-2-hydroxy-6-methyl-benzamide;
3-tert-Butyl-N-(2-chloro-4-trifluoromethanesulfonyl-phenyl)-2-hydroxy-6-methyl-5-(2-methyl-propane-1-sulfinyl)-benzamide;
N-(2-Chloro-4-trifluoromethanesulfonylphenyl)-2-hydroxy-3-isopropyl-6-methylbenzamide;
N-(2-Chloro-4-trifluoromethanesulfonylphenyl)-2-hydroxy-6-methylbenzamide;
5-Chloro-N-(2-chloro-4-trifluoromethanesulfonylphenyl)-2-hydroxybenzamide;
N-(2-Chloro-4-trifluoromethanesulfonylphenyl)-2-hydroxy-6-isopropyl-3-methylbenzamide;
N-(2-Chloro-4-trifluoromethanesulfonylphenyl)-5-fluoro-2-hydroxybenzamide;
3-Chloro-N-(2-chloro-4-trifluoromethanesulfonylphenyl)-6-hydroxy-5-isopropyl-2-methylbenzamide;
3-Chloro-N-(2-chloro-4-trifluoromethanesulfonylphenyl)-6-hydroxy-2-methylbenzamide;
3,5-Dichloro-N-(2-chloro-4-trifluoromethanesulfonylphenyl)-2-hydroxy-6-methylbenzamide;
3-Chloro-N-(2-chloro-4-trifluoromethanesulfonylphenyl)-6-hydroxy-2-isopropyl-5-methylbenzamide;
N-(2-Chloro-4-trifluoromethanesulfonylphenyl)-2-hydroxy-3,4,6-trimethylbenzamide;
4-Chloro-N-(2-chloro-4-trifluoromethanesulfonylphenyl)-2-hydroxybenzamide;
2-Hydroxybiphenyl-3-carboxylic acid (2-chloro-4-trifluoromethanesulfonylphenyl)amide;
N-(2-Chloro-4-trifluoromethanesulfonylphenyl)-2,3,4,5-tetrafluoro-6-hydroxybenzamide;
5-tert-Butyl-4-hydroxy-2-methylbiphenyl-3-carboxylic acid (2-chloro-4-trifluoromethanesulfonylphenyl)amide;
N-(2-Chloro-4-trifluoromethanesulfonylphenyl)-3,5-difluoro-2-hydroxybenzamide;
3′,5′-Difluoro-4-hydroxy-2-methylbiphenyl-3-carboxylic acid (2-chloro-4-trifluoromethanesulfonylphenyl)amide;
4′-Cyano-4-hydroxy-2-methylbiphenyl-3-carboxylic acid (2-chloro-4-trifluoromethanesulfonylphenyl)amide;
N-(2-Chloro-4-trifluoromethanesulfonylphenyl)-4-fluoro-2-hydroxybenzamide;
3′,5′-Difluoro-4-hydroxy-5-isopropyl-2-methylbiphenyl-3-carboxylic acid (2-chloro-4-trifluoromethanesulfonylphenyl)amide;
3-tert-Butyl-6-chloro-N-(2-chloro-4-trifluoromethanesulfonylphenyl)-2-hydroxybenzamide;
3-tert-Butyl-6-chloro-2-hydroxy-N-(2-methyl-4-trifluoromethanesulfonylphenyl)benzamide;
5-Chloro-2-hydroxy-4-methylbiphenyl-3-carboxylic acid (2-chloro-4-trifluoromethanesulfonylphenyl)amide;
5-Chloro-4′-fluoro-2-hydroxy-4-methylbiphenyl-3-carboxylic acid (2-chloro-4-trifluoromethanesulfonylphenyl)amide;
N-(2-Chloro-4-trifluoromethanesulfonyl-phenyl)-6-hydroxy-2,4-dimethyl-3-methylsulfanyl-benzamide;
N-(2-Chloro-4-trifluoromethanesulfonyl-phenyl)-6-hydroxy-3-methanesulfinyl-2,4-dimethyl-benzamide;
3-tert-Butyl-5-tert-butylsulfanyl-N-(2-chloro-4-trifluoromethanesulfonyl-phenyl)-2-hydroxy-6-methyl-benzamide;
3-tert-Butyl-5-tert-butylsulfinyl-N-(2-chloro-4-trifluoromethanesulfonyl-phenyl)-2-hydroxy-6-methyl-benzamide;
3-tert-Butyl-5-tert-butylsulfonyl-N-(2-chloro-4-trifluoromethanesulfonyl-phenyl)-2-hydroxy-6-methyl-benzamide;
N-(2-Chloro-4-trifluoromethanesulfonyl-phenyl)-2,3-difluoro-6-hydroxy-5-methyl-benzamide;
4-(4-Dimethylamino-phenyl)-1-hydroxy-naphthalene-2-carboxylic acid (2-chloro-4-trifluoromethanesulfonyl-phenyl)-amide;
2,5-Di-tert-butyl-N-(2-chloro-4-trifluoromethanesulfonyl-phenyl)-6-hydroxy-3-methoxy-benzamide;
6-tert-Butyl-2-[(2-chloro-4-trifluoromethanesulfonyl-phenylamino)-hydroxy-methyl]-3,4-dimethoxy-phenol;
3-tert-Butyl-N-(2-chloro-4-trifluoromethanesulfonyl-phenyl)-2,6-dihydroxy-benzamide; and
3-tert-Butyl-N-(2-chloro-4-trifluoromethanesulfonyl-phenyl)-2-hydroxy-6-isopropyl-5-methoxy-benzamide.

Compounds according to formula I may comprise chiral carbon atoms, chiral sulfur atoms or carbon-carbon double bonds which may give rise to stereoisomeric forms, e.g. enantiomers, diastereomers and/or geometric isomers. The present invention relates to all such isomers, either in pure form or as mixtures thereof. Pure isomeric forms may either be prepared from intermediates which are pure isomers themselves, by purification of a mixture of isomers after the synthesis, or by a combination of the two methods. Purification of isomeric forms is well known in the art, e.g. as described by Jaques in Enantiomers, Racemates and Resolution, Wiley, 1981.

The compounds of the present invention are useful in the treatment of diseases or states that benefit from an increase in the mitochondrial respiration.

The compounds of the present invention are believed to be particular well-suited for the treatment of obesity as such or preventing weight gain and for the treatment of conditions, diseases or disorders where obesity is involved in the etiology. In one embodiment, the invention thus provides a method of treating the metabolic syndrome, insulin resistance, dyslipidemia, hypertension, obesity, type 2 diabetes, type 1 diabetes, diabetic late complications including cardiovascular diseases, cardiovascular disorders, disorders of lipid metabolism, neurodegenerative and psychiatric disorders, dysregulation of intraocular pressure including glaucoma, atherosclerosis, hypertension, coronary heart disease, gallbladder disease, osteoarthritis or cancer.

More specifically such conditions include the metabolic syndrome, type 2 diabetes (especially in obese patients), diabetes as a consequence of obesity, insulin resistance, hyperglycemia, prandial hyperglycemia, hyperinsulinemia, impaired glucose tolerance (IGT), impaired fasting glucose (IFG), increased hepatic glucose production, type 1 diabetes, LADA, pediatric diabetes, dyslipidemia (especially in obese patients), diabetic dyslipidemia, hyperlipidemia, hypertriglyceridemia, hyperlipoproteinemia, micro-/macroalbuminuria, nephropathy, retinopathy, neuropathy, diabetic ulcers, cardiovascular diseases, arteriosclerosis, coronary artery disease, cardiac hypertrophy, myocardial ischemia, heart insufficiency, congestional heart failure, stroke, myocardial infarction, arrhythmia, decreased blood flow, erectile dysfunction (male or female), myopathy, loss of muscle tissue, muscle wasting, muscle catabolism, osteoporosis, decreased linear growth, neurodegenerative and psychiatric disorders, Alzheimers disease, neuronal death, impaired cognitive function, depression, anxiety, eating disorders, appetite regulation, migraine, epilepsia, addiction to chemical substances, disorders of intraocular pressure, bacterial infections, mycobacterial infections. In the present context cancer is intended to include forms such as hematological cancer, e.g. leukemia, acute myeloid leukemia, chronic myeloid leukemia, chronic lymphatic leukemia, myelodysplasia, multiple myeloma, Hodgkin's disease, or solid tumor forms, such as fibrosarcom, small or non-small cell long carcinoma, gastric, intestinal or colorectal cancer, prostate, endometrial, ovarian or breast cancer, brain, head or neck cancer, cancer in the urinary tract, such as kidney or bladder cancer, malignant melanoma, liver cancer, uterine and pancreatic cancer.

In another embodiment, the invention relates to the use of a chemical uncoupler compound according to the present invention for maintaining a weight loss.

The use of compounds according to the present invention in the treatment of obesity may very likely reduce or eliminate side-effects such as irritation of the skin, glaucoma, etc., that are known to occur in connection with treatment of obesity with DNP and other chemical uncouplers that have narrow safety windows.

Uncouplers may also reduce insulin release from β-cells, and may thus be useful in providing β-cell rest. Inducing β-cell rest may be useful in connection with β-cell transplantation, and it has also been described that inducing β-cell rest may be useful in preventing diabetes.

Obesity drugs which regulate the appetite and reduce food intake often suffer from lack of long-term efficiency in terms of body weight loss because the body in response to the treatment lowers the rate of the metabolism. In contrast hereto, compounds of the present invention increase the metabolism, and they are therefore believed to be particularly suited for maintaining a weight loss.

Compounds of the present invention are also believed to be particularly well suited for the treatment of conditions, diseases or disorders where reactive oxygen species are involved in the etiology, and wherein a reduction in the amount of reactive oxygen species is beneficial. In one embodiment, the invention thus provides a method of treating, and in particular preventing, ageing and damage to the heart, endothelial cells and neuronal tissue, diabetic microvascular diseases in the retina, the renal glomerus and the peripheral nerve cells, the method comprising administering to a patient in need thereof a therapeutically effective amount of one or more compound of the present invention to a patient need thereof.

The subject (patient) may be any mammal suffering from a condition benefiting from increased mitochondrial respiration. Such mammals may include, for instance, horses, cows, sheep, pigs, mice, rats, dogs, cats, primates such as chimpanzees, gorillas and rhesus monkeys, and, in particular, humans.

It is well-known that many compounds used to combat insects or parasites, i.e. insecticides or parasiticides, respectively, are chemical uncouplers. It is thus believed that chemical uncoupler compounds according to the present invention may be useful as insecticides or parasiticides.

In methods of the present invention, a compound of the present invention may be administered alone or in combination with one or more other therapeutically active compounds, either concomitantly or sequentially, and in any suitable ratios. Such other therapeutically active compounds may, for example, be selected from antidiabetic agents, antihyperlipidemic agents, antiobesity agents, antihypertensive agents and agents for the treatment of complications resulting from, or associated with, diabetes.

Suitable antidiabetic agents include insulin, GLP-1 (glucagon like peptide-1) derivatives such as those disclosed in WO 98/08871 (Novo Nordisk A/S), the contents of which are incorporated herein by reference, as well as orally active hypoglycemic agents.

Suitable orally active hypoglycemic agents include imidazolines, sulfonylureas, biguanides, meglitinides, oxadiazolidinediones, thiazolidinediones, insulin sensitizers, α-glucosidase inhibitors, agents acting on the ATP-dependent potassium channel of the pancreatic 1-cells, e.g. potassium channel openers such as those disclosed in WO 97/26265, WO 99/03861 and WO 00/37474 (Novo Nordisk A/S), the contents of all of which are incorporated herein by reference, potassium channel openers such as ormitiglinide, potassium channel blockers such as nateglinide or BTS-67582, glucagon antagonists such as those disclosed in WO 99/01423 and WO 00/39088 (Novo Nordisk A/S and Agouron Pharmaceuticals, Inc.), the contents of both of which are incorporated herein by reference, GLP-1 agonists such as those disclosed in WO 00/42026 (Novo Nordisk A/S and Agouron Pharmaceuticals, Inc.), the contents of which are incorporated herein by reference, DPP-IV (dipeptidyl peptidase-IV) inhibitors, PTPase (protein tyrosine phosphatase) inhibitors, glucokinase activators, such as those described in WO 02/08209 to Hoffmann La Roche, inhibitors of hepatic enzymes involved in stimulation of gluconeogenesis and/or glycogenolysis, glucose uptake modulators, GSK-3 (glycogen synthase kinase-3) inhibitors, compounds modifying the lipid metabolism such as antihyperlipidemic agents and antilipidemic agents, compounds lowering food intake, and PPAR (peroxisome proliferator-activated receptor) and RXR (retinoid X receptor) agonists such as ALRT-268, LG-1268 or LG-1069.

In one embodiment of a method of the invention, a compound of the present invention may be administered in combination with insulin or an insulin analogue.

In a further embodiment, a compound of the present invention may be administered in combination with a sulfonylurea, e.g. tolbutamide, chlorpropamide, tolazamide, glibenclamide, glipizide, glimepiride, glicazide or glyburide.

In another embodiment, a compound of the present invention may be administered in combination with a biguanide, e.g. metformin.

In yet another embodiment of a method of the present invention, a compound of the present invention may be administered in combination with a meglitianide, e.g. repaglinide or senaglinide/nateglinide.

In a still further embodiment, a compound of the present invention may be administered in combination with a thiazolidinedione insulin sensitizer, e.g. troglitazone, ciglitazone, pioglitazone, rosiglitazone, isaglitazone, darglitazone, englitazone, CS-011/CI-1037 or T 174, or a compound disclosed in WO 97/41097 (e.g. 5-[[4-[3-methyl-4-oxo-3,4-dihydro-2-quinazolinyl]-methoxy]phenylmethyl]thiazolidine-2,4-dione), WO 97/41119, WO 97/41120, WO 00/41121 or WO 98/45292, the contents of all of which are incorporated herein by reference.

In another embodiment, a compound of the present may be administered in combination with an insulin sensitizer such as, e.g., GI 262570, YM-440, MCC-555, JTT-501, AR-H039242, KRP-297, GW-409544, CRE-16336, AR-H049020, LY510929, MBX-102, CLX-0940, GW-501516 or a compound disclosed in WO 99/19313 (NN622/DRF-2725), WO 00/50414, WO 00/63191, WO 00/63192, WO 00/63193 and WO 00/23425, WO 00/23415, WO 00/23451, WO 00/23445, WO 00/23417, WO 00/23416, WO 00/63153, WO 00/63196, WO 00/63209, WO 00/63190 or WO 00/63189, the contents of all of which are incorporated herein by reference.

In one embodiment, a compound of the present invention may be administered in combination with an α-glucosidase inhibitor, e.g. voglibose, emiglitate, miglitol or acarbose.

In a further embodiment, a compound of the present invention may be administered in combination with a glycogen phosphorylase inhibitor, e.g. a compound as described in WO 97/09040.

In another embodiment, a compound of the present invention may be administered in combination with a glucokinase activator.

In one embodiment, a compound of the present invention may be administered in combination with an agent acting on the ATP-dependent potassium channel of the pancreatic β-cells, e.g. tolbutamide, glibenclamide, glipizide, glicazide, BTS-67582 or repaglinide.

In another embodiment, a compound of the present invention may be administered in combination with nateglinide.

In one embodiment, a compound of the present invention may be administered in combination with an antihyperlipidemic agent or an antilipidemic agent, e.g. cholestyramine, colestipol, clofibrate, gemfibrozil, lovastatin, pravastatin, simvastatin, probucol or dextrothyroxine.

In another embodiment, a compound of the present invention may be administered in combination with more than one of the above-mentioned compounds, e.g. in combination with:

metformin and a sulfonylurea such as glyburide; a sulfonylurea and acarbose; nateglinide and metformin; acarbose and metformin; a sulfonylurea, metformin and troglitazone; insulin and a sulfonylurea; insulin and metformin; insulin, metformin and a sulfonylurea; insulin and troglitazone; insulin and lovastatin; etc.

In one embodiment, a compound of the present invention may be administered in combination with one or more antiobesity agents or appetite regulating agents. Such agents may be selected from the group consisting of CART (cocaine amphetamine regulated transcript) agonists, NPY (neuropeptide Y) antagonists, MC3 (melanocortin 3) agonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, β3 adrenergic agonists such as CL-316243, AJ-9677, GW-0604, LY362884, LY377267 or AZ-40140, MSH (melanocyte-stimulating hormone) agonists, MCH (melanocyte-concentrating hormone) antagonists, CCK (cholecystokinin) agonists, serotonin reuptake inhibitors (fluoxetine, seroxat or citalopram), norepinephrine reuptake inhibitors (e.g. sibutramine), 5HT (serotonin) agonists, bombesin agonists, galanin antagonists, growth hormone, growth factors such as prolactin or placental lactogen, growth hormone releasing compounds, TRH (thyreotropin releasing hormone) agonists, UCP 2 or 3 (uncoupling protein 2 or 3) modulators, leptin agonists, DA (dopamine) agonists (bromocriptin, doprexin), lipase/amylase inhibitors, PPAR modulators, RXR modulators, TR β agonists, adrenergic CNS stimulating agents, AGRP (agouti related protein) inhibitors, H3 histamine antagonists such as those disclosed in WO 00/42023, WO 00/63208 and WO 00/64884, the contents of all of which are incorporated herein by reference, exendin-4, GLP-1 agonists and ciliary neurotrophic factor. Further agents of relevance are bupropion (antidepressant), topiramate (anticonvulsant), ecopipam (dopamine D1/D5 antagonist), naltrexone (opioid antagonist), and peptide YY₃₋₃₆ (Batterham et al, Nature 418, 650-654 (2002)).

In one embodiment, the antiobesity agent employed is leptin.

In another embodiment, the antiobesity agent employed is a lipase inhibitor, e.g. orlistat.

In a further embodiment, the antiobesity agent employed is an adrenergic CNS-stimulating agent, e.g. dexamphetamine, amphetamine, phentermine, mazindol, phendimetrazine, diethylpropion, fenfluramine or dexfenfluramine.

In another embodiment, a compound of the present invention may be administered in combination with one or more antihypertensive agents. Examples of relevant antihypertensive agents are: β-blockers such as alprenolol, atenolol, timolol, pindolol, propranolol and metoprolol; ACE (angiotensin converting enzyme) inhibitors such as benazepril, captopril, enalapril, fosinopril, lisinopril, quinapril and ramipril; calcium channel blockers such as nifedipine, felodipine, nicardipine, isradipine, nimodipine, diltiazem and verapamil; and α-blockers such as doxazosin, urapidil, prazosin and terazosin.

It should be understood that treatment of a subject in need thereof with any suitable combination of a compound according to the invention with diet and/or exercise and/or with one or more of the above-mentioned compounds, and optionally with one or more other active substances is considered to be within the scope of the present invention.

The present invention also provides pharmaceutical compositions comprising as an active ingredient, at least one compound of the present invention, preferably in a therapeutically effective amount, suitable for use in any of the methods according to the present invention, together with one or more pharmaceutically acceptable carriers or excipients. Such pharmaceutical compositions may further comprise any of the further therapeutically active compounds as indicated above.

The pharmaceutical composition is preferably in unit dosage form, comprising from about 0.05 mg to about 1000 mg, preferably from about 0.1 mg to about 500 mg, and most preferably from about 0.5 mg to about 200 mg of a compound suitable for any of the methods described above.

### Pharmaceutical Compositions

The compounds of the present invention may be administered alone or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 20^{th} Edition, Gennaro, Ed., Mack Publishing Co., Easton, Pa., 2000.

The pharmaceutical composition may be specifically formulated for administration by any suitable route, such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen.

Pharmaceutical compositions for oral administration include solid dosage forms such as hard or soft capsules, tablets, troches, dragees, pills, lozenges, powders and granules. Where appropriate, they can be prepared with coatings such as enteric coatings, or they can be formulated so as to provide controlled release of the active ingredient, such as sustained or prolonged release, according to methods well known in the art.

Liquid dosage forms for oral administration include solutions, emulsions, aqueous or oily suspensions, syrups and elixirs.

Pharmaceutical compositions for parenteral administration include sterile aqueous and non-aqueous injectable solutions, dispersions, suspensions or emulsions, as well as sterile powders to be reconstituted in sterile injectable solutions or dispersions prior to use. Depot injectable formulations are also regarded as being within the scope of the present invention.

Other suitable administration forms include suppositories, sprays, ointments, crèmes, gels, inhalants, dermal patches, implants, etc.

A typical oral dosage is in the range of from about 0.001 to about 100 mg/kg body weight per day, preferably from about 0.01 to about 50 mg/kg body weight per day, and more preferably from about 0.05 to about 10 mg/kg body weight per day, administered in one or more doses such as 1-3 doses. The exact dosage will depend upon the frequency and mode of administration, the sex, age, weight and general condition of the subject treated, the nature and severity of the condition treated and any concomitant diseases to be treated, and other factors evident to those skilled in the art.

The formulations may conveniently be prepared in unit dosage form by methods known to those skilled in the art. A typical unit dosage form for oral administration one or more times per day, such as 1-3 times per day, may contain from 0.05 to about 1000 mg, preferably from about 0.1 to about 500 mg, and more preferably from about 0.5 mg to about 200 mg of a compound of the invention.

For parenteral routes such as intravenous, intrathecal, intramuscular and similar administration, typical dosages are in the order of about half the dosage employed for oral administration.

A compound for use according to the present invention is generally utilized as the free substance or as a pharmaceutically acceptable salt thereof. Examples of the latter are: an acid addition salt of a compound having a free base functionality, and a base addition salt of a compound having a free acid functionality. The term “pharmaceutically acceptable salt” refers to a non-toxic salt of a compound for use according to the present invention, which salts are generally prepared by reacting a free base with a suitable organic or inorganic acid, or by reacting an acid with a suitable organic or inorganic base. When a compound for use according to the present invention contains a free base functionality, such salts are prepared in a conventional manner by treating a solution or suspension of the compound with a chemical equivalent of a pharmaceutically acceptable acid. When a compound for use according to the present invention contains a free acid functionality, such salts are prepared in a conventional manner by treating a solution or suspension of the compound with a chemical equivalent of a pharmaceutically acceptable base. Physiologically acceptable salts of a compound with a hydroxy group include the anionic form of the compound in combination with a suitable cation, such as sodium or ammonium ion. Other salts which are not pharmaceutically acceptable may be useful in the preparation of compounds of the invention, and these form a further aspect of the invention.

For parenteral administration, solutions of compounds for use according to the present invention in sterile aqueous solution, in aqueous propylene glycol or in sesame or peanut oil may be employed. Aqueous solutions should be suitably buffered where appropriate, and the liquid diluent rendered isotonic with, e.g., sufficient saline or glucose. Aqueous solutions are particularly suitable for intravenous, intramuscular, subcutaneous and intraperitoneal administration. The sterile aqueous media to be employed are all readily available by standard techniques known to those skilled in the art.

Suitable pharmaceutical carriers include inert solid diluents or fillers, sterile aqueous solutions and various organic solvents. Examples of solid carriers are lactose, terra alba, sucrose, cyclodextrin, talc, gelatine, agar, pectin, acacia, magnesium stearate, stearic acid and lower alkyl ethers of cellulose. Examples of liquid carriers are syrup, peanut oil, olive oil, phospholipids, fatty acids, fatty acid amines, polyoxyethylene and water. Moreover, the carrier or diluent may include any sustained release material known in the art, such as glyceryl monostearate or glyceryl distearate, alone or mixed with a wax. The pharmaceutical compositions formed by combining the compounds for use according to the present invention and the pharmaceutically acceptable carriers are then readily administered in a variety of dosage forms suitable for the disclosed routes of administration. The formulations may conveniently be presented in unit dosage form by methods known in the art of pharmacy.

Formulations of the present invention suitable for oral administration may be presented as discrete units, such as capsules or tablets, which each contain a predetermined amount of the active ingredient, and which may include a suitable excipient. Furthermore, the orally available formulations may be in the form of a powder or granules, a solution or suspension in an aqueous or non-aqueous liquid, or an oil-in-water or water-in-oil liquid emulsion.

Compositions intended for oral use may be prepared according to any known method, and such compositions may contain one or more agents selected from the group consisting of sweetening agents, flavouring agents, colouring agents and preserving agents in order to provide pharmaceutically elegant and palatable preparations. Tablets may contain the active ingredient(s) in admixture with non-toxic pharmaceutically acceptable excipients which are suitable for the manufacture of tablets. These excipients may, for example, be: inert diluents, such as calcium carbonate, sodium carbonate, lactose, calcium phosphate or sodium phosphate; granulating and disintegrating agents, for example corn starch or alginic acid; binding agents, for example, starch, gelatine or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in U.S. Pat. Nos. 4,356,108; 4,166,452; and 4,265,874, the contents of which are incorporated herein by reference, to form osmotic therapeutic tablets for controlled release.

Formulations for oral use may also be presented as hard gelatine capsules where the active ingredient is mixed with an inert solid diluent, for example, calcium carbonate, calcium phosphate or kaolin, or a soft gelatine capsules wherein the active ingredient is mixed with water or an oil medium, for example peanut oil, liquid paraffin, or olive oil.

Aqueous suspensions may contain the compound for use according to the present invention in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more colouring agents, one or more flavouring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as a liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example, sweetening, flavouring, and colouring agents may also be present.

The pharmaceutical compositions comprising compounds for use according to the present invention may also be in the form of oil-in-water emulsions. The oily phase may be a vegetable oil, for example, olive oil or arachis oil, or a mineral oil, for example a liquid paraffin, or a mixture thereof. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavouring and colouring agents. The pharmaceutical compositions may be in the form of a sterile injectable aqueous or oleaginous suspension. This suspension may be formulated according to the known methods using suitable dispersing or wetting agents and suspending agents described above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution, and isotonic sodium chloride solution. In addition, sterile, fixed oils are conveniently employed as solvent or suspending medium. For this purpose, any bland fixed oil may be employed using synthetic mono- or diglycerides. In addition, fatty acids such as oleic acid find use in the preparation of injectables.

The compositions may also be in the form of suppositories for rectal administration of the compounds of the invention. These compositions can be prepared by mixing the drug with a suitable non-irritating excipient which is solid at ordinary temperatures but liquid at the rectal temperature and will thus melt in the rectum to release the drug. Such materials include, for example, cocoa butter and polyethylene glycols.

For topical use, creams, ointments, jellies, solutions of suspensions, etc., containing the compounds of the invention may be employed. In the context of the present invention, formulations for topical application include mouth washes and gargles.

Compounds of the present invention may also be administered in the form of liposome delivery systems, such as small unilamellar vesicles, large unilamellar vesicles, and multilamellar vesicles. Liposomes may be formed from a variety of phospholipids, such as cholesterol, stearylamine, or phosphatidylcholines.

In addition, some compounds of the present invention may form solvates with water or common organic solvents. Such solvates are also encompassed within the scope of the invention.

Thus, a further embodiment provides a pharmaceutical composition comprising a compound for use according to the present invention, or a pharmaceutically acceptable salt, solvate, or prodrug thereof, and one or more pharmaceutically acceptable carriers, excipients, or diluents.

If a solid carrier is used for oral administration, the preparation may be tabletted, placed in a hard gelatine capsule in powder or pellet form, or may be in the form of a troche or lozenge. The amount of solid carrier will vary widely, but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

A typical tablet that may be prepared by conventional tabletting techniques may contain:

| Core: | | |
|---|---|---|
| Active compound (as free compound or salt thereof) | 5.0 | mg |
| Lactosum Ph. Eur. | 67.8 | mg |
| Cellulose, microcryst. (Avicel) | 31.4 | mg |
| Amberlite® IRP88* | 1.0 | mg |
| Magnesii stearas Ph. Eur. | q.s. | |
| Coating: | | |
| Hydroxypropyl methylcellulose | approx. 9 | mg |
| Mywacett 9-40 T** | approx. 0.9 | mg |

*Polacrillin potassium NF, tablet disintegrant, Rohm and Haas.
**Acylated monoglyceride used as plasticizer for film coating.

If desired, the pharmaceutical composition comprising a compound according to the present invention may comprise a compound according to the present invention in combination with further active substances, such as those described in the foregoing.

The present invention also provides methods for the preparation of compounds for use according to the present invention. The compounds can be prepared readily according to the following general procedures (in which all variables are as defined before, unless so specified) using readily available starting materials, reagents and conventional synthesis procedures. In these reactions, it is also possible to make use of variants which are themselves known to those of ordinary skill in this art, but which are not mentioned in greater detail.

### EXAMPLES

### HPLC-MS (Method A)

A Hewlett Packard series 1100 instrument is used. The HPLC pump is connected to two eluent reservoirs containing: (A) 0.01% TFA in water, (B) 0.01% TFA in acetonitrile. Gradient: 5%-100% acetonitrile linear during 7.5 min at 1.5 ml/min. The analysis is performed at 40° C. by injecting an appropriate volume of the sample (preferably 1 μl) onto the column which is eluted with a gradient of acetonitrile. Detection: 210 nm, analogue output from DAD (diode array detector), ELS (analogue output from ELS), and MS ionisation mode API-ES, Scan 100-1000 amu step 0.1 amu. After the DAD the flow is divided yielding approx 1 ml/min to the ELS and 0.5 ml/min to the MS.

### HPLC-MS (Method B)

As Method A, except that the gradient is: 5%-100% acetonitrile linear during 4 min at 2.7 ml/min.

### Preparative HPLC-MS (Method C)

An Agilent series 1100 instrument is used. The HPLC pump is connected to two eluent reservoirs containing: (A) 0.1% TFA in water, (B) acetonitrile. Gradient: 60%-95% acetonitrile linear during 8 min at 10 ml/min. The analysis is performed at room temperature by injecting an appropriate volume of the sample onto the column (Luna 5μ C18(2) 100 Å, 250×10 mm) which is eluted with a gradient of acetonitrile. Detection: 210 nm, analogue output from DAD (diode array detector), ELS (analogue output from ELS), and MS ionisation mode API-ES, Scan 150-700 amu step 0.1 amu. After the DAD the flow is divided yielding approx 0.5 ml/min to the ELS and 0.5 ml/min to the MS. The mobile phase containing the desired molecular weight is automatically collected by the fraction collector.

### Preparative Procedures:

### General Procedure (A)

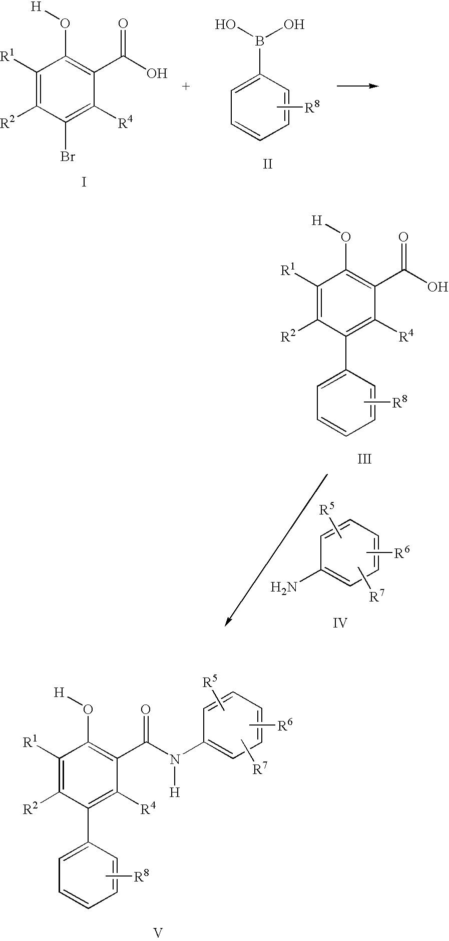

### Step A:

To a solution of the bromo-substituted phenol I (1 equivalent) in water the appropriate substituted aryl boronic acid II (1.1 equivalent) is added. The appropriate palladium catalyst (palladium acetate 0.005 to 0.01 equivalents) is added together with the appropriate base (sodium carbonate, 3 equivalents). The reaction mixture is stirred at room temperature overnight or heated under reflux overnight. The reaction is followed by TLC or LC-MS. The reaction mixture is made acidic by addition of 1N hydrochloric acid solution, and the mixture is stirred at room temperature for 1-3 hours. The water phase is extracted with ethyl acetate. The organic phase is dried over sodium sulfate, and the solvent is evaporated. Pure compounds III are obtained by crystallization from an organic solvent or by column chromatography.

The bromo-substituted phenols I are either commercially available or may be synthesized in one-step reactions from commercially available compounds by methods analogous to standard methods reported in the literature, e.g. Huang; Yunsheng et al; J. Med. Chem. 2001, 44 (11), 1815-1826, or Mach; Robert H et al; Med. Chem. Res. 1999, 9 (6) 355-373.

### Step B:

Compound III (1 equivalent) is dissolved in xylene together with the appropriate substituted aniline IV (1-1.1 equivalent). The reaction mixture is heated to reflux, and phosphorus trichloride, PCl₃ (0.33 equivalent) is carefully added. The reaction mixture is heated under reflux for 1-2 days. The reaction mixture is allowed to cool to room temperature, and the product V is isolated by filtration and purified by recrystallization from an organic solvent, or by column chromatography.

### General Procedure (B)

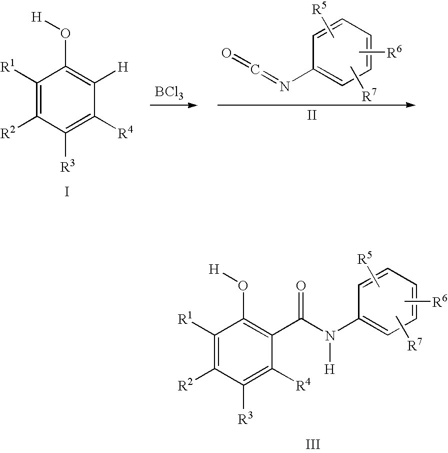

Boron trichloride catalyzed ortho carbamoylation of phenols may be achieved by a method similar to that described by O. Piccolo, L. Filippini, L. Tinucci, Tetrahedron, 1986, 42 (3), 885-892, as follows: A solution of the phenol I (1 mmol) in dichloromethane (5 ml) is added to a stirred solution of boron trichloride (1M in dichloromethane, 1-1.1 mmol) under nitrogen at −10° C. After 5 minutes, a solution of the aryl isocyanate II (1-1.1 mmol) in dichloromethane (5 ml) is added. The resulting mixture is stirred under reflux for 2 h, then at ˜40° C. overnight. The mixture is cooled to room temperature and stirred for 4 h after the addition of 4 M HCl (10 ml), and diethyl ether (25 ml) is then added. If a precipitate forms it is filtered off, rinsed with a small amount of diethyl ether and dried to give the anilide III.

If no well-defined precipitate forms, the organic layer is separated, dried, and the solvent is evaporated. The crude product may be purified by column chromatography.

The starting phenols I are either commercially available or may be synthesized in one-step reactions from commercially available compounds by methods analogous to standard methods reported in the literature, e.g. C. D. Braddock, S. C. Tucker, J. M. Brown, Bull. Soc. Chim. Fr. 1997, 134 (3-4) 399-410; B. P. Bandgar, L. S. Uppalla, V. S. Sadavarte, J. Chem. Research (S), 2000, 582-583; K. Menting, W. Eichel, K. Reimenschneider, H. L. K. Schmand, P. Boldt. J. Org. Chem. 1983, 48 (17) 2814-2820; Charpentier, Bruno; Bernardon, Jean-Michel; Eustache, Jacques; Millois, Corinne; Martin, Bernard; et al.; J. Med. Chem. 1995, 38 (26) 4993-5006. Thus, the introduction of R¹=tertiary alkyl or cycloalkyl may be accomplished starting from the appropriate phenol I with R¹═H by alkylation procedures, e.g. by treatment of the phenol with an alkylating agent in the presence of an acid catalyst. Suitable alkylating agents and catalysts are, e.g., halides such as R¹Cland a Lewis acid such as AlCl₃; or tertiary alcohols R¹OH in the presence of a strong acid such as H₂SO₄ or CF₃SO₃H.

Aryl isocyanates II may be prepared from the appropriate substituted anilines by treatment with phosgene or a phosgene equivalent as reported in the literature [see, e.g., K. Kurita, T. Matsumura, Y. Iwakura, J. Org. Chem. 1976, 41 (11) 2070-2071]. In order to suppress the formation of side-products such as 1,3-disubstituted urea and 1,3,5-trisubstituted biuret, it may be feasible to use the hydrochloride of the aniline rather than the aniline itself.

### General Procedure (C)

As for General procedure (B) except that the phenol I and the isocyanate II are dissolved in dry toluene instead of dichloromethane, and that the reaction mixture after the addition of the isocyanate is heated at 100-120° C. overnight.

### General Procedure (D)

As for General procedure (A) except that in Step B, compound III (1 mmol) is added to thionyl chloride (1-5 ml) and the mixture is stirred for 5-10 min. Acetonitrile (1-5 ml) is then added, followed by the appropriately substituted aniline IV (1.0-1.1 mmol). The mixture is stirred at room temperature for 30-90 min and then evaporated to dryness. The residue is stirred in 1 N NaOH (25 ml) for 15 minutes and acidified by the addition of 1 M HCl. The product V is isolated by filtration, washed with water and purified by crystallization from an organic solvent, or by column chromatography.

### General Procedure (E)

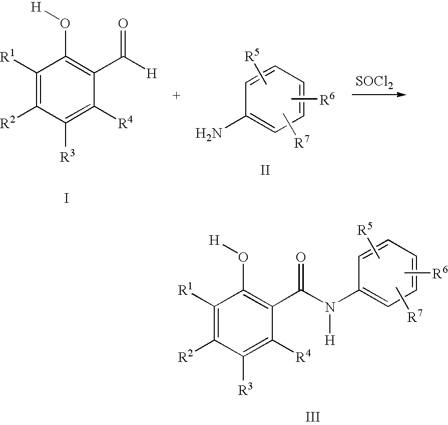

Compound I (1 equivalent) is added to excess thionyl chloride (2-20 equivalents) and the mixture is stirred for 5-10 min. Acetonitrile is added, followed by the appropriately substituted aniline II (1.0-1.1 equivalent). The mixture is stirred at room temperature for 30-90 min and then evaporated to dryness. The residue is either subjected to column chromatography to give compound III or stirred in 1 N NaOH (25 ml) for 15 minutes and acidified by the addition of 1 M HCl. Product III is then isolated by filtration, washed with water and purified by crystallization from an organic solvent, or by column chromatography.

### General Procedure (F)

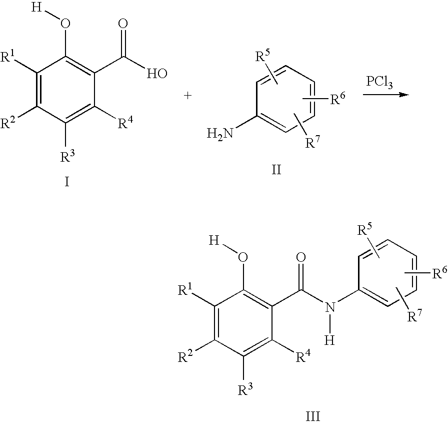

A mixture of compound I (1 equivalent) and the appropriately substituted aniline II (1.0-1.1 equivalent) in xylene is heated to reflux, and phosphorus trichloride (PCl₃) (0.33-1.0 equivalent) is carefully added. The reaction mixture is heated under reflux for 2-48 hours and allowed to cool to room temperature. The crude product is isolated by filtration or by evaporation to dryness, and pure compound III is obtained by recrystallization from an organic solvent, or by column chromatography.

### General Procedure (G)

As for General procedure (A) except that in Step B a mixture of compound III (1 mmol) and the appropriately substituted aniline (1.0-1.1 mmol) in acetonitrile (1-5 ml) thionyl chloride (1.5 mmol) is added. The mixture (suspension or solution) is stirred at room temperature for 5-10 hours. The compound is isolated by filtration and crystallization or by aqueous work-up: The residue is stirred in 1 N NaOH (25 ml) for 15 minutes and acidified by the addition of 1 M HCl. The product V is isolated by filtration or by extraction, washed with water, and purified by crystallization from an organic solvent, or by column chromatography.

### General Procedure (H)

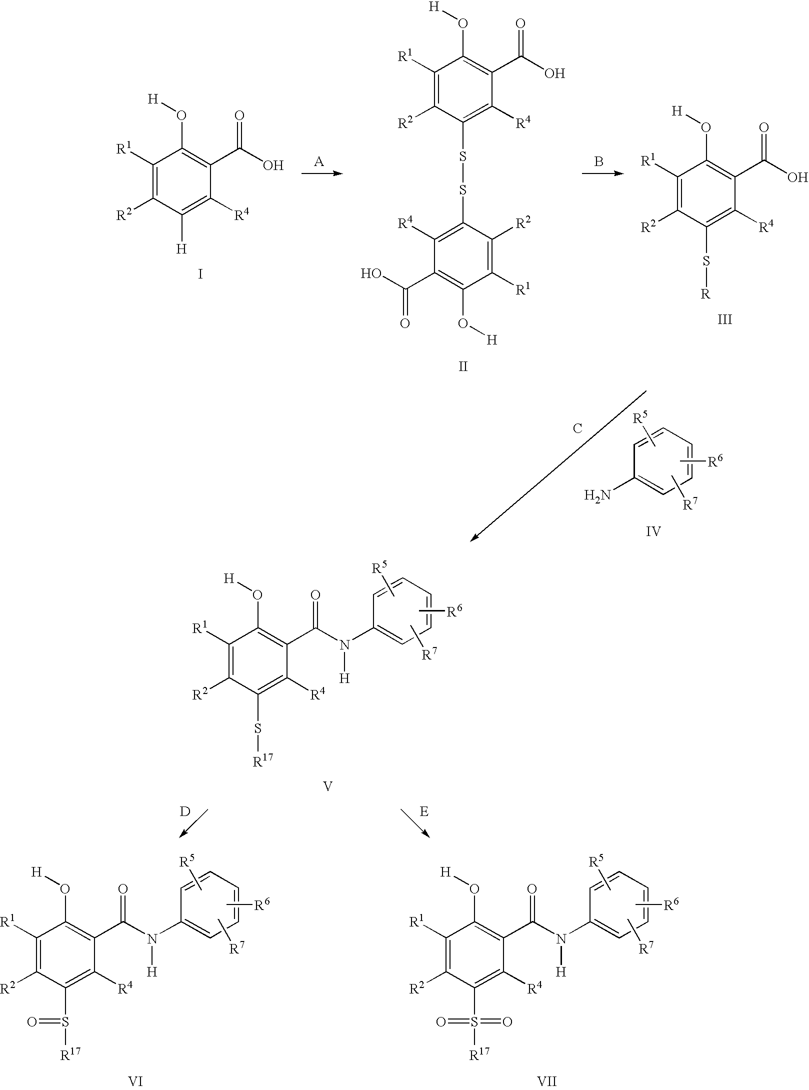

### Step A

To a suspension of compound I (10.0 mmol) in dichloromethane (20 ml) is added disulfur dichloride (5.0 mmol) dropwise. Acetonitrile (10 ml) is added, and a clear solution is formed. The reaction mixture is stirred overnight at room temperature. The separated compound II is isolated by filtration and used in the next reaction step without further purification.

### Step B:

To a solution of compound II (4.23 mmol) in methanol (45 ml) is added the appropriate alkylation reagent (12-42 mmol). Sodium borohydride (60.0 mmol) is added at a rate such that the temperature does not exceed 40° C.

Compound III is purified by aqueous acid work up, followed by crystallization or column chromatography.

### Step C:

This reaction is performed in a manner analogous to that in general procedure A (step B), general procedure D or general procedure E, starting from compound III obtained in step B above and the appropriately substituted aniline IV, to give compound V.

### Step D:

Compound V is oxidised using an appropriate oxidising reagent, such as hydrogen peroxide in acetic acid (stirring at room temperature for 2-12 hours), or m-chloro-peroxybenzoic acid (1 equivalent) in dichloromethane. Compound VI thus obtained is purified by filtration and crystallization, or by aqueous work-up followed by crystallization or column chromatography.

### Step E:

Compound V is oxidised using an appropriate oxidising reagent, such as hydrogen peroxide in acetic acid (heating at 100° C. for 6-12 hours), or m-chloro-peroxybenzoic acid (2 equivalents) in dichloromethane. Compound VI thus obtained is purified by filtration and crystallization, or by aqueous work-up followed by crystallization or column chromatography.

### Example 1

### General Procedure (A)

### 5-tert-Butyl-3′,5′-difluoro-4-hydroxy-2-methyl-biphenyl-3-carboxylic acid (2-chloro-4-trifluoromethanesulfonyl-phenyl)-amide

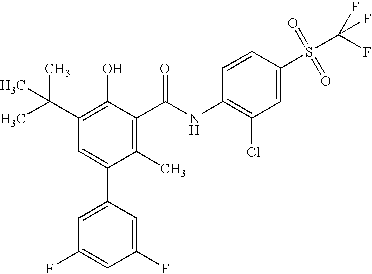

Step A: From 3-bromo-5-tert-butyl-6-hydroxy-2-methyl-benzoic acid and 3,5-difluoro phenyl-boronic acid.

Step B: From the product formed in step A and 2-chloro-4-trifluoromethanesulfonyl aniline

¹H NMR (DMSO-d₆): δ 1.40 (s, 9H) 2.18 (s, 3H) 6.99-7.07 (m, 2H) 7.12 (s, 1H) 7.19-7.27 (m, 1H) 8.20 (d, J=8.59 Hz, 1H) 8.24 (s, 1H) 8.64 (d, J=8.59 Hz, 1H) 9.16 (s, 1H) 10.61 (s, 1H); HPLC-MS (Method B): m/z=528, 530 (M+1); Rₜ=2.88 min.

### Example 2

### General Procedure (A)

### 5-tert-Butyl-4-hydroxy-2-methyl-4′-trifluoromethoxy-biphenyl-3-carboxylic acid (2-chloro-4-trifluoromethanesulfonyl-phenyl)-amide

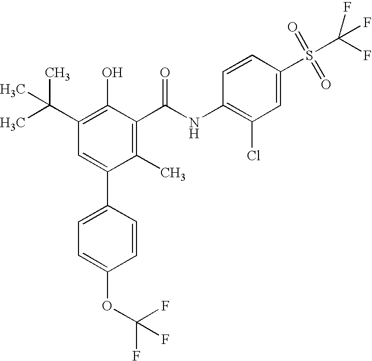

Step A: From 3-bromo-5-tert-butyl-6-hydroxy-2-methyl-benzoic acid and 4-trifluoromethoxy phenyl-boronic acid.

Step B: From the product formed in step A and 2-chloro-4-trifluoromethanesulfonyl aniline

¹H NMR (DMSO-d₆): δ 1.40 (s, 9H) 2.17 (s, 3H) 7.11 (s, 1H) 7.39-7.46 (m, 4H) 8.20 (d, J=8.59 Hz, 1H) 8.25 (d, J=2.02 Hz, 1H) 8.65 (d, 1H) 9.09 (s, 1H) 10.65 (s, 1H); HPLC-MS (Method B): m/z=2.97 (M+1); Rₜ=6.10 min.

### Example 3

### General Procedure (A)

### 5-tert-Butyl-4-hydroxy-2-methyl-3′-trifluoromethyl-biphenyl-3-carboxylic acid (2-chloro-4-trifluoromethanesulfonyl-phenyl)-amide

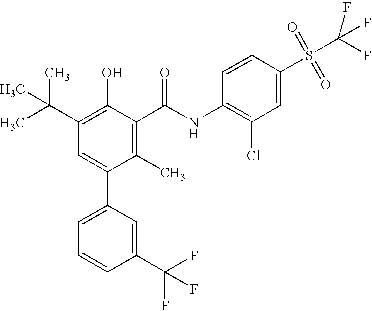

Step A: From 3-bromo-5-tert-butyl-6-hydroxy-2-methyl-benzoic acid and 3-trifluoromethyl phenyl-boronic acid.

Step B: From the product formed in step A and 2-chloro-4-trifluoromethanesulfonyl aniline

¹H NMR (DMSO-d₆): δ 1.41 (s, 9H) 2.17 (s, 3H) 7.13 (s, 1H) 7.58-7.77 (m, 4H) 8.21 (d, J=8.59 Hz, 1H) 8.25 (s, 1H) 8.65 (d, J=8.59 Hz, 1H) 9.14 (s, 1H) 10.62 (s, 1H); HPLC-MS (Method B): m/z=594 (M+1); Rₜ=2.92 min.

### Example 4

### General Procedure (A)

### 3-Bromo-5-tert-butyl-N-(2-chloro-4-trifluoromethanesulfonyl-phenyl)-6-hydroxy-2-methyl-benzamide

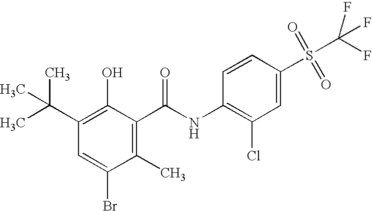

Step B: From 3-bromo-5-tert-butyl-6-hydroxy-2-methyl-benzoic acid and 2-chloro-4-trifluoromethanesulfonyl aniline

¹H NMR (DMSO-d₆): δ 1.37 (s, 9H) 2.29 (s, 3H) 7.39 (s, 1H) 8.20 (d, J=8.59 Hz, 1H) 8.25 (s, 1H) 8.61 (d, J=8.59 Hz, 1H) 9.21 (s, 1H) 10.69 (s, 1H); HPLC-MS (Method B): m/z=528, 530, 532 (M+1); Rₜ=2.78 min.

### Example 5

### General Procedure (A)

### 5-tert-Butyl-4′-fluoro-4-hydroxy-2-methyl-biphenyl-3-carboxylic acid (2-chloro-4-trifluoromethanesulfonyl-phenyl)-amide

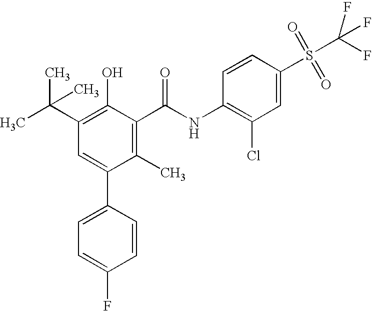

Step A: From 3-bromo-5-tert-butyl-6-hydroxy-2-methyl-benzoic acid and 4-fluoro phenyl-boronic acid.

Step B: From the product formed in step A and 2-chloro-4-trifluoromethanesulfonyl aniline

¹H NMR (DMSO-d₆): δ 1.39 (s, 9H) 2.15 (s, 3H) 7.08 (s, 1H) 7.26 (t, J=8.84 Hz, 2H) 7.30-7.39 (m, 2H) 8.20 (d, J=9.10 Hz, 1H) 8.24 (s, 1H) 8.63 (d, J=8.59 Hz, 1H) 9.02 (s, 1H) 10.62 (s, 1H); HPLC-MS (Method B): m/z=544, 546 (M+1); Rₜ=2.90 min.

### Example 6

### General Procedure (A)

### 5-tert-Butyl-4-hydroxy-2-methyl-4′-methylsulfanyl-biphenyl-3-carboxylic acid (2-chloro-4-trifluoromethanesulfonyl-phenyl)-amide

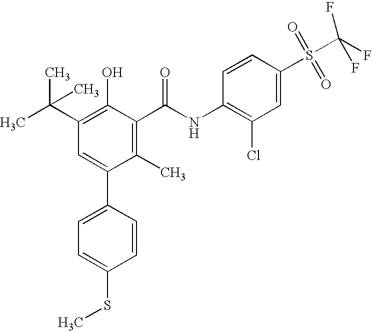

Step A: From 3-bromo-5-tert-butyl-6-hydroxy-2-methyl-benzoic acid and 4-methylthio phenyl-boronic acid.

Step B: From the product formed in step A and 2-chloro-4-trifluoromethanesulfonyl aniline

¹H NMR (DMSO-d₆): 1.47 (s, 9H) 2.17 (s, 3H) 2.49 (s, 3H) 7.08 (s, 1H) 7.25 (d, 2H) 7.33 (d, 2H) 8.20 (d, 1H) 8.24 (s, 1H) 8.62 (d, 1H) 8.99 (s, 1H) 10.60 (s, 1H); HPLC-MS (Method B): m/z=572, 574 (M+1); Rₜ=2.90 min.

### Example 7

### General Procedure (A)

### 5-tert-Butyl-3′,4′-difluoro-4-hydroxy-2-methyl-biphenyl-3-carboxylic acid (2-chloro-4-trifluoromethanesulfonyl-phenyl)-amide

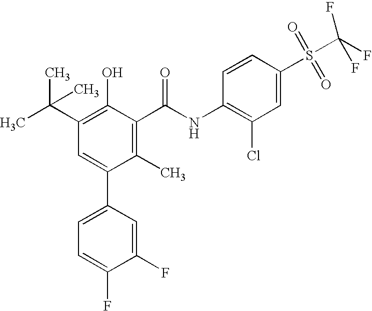

Step A: From 3-bromo-5-tert-butyl-6-hydroxy-2-methyl-benzoic acid and 3,4-difluoro phenyl-boronic acid.

Step B: From the product formed in step A and 2-chloro-4-trifluoromethanesulfonyl aniline

¹H NMR (DMSO-d₆): δ 1.39 (s, 9H) 2.16 (s, 3H) 7.10 (s, 1H) 7.11-7.19 (m, 1H) 7.33-7.43 (m, 1H) 7.43-7.55 (m, 1H) 8.20 (d, J=9.04 Hz, 1H) 8.25 (d, J=1.88 Hz, 1H) 8.63 (d, J=8.29 Hz, 1H) 9.09 (s, 1H) 10.60 (s, 1H); HPLC-MS (Method B): m/z=562, 564 (M+1); Rₜ=2.92 min.

### Example 8

### General Procedure (A)

### 5-tert-Butyl-4′-cyano-4-hydroxy-2-methyl-biphenyl-3-carboxylic acid (2-chloro-4-trifluoromethanesulfonyl-phenyl)-amide

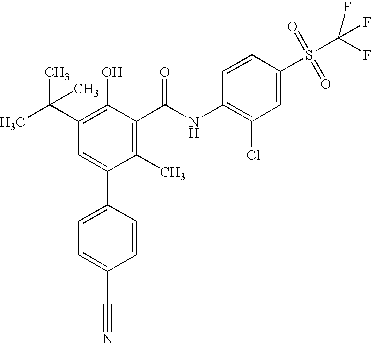

Step A: From 3-bromo-5-tert-butyl-6-hydroxy-2-methyl-benzoic acid and 4-cyano phenyl-boronic acid.

Step B: From the product formed in step A and 2-chloro-4-trifluoromethanesulfonyl aniline

¹H NMR (DMSO-d₆): δ ppm 1.40 (s, 9H) 2.17 (s, 3H) 7.11 (s, 1H) 7.53 (d, J=8.59 Hz, 2H) 7.90 (d, J=8.08 Hz, 2H) 8.20 (d, J=9.60 Hz, 1H) 8.24 (s, 1H) 8.64 (d, J=8.59 Hz, 1H) 9.19 (s, 1H) 10.65 (s, 1H); HPLC-MS (Method B): m/z=551, 553 (M+1); Rₜ=2.72 min.

### Example 9

### General Procedure (A)

### 3-Bromo-5-tert-butyl-N-(3-chloro-4-trifluoromethylsulfanyl-phenyl)-6-hydroxy-2-methyl-benzamide

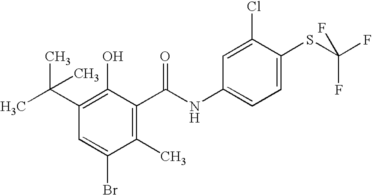

Step B: From 3-bromo-5-tert-butyl-6-hydroxy-2-methyl-benzoic acid and 3-chloro-4-trifluoromethanesulfanyl aniline.

¹H NMR (DMSO-d₆): δ 1.35 (s, 9H) 2.22 (s, 3H) 7.38 (s, 1H) 7.72 (dd, J=8.48, 2.07 Hz, 1H) 7.87 (d, J=8.67 Hz, 1H) 8.23 (d, J=1.88 Hz, 1H) 9.12 (s, 1H) 10.94 (s, 1H); HPLC-MS (Method B): m/z=496, 498, 500 (M+1); Rₜ=2.86 min.

### Example 10

### General Procedure (A)

### 5-tert-Butyl-3′,4′-difluoro-4-hydroxy-2-methyl-biphenyl-3-carboxylic acid (3-chloro-4-trifluoromethylsulfanyl-phenyl)-amide

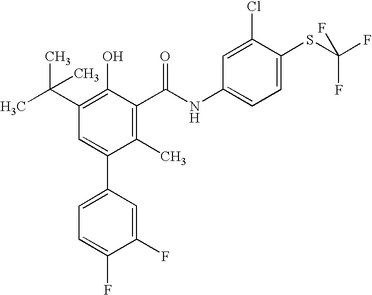

Step A: From 3-bromo-5-tert-butyl-6-hydroxy-2-methyl-benzoic acid and 3,4-difluoro phenyl-boronic acid.

Step B: From the product formed in step A and 3-chloro-4-trifluoromethanesulfanyl aniline

¹H NMR (DMSO-d₆): δ 1.38 (s, 9H) 2.08 (s, 3H) 7.08 (s, 1H) 7.11-7.19 (m, 1H) 7.30-7.42 (m, 1H) 7.42-7.56 (m, 1H) 7.75 (d, 1H) 7.86 (d, 1H) 8.26 (d, J=1.88 Hz, 1H) 8.98 (s, 1H) 10.95 (s, 1H); HPLC-MS (Method B): m/z=530, 532 (M+1); Rₜ=3.00 min.

### Example 11

### General Procedure (A)

### 5-tert-Butyl-3′-chloro-4′-fluoro-4-hydroxy-2-methyl-biphenyl-3-carboxylic acid (2-chloro-4-trifluoromethanesulfonyl-phenyl)-amide

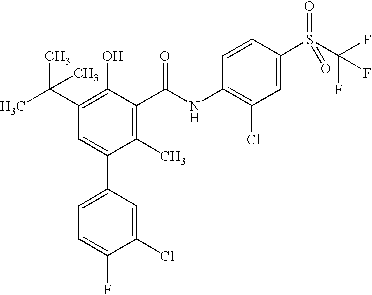

Step A: From 3-bromo-5-tert-butyl-6-hydroxy-2-methyl-benzoic acid and 3-chloro-4-fluoro phenyl-boronic acid.

Step B: From the product formed in step A and 2-chloro-4-trifluoromethanesulfonyl aniline

¹H NMR (DMSO-d₆): δ 1.40 (s, 9H) 2.16 (s, 3H) 7.10 (s, 1H) 7.26-7.35 (m, 1H) 7.45-7.54 (m, 2H) 8.20 (d, J=8.67 Hz, 1H) 8.25 (d, J=1.88 Hz, 1H) 8.63 (d, J=8.29 Hz, 1H) 9.10 (s, 1H) 10.59 (s, 1H); HPLC-MS (Method B): m/z=578, 580, 582 (M+1); Rₜ=2.99 min.

### Example 12

### General Procedure (A)

### 5-tert-Butyl-4′-fluoro-4-hydroxy-2,3′-dimethyl-biphenyl-3-carboxylic acid (2-chloro-4-trifluoromethanesulfonyl-phenyl)-amide

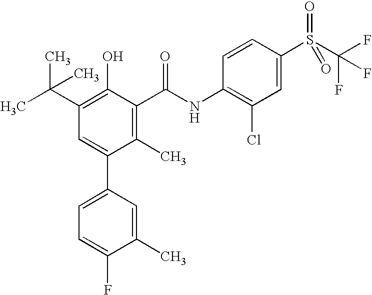

Step A: From 3-bromo-5-tert-butyl-6-hydroxy-2-methyl-benzoic acid and 3-methyl-4-fluoro phenyl-boronic acid.

Step B: From the product formed in step A and 2-chloro-4-trifluoromethanesulfonyl aniline

¹H NMR (DMSO-d₆): δ 1.39 (s, 9H) 2.15 (s, 3H) 2.26 (3H) 7.00 (d, J=8.67 Hz, 1H) 7.05-7.08 (m, 1H) 7.68 (dd, J=8.85, 2.07 Hz, 1H) 7.79 (d, J=2.26 Hz, 1H) 8.19 (dd, 1H) 8.25 (d, J=2.26 Hz, 1H) 8.62 (d, J=8.29 Hz, 1H) 10.60 (s, 1H); HPLC-MS (Method B): m/z=558, 560 (M+1); Rₜ=2.98 min.

### Example 13

### General Procedure (A)

### 3-Chloro-5-tert-butyl-N-(2-chloro-4-trifluoromethanesulfonyl-phenyl)-6-hydroxy-2-methyl-benzamide

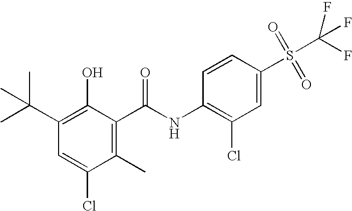

Step B: From 3-chloro-5-tert-butyl-6-hydroxy-2-methyl-benzoic acid and 2-chloro-4-trifluoromethanesulfonyl aniline

¹H NMR (DMSO-d₆): δ 1.36 (s, 9H) 2.27 (s, 3H) 7.24 (s, 1H) 8.20 (d, J=8.67 Hz, 1 H) 8.25 (d, J=1.88 Hz, 1H) 8.60 (d, J=8.67 Hz, 1H) 9.17 (s, 1H) 10.67 (s, 1H); HPLC-MS (Method B): m/z=484, 486, 488 (M+1); Rₜ=2.74 min.

### Example 14

### 5-tert-Butyl-4-hydroxy-4′-methanesulfonyl-2-methyl-biphenyl-3-carboxylic acid (2-chloro-4-trifluoromethanesulfonyl-phenyl)-amide

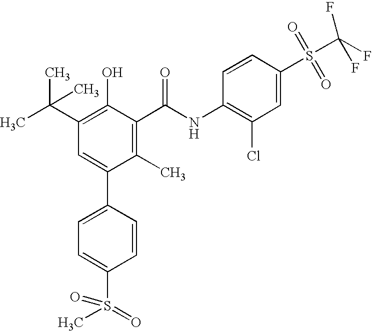

To a solution of 5-tert-Butyl-4-hydroxy-2-methyl-4′-methylsulfanyl-biphenyl-3-carboxylic acid (2-chloro-4-trifluoromethanesulfonyl-phenyl)-amide (1 equivalent) in dichloromethane 3-chloro-peroxybenzoic acid (2 equivalents) was added. The reaction mixture was stirred at room temperature overnight. The organic phase was washed with sodium carbonate solution and evaporated the title compound was purified by column chromatography.

¹H NMR (CDCl₃): δ 1.37 (s, 9H) 2.37 (s, 3H) 7.20 (s, 1H) 7.45 (d, J=8.29 Hz, 2H) 7.96 (d, J=8.29 Hz, 2H) 8.04 (d, J=1.88 Hz, 1H) 8.46 (s, 1H) 8.97 (d, J=9.04 Hz, 1H) 9.51 (s, 1H); HPLC-MS (Method B): m/z=626, 628 (M+1); Rₜ=2.52 min.

### Example 15

### 5-tert-Butyl-3′,4′-difluoro-4-hydroxy-2-methyl-biphenyl-3-carboxylic acid (3-chloro-4-trifluoromethanesulfinyl-phenyl)-amide

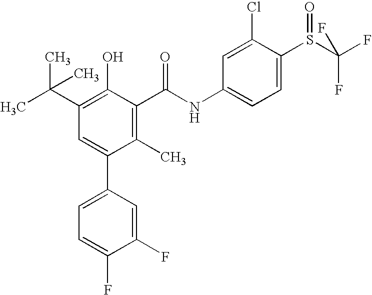

The compound was prepared by a method described in: Tetrahedron Letters, vol. 35, No 28, pp. 4955-4958, 1994. To a solution of 5-tert-Butyl-3′,4′-difluoro-4-hydroxy-2-methylbiphenyl-3-carboxylic acid (3-chloro-4-trifluoromethylsulfanyl-phenyl)-amide (1 equivalent) in a mixture of carbontetrachloride and acetonitril 1:1 sodium periodate (3 equivalents) and ruthenium trichloride hydrate (0.05 equivalents) were added. The reaction mixture was stirred at room temperature overnight. Water and diethyl ether was added and the organic phase was separated. The organic phase was washed with sodium carbonate solution and evaporated. The title compound was purified by column chromatography.

¹H NMR (CDCl₃): δ 1.37 (s, 9H) 2.37 (s, 3H) 7.20 (s, 1H) 7.45 (d, J=8.29 Hz, 2H) 7.96 (d, J=8.29 Hz, 2H) 8.04 (d, J=1.88 Hz, 1H) 8.46 (s, 1H) 8.97 (d, J=9.04 Hz, 1H) 9.51 (s, 1H); HPLC-MS (Method B): m/z=626, 628 (M+1); Rₜ=2.52 min.

### Example 16

### General Procedure (A) Step B

### 1-Hydroxy-naphthalene-2-carboxylic acid (3-trifluoromethanesulfonyl-phenyl)-amide

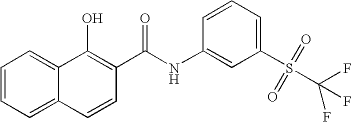

From 1-hydroxy-2-naphthoic acid and 3-trifluoromethanesulfonyl-aniline; mp 194-195° C.; ¹H NMR (DMSO-d₆): δ 13.47 (s, 1H), 10.95 (s, 1H), 8.62 (s, 1H), 8.46-8.41 (m, 1H), 8.33 (d, J=8.08 Hz, 1H), 8.11 (d, J=9.10 Hz, 1H), 7.97-7.87 (m, 3H), 7.73-7.67 (m, 1H), 7.64-7.58 (m, 1H), 7.52 (d, J=9.10 Hz, 1H); HPLC-MS (Method A): m/z: 396 (M+H), 418 (M+Na) Rₜ=5.1 min.

### Example 17

### General Procedure (A) Step B

### 1-Hydroxy-naphthalene-2-carboxylic acid (3-trifluoromethylsulfanyl-phenyl)-amide

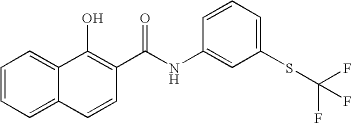

From 1-hydroxy-2-naphthoic acid and 3-trifluoromethanesulfanyl-aniline; white crystals, mp 154° C.; ¹H NMR (DMSO-d₆): δ 13.73 (s, 1H), 10.68 (s, 1H), 8.32 (d, J=8.08 Hz, 1H), 8.19 (br s, 1H), 8.12 (d, J=8.59 Hz, 1H), 8.04-8.00 (m, 1H), 7.93 (d, J=8.08 Hz, 1H), 7.72-7.66 (m, 1H), 7.63-7.52 (m, 3H), 7.50 (d, 1H); HPLC-MS (Method A): m/z: 364 (M+1), 386 (M+Na), Rₜ=5.4 min.

### Example 18

### General Procedure (A) Step B

### 2-Hydroxy-naphthalene-1-carboxylic acid (3-trifluoromethanesulfonyl-phenyl)-amide

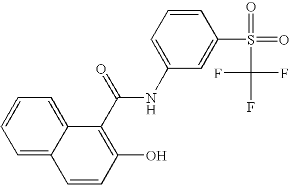

From 2-hydroxy-1-naphthoic acid and 3-trifluoromethanesulfonyl-aniline; mp 179-180° C.; ¹H NMR (DMSO-d₆): δ 8.74 (s, 1H), 8.18 (d, J=8.08 Hz, 1H), 7.91-7.71 (m, 5H), 7.51-7.43 (m, 1H), 7.36-7.29 (m, 1H), 7.19 (d, J=9.10 Hz, 1H); HPLC-MS (Method A): m/z: 396 (M+H), 418 (M+Na); Rₜ=4.0 min.

### Example 19

### 3-Hydroxy-naphthalene-2-carboxylic acid (3-trifluoromethanesulfonyl-phenyl)-amide

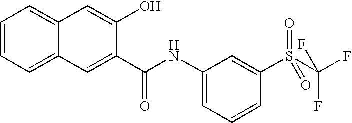

### Step A:

### 3-Methoxy-naphthalene-2-carboxylic acid (3-trifluoromethanesulfonyl-phenyl)-amide

The title compound was prepared from 3-methoxy-naphthalene-2-carboxylic acid, 3-trifluoromethanesulfonyl-aniline and PCl₃ in xylene according to the General procedure (A) Step B; white crystals; ¹H NMR (CDCl₃): δ 10.23 (s, 1H), 8.87 (s, 1H), 8.38-8.34 (br peak at 8.28 ppm, 1H), 7.94 (d, J=8.08 Hz, 1H), 7.82-7.76 (m, 2H), 7.69 (t, J=8.08 Hz, 1H), 7.61-7.55 (m, 1H), 7.48-7.42 (m, 1H), 7.32 (s, 1H), 4.21 (s, 3H); HPLC-MS (Method B): m/z: 410 (M+H), 432 (M+Na), 841 (2M+Na); Rₜ=2.39 min.

### Step B:

Boron tribromide (8.1 ml of a 0.1 M solution in dichloromethane) was added to a stirred solution of 3-methoxy-naphthalene-2-carboxylic acid (3-trifluoromethanesulfonylphenyl)-amide (0.33 g, 0.8 mmol) in dry dichloromethan (10 ml) at −70° C. under nitrogen. Then the dry ice bath was removed and the mixture was allowed to warm up to room temperature. After 30 min. the mixture was extracted with saturated aqueous sodium bicarbonate (2×30 ml). The organic phase was dried over MgSO₄, filtered and evaporated to give the title compound. Yield 0.32 g (100%); pale yellow crystals, mp 212-213° C.; ¹H NMR (DMSO-d₆): δ 11.05 (br s, 1H), 8.76-8.73 (br, peak at 8.75 ppm, 1H), 8.42 (br s, 1H), 8.30-8.25 (m, 1H), 7.93 (d, J=8.08 Hz, 1H), 7.90-7.84 (m, 2H), 7.78 (d, J=8.08 Hz, 1H), 7.55-7.49 (m, 1H), 7.40-7.32 (m, 2H); HPLC-MS (Method B): m/z: 396 (M+H), 418 (M+Na); Rₜ=2.29 min.

### Example 20

### General Procedure (B)

### 1-Hydroxy-5,6,7,8-tetrahydro-naphthalene-2-carboxylic acid (2-chloro-4-trifluoromethanesulfonyl-phenyl)-amide

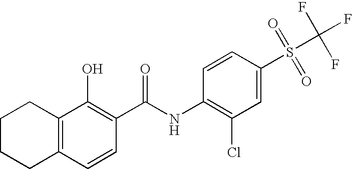

From 1,2,3,4-tetrahydro-5-naphthol and 2-chloro-1-isocyanato-4-trifluoromethanesulfonyl-benzene; white crystals, ¹H NMR (CDCl₃): δ d 11.73 (s, 1H), 8.92 (d, J=9.09 Hz, 1H), 8.10 (d, J=2.02 Hz, 1H), 7.99 (dd, 1H), 7.30 (d, J=8.08 Hz, 1H), 6.72 (d, J=8.08 Hz, 1H), 2.82 (m, 2H), 2.75-2.69 (m, 2H), 1.88-1.75 (m, 4H); HPLC-MS (Method B): m/z: 434 (M+H), 436 (M+2+H), 456 (M+Na), 458 (M+2+Na), Rₜ=2.75 min.

### Example 21

### General Procedure (B)

### 1-Hydroxy-5,6,7,8-tetrahydro-naphthalene-2-carboxylic acid (4-trifluoromethylsulfanyl-phenyl)-amide

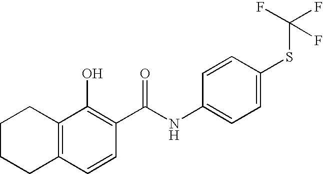

From 1,2,3,4-tetrahydro-5-naphthol and 1-isocyanato-4-trifluoromethanesulfanyl-benzene; white crystals, mp 167° C.; ¹H NMR (CDCl₃): δ d 12, 13 (s, 1H), 7.94 (s, 1H), 7.70-7.56 (m, 4H), 7.23 (d, J=8.08 Hz, 1H), 6.65 (d, J=8.08 Hz, 1H), 2.80-2.61 (br peaks at 2.76 & 2.70, 4H), 1.86-1.69 (br, 4H); HPLC-MS (Method A): m/z: 368 (M+H), 390 (M+Na), Rₜ=5.78 min.

### Example 22

### General Procedure (B)

### 4-Benzenesulfonyl-1-hydroxy-naphthalene-2-carboxylic acid (4-trifluoromethylsulfanyl-phenyl)-amide

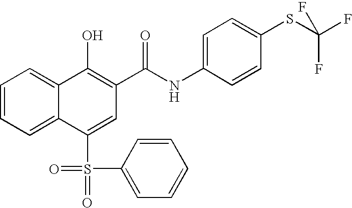

4-Benzenesulfonyl-1-hydroxy-naphthalene-2-carboxylic acid phenyl ester (0.210 g, 0.52 mmol) and 4-trifluoromethylsulfanyl-aniline (0.075 ml, 1 equivalent) was stirred in a round bottom flask together with dry toluene (1 ml). The mixture was heated at 165-175° C. for 45 min., allowing the toluene to evaporate. The residue was triturated with ether-pentane 1:1 (1 ml) and the precipitate was filtered off, rinsed on the filter with the same solvent and dried. The crude product was extracted with dichloromethane (2 ml) and the insoluble material was collected by filtration and dried to give the title compound as a white solid. Yield 0.139 g, mp 271-272° C. ¹H NMR (DMSO-d₆): δ 11.50 (s, 1H), 9.14 (s, 1H), 8.45-8.40 (br d-like, 2H), 8.06-8.00 (br d-like, 2H), 7.98-7.91 (br d-like, 2H), 7.83-7.74 (m, 3H), 7.69-7.56 (m, 5H); HPLC-MS (Method B): m/z: 504 (M+H), 526 (M+Na); Rₜ=2.86 min.

### Example 23

### General Procedure (B)

### 4-Chloro-1-hydroxy-naphthalene-2-carboxylic acid (4-trifluoromethylsulfanyl-phenyl)amide

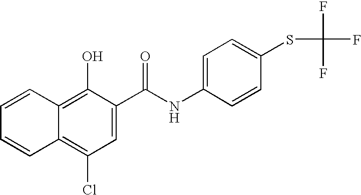

From 4-chloro-1-naphthol and 1-isocyanato-4-trifluoromethanesulfanyl-benzene; beige crystals, mp 172-175° C.; ¹H NMR (CDCl₃): δ d 13.27 (s, 1H), 8.51 (d, 1H), 8.20 (d, 1H), 7.94 (br s, 1H), 7.80-7.69 (m, 5H), 7.67-7.62 (m, 1H); HPLC-MS (Method B): m/z: 398 (M+H), 400 (M+2+H), Rₜ=2.98 min.

### Example 24

### General Procedure (B)

### 3-Adamantan-1-yl-N-(2-chloro-4-trifluoromethanesulfonyl-phenyl)-2-hydroxy-5-methyl-benzamide

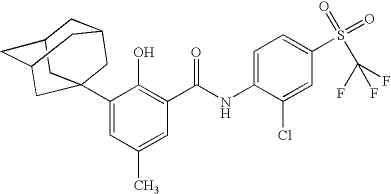

From 2-(1-adamantyl)-4-methylphenol and 2-chloro-1-isocyanato-4-trifluoromethanesulfonyl-benzene; pale yellow crystals, mp 203-204° C. and 209-211° C. (from heptane-EtOAc); ¹H NMR (DMSO-d₆): δ d 11.90 (s, 1H), 8.87 (d, 1H), 8.86 (s, 1H), 8.10 (d, 1H), 7.99 (dd, J=8.58 Hz, 2.03 Hz, 1H), 7.28 (s, 1H), 7.18 (s, 1H), 2.35 (s, 3H), 2.18-2.13 (br m, 6H), 2.13-2.06 (br s, 3H), 1.83-1.74 (br, 6H); HPLC-MS (Method B): m/z: 528 (M+H), 530 (M+2+H), 550 (M+Na), 552 (M+2+Na); Rₜ=3.29 min.

### Example 25

### General Procedure (B)

### 4-Chloro-1-hydroxy-naphthalene-2-carboxylic acid (2-chloro-4-trifluoromethanesulfonyl-phenyl)-amide

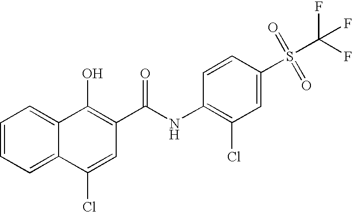

From 4-chloro-1-naphthol and 2-chloro-1-isocyanato-4-trifluoromethanesulfonyl-benzene; white crystals, mp 234° C.; HPLC-MS (Method B): m/z 432 (M+H), 434 (M+2+H), R=2.98; ¹H NMR (CDCl₃) J 12.01 (s, 1H), 8.76 (d, J=9.10 Hz, 1H), 8.50 (br d, 1H), 8.33 (d, J=2.02 Hz, 1H), 8.21 (s, 1H), 8.20 (dd, 1H), 8.16 (br d, 1H), 7.88-7.83 (m, 1H), 7.76-7.71 (m, 1H).

### Example 26

### General Procedure (B)

### 3-tert-Butyl-N-(2-chloro-4-trifluoromethanesulfonyl-phenyl)-2-hydroxy-5-methylbenzamide

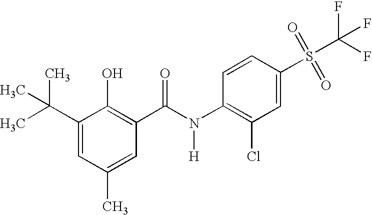

From 2-tert-butyl-4-methylphenol and 2-chloro-1-isocyanato-4-trifluoromethanesulfonyl-benzene; white crystals, mp 209-212° C.; ¹H NMR (CDCl₃): δ 11.87 (s, 1H), 8.89 (d, J=8.89 Hz, 1H), 8.86 (br s, 1H), 8.11 (d, J=2.02 Hz, 1H), 8.00 (dd, 1H), 7.34 (br d, 1H), 7.19 (br, 1H), 2.36 (s, 3H), 1.43 (s, 9H); HPLC-MS (Method B): m/z: 450 (M+H), 452 (M+2+H), Rₜ=3.00 min.

### Example 27

### General Procedure (B)

### 4-Chloro-1-hydroxy-naphthalene-2-carboxylic acid (3-chloro-4-trifluoromethylsulfanyl-phenyl)-amide

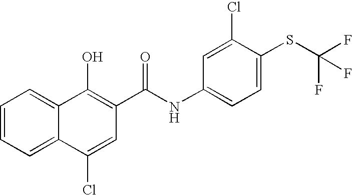

From 4-chloro-1-naphthol and 2-chloro-4-isocyanato-1-trifluoromethylsulfanyl-benzene; yellow crystals, mp 148-149° C.; HPLC-MS (Method B): m/z 432 (M+H), 434 (M+2+H), Rₜ=2.98; ¹H NMR (CDCl₃) δ 13.49 (s, 1H), 9.73 (br s, 1H), 8.52-8.46 (br d, 1H), 8.20-8.14 (br d, 1H), 8.10 (br s, 1H), 8.05 (br s-like (d)), 7.85-7.79 (d-like br m, 1H), 7.77-7.70 (d-like br m, 2H), 7.65-7.58 (t-like br m, 1H).

### Example 28

### General Procedure (B)

### 3-Adamantan-1-yl-N-(3-chloro-4-trifluoromethylsulfanyl-phenyl)-2-hydroxy-5-methylbenzamide

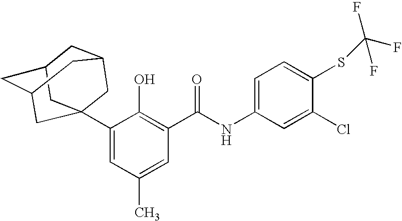

From 2-(1-adamantyl)-4-methylphenol and 2-chloro-4-isocyanato-1-trifluoromethylsulfanyl-benzene; HPLC-MS: m/z: 496 (M+H), 498 (M+2+H), 518 (M+Na), 520 (M+2+Na), Rₜ=3.42 min.

### Example 29

### General Procedure (B)

### 3-Adamantan-1-yl-N-(3-chloro-4-trifluoromethylsulfanyl-phenyl)-2-hydroxy-5-methylbenzamide

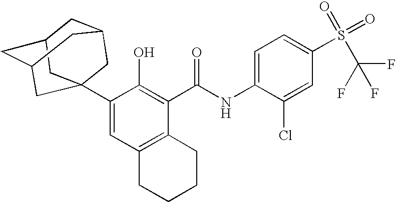

### Step A:

### 3-adamantan-1-yl-5,6,7,8-tetrahydro-naphthalen-2-ol

From 5,6,7,8-tetrahydro-2-naphthol and adamantan-1-ol in H₂SO₄/CH₂Cl₂; crystals with a purple tinge, mp 169-169.5° C. (from 96% EtOH); ¹H NMR (DMSO-d₆): δ 6.89 (s, 1H), 6.36 (s, 1H), 4.53 (s, 1H, OH), 2.71-2.62 (m, 4H), 2.15-2.03 (br m, peaks at 2.10 and 2.06, 6H+3H), 1.80-1.70 (m, 6H+4H).

### Step B:

From 3-adamantan-1-yl-5,6,7,8-tetrahydro-naphthalen-2-ol and 2-chloro-1-isocyanato-4-trifluoromethanesulfonyl-benzene; cream-colored crystals, mp 205-206° C.; HPLC-MS (Method B): m/z: 568 (M+H), 570 (M+2+H), 590 (M+Na), 592 (M+2+Na); Rₜ=3.24 min.; ¹H NMR (DMSO-d₆): δ 9.41 (s, 1H), 9.01 (d, J=8.59 Hz, 1H), 8.48 (br s, 1H), 8.08 (d, J=2.02 Hz, 1H), 7.99 (dd, 1H), 7.09 (s, 1H), 2.95-2.89 (m, 2H), 2.81-2.74 (m, 2H), 2.16-2.04 (m, peaks at 2.12 and 2.08, 6H+3H), 1.87-1.71 (m, 4H+6H).

### Example 30

### General Procedure (B)

### 6-Adamantan-1-yl-5-hydroxy-indan-4-carboxylic acid (2-chloro-4-trifluoromethanesulfonyl-phenyl)-amide

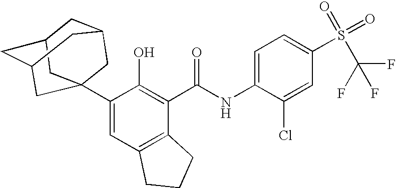

### Step A:

6-adamantan-1-yl-indan-5-ol From 5-indanol and adamantan-1-ol in H₂SO₄/CH₂Cl₂; beige solid, mp 169-171° C.; ¹H NMR (CDCl₃) δ 7.08 (s, 1H), 6.53 (s, 1H), 4.61 (br s, 1H), 2.85-2.78 (m, 4H), 2.14-2.09 (m, 6H), 2.09-2.00 (m, 5H), 1.79-1.73 (m, 6H).

### Step B:

From 6-adamantan-1-yl-indan-5-ol and 2-chloro-1-isocyanato-4-trifluoromethanesulfonyl-benzene, cream-colored crystals, mp 218-219° C.; HPLC-MS (Method B): m/z: 554 (M+H), 556 (M+2+H), 576 (M+Na), 578 (M+2+Na); Rₜ=3.46 min.; ¹H NMR (CDCl₃) δ 11.92 (s, 1H), 8.95 (d, 1H), 8.77 (br s, 1H), 8.09 (d, 1H), 7.99 (dd, J=8.59, 2.02 Hz, 1H), 7.34 (s, 1H), 3.36-3.28 (m, 2H), 2.95-2.88 (m, 2H), 2.25-2.12 (m, 2H+6H), 2.12-2.05 (br, 3H), 1.84-1.73 (m, 6H).

### Example 31

### General Procedure (B)

### 4-(4-Fluoro-phenylsulfanyl)-1-hydroxy-naphthalene-2-carboxylic acid (2-chloro-4-trifluoromethanesulfonyl-phenyl)-amide

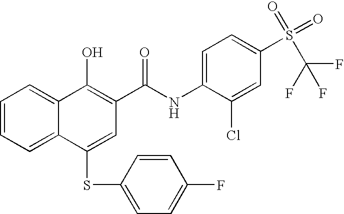

From 4-(4-fluoro-phenylsulfanyl)-naphthalen-1-ol and 2-chloro-1-isocyanato-4-trifluoromethanesulfonyl-benzene, pale yellow voluminous crystals, mp 195-198° C.; HPLC-MS (Method B): m/z: 556 (M+H), 558 (M+2+H), 578 (M+Na), 580 (+2+Na), Rₜ=2.93 min.; ¹H NMR (DMSO-d₆): δ 13.10 (s, 1H), 8.91 (d, J=9.10 Hz, 1H), 8.78 (br s, 1H), 8.53 (d, J=7.58 Hz, 1H), 8.27 (d, J=8.08 Hz, 1H), 8.11 (d, J=2.02 Hz, 1H), 8.00 (dd, J=2.02 Hz, 9.10 Hz, 1H), 7.74-7.68 (m, 1H), 7.66-7.60 (m+s at 7.62 ppm, 2H), 7.25-7.19 (m, 2H), 7.02-6.95 (m, 2H). The starting compound 4-(4-fluoro-phenylsulfanyl)-naphthalen-1-ol was prepared by sulfenylation of naphthalen-1-ol with 4-fluoro-phenylsulfenyl chloride by a procedure similar to the one reported in the U.S. Pat. No. 3,622,328.

### Example 32

### General Procedure (B)

### 5-Adamantan-1-yl-2′,4′-difluoro-4-hydroxy-biphenyl-3-carboxylic acid (2-chloro-4-trifluoromethanesulfonyl-phenyl)-amide

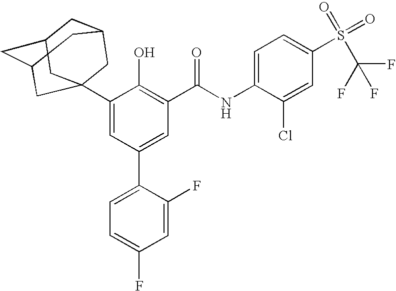

### Step A:

### 3-Adamantan-1-yl-2′,4′-difluoro-biphenyl-4-ol

From 2′,4′-difluoro-4-hydroxy-biphenyl and adamantan-1-ol in H₂SO₄/HOAc; pale crystals, mp 146-148° C.; TLC (toluene-heptane 7:3) R=0.37; ¹H NMR (CDCl₃) δ 7.40-7.31 (m, 2H), 7.20 (dt, J=8.08, 2.02 Hz, 1H), 6.94-6.84 (m, 2H), 4.86 (s, 1H), 2.18-2.13 (m, 6H), 2.12-2.06 (br, 3H), 1.81-1.75 (m, 6H).

### Step B:

From 3-adamantan-1-yl-2′,4′-difluoro-biphenyl-4-ol and 2-chloro-1-isocyanato-4-trifluoromethanesulfonyl-benzene, pale crystals, mp 177-178° C.; HPLC-MS (Method B): m/z: 626 (M+H), 628 (M+2+H), 648 (M+Na), 650 (M+2+Na); Rₜ=3.38 min.; ¹H NMR (CDCl₃) δ 12.12 (s, 1H), 8.92 (s, 1H), 8.88 (d, 1H), 8.11 (br (d), 1H), 8.01 (br (dd), 1H), 7.58 (s, 1H), 7.57 (s, 1H), 7.45-7.36 (m, 1H), 7.04-6.90 (m, 2H), 2.20 (br s, 6H), 2.12 (br s, 3H), 1.81 (br s, 6H).

### Example 33

### 4-(4-Fluoro-benzenesulfinyl)-1-hydroxy-naphthalene-2-carboxylic acid (2-chloro-4-trifluoromethanesulfonyl-phenyl)-amide

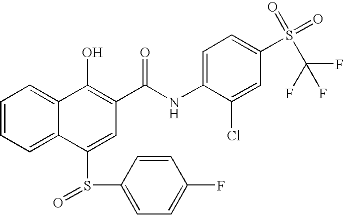

A solution of 77% m-chloroperbenzoic acid (0.036 g, 0.16 mmol) in dichloromethane (1 ml) was added at room temperature to a solution of 4-(4-fluorophenylsulfanyl)-1-hydroxy-naphthalene-2-carboxylic acid (2-chloro-4-trifluoromethanesulfonyl-phenyl)-amide. (0.081 g, 0.15 mmol) in dichloromethane (4 ml). The mixture was stirred overnight and the precipitate was collected by filtration, rinsed with dichloromethane and dried to give the title compound as a white solid. Yield 0.071 g (86%); mp 198-199.5° C.; HPLC-MS (Method B): m/z: 572 (M+H), 574 (M+2+H), 594 (M+Na), 596 (M+2+Na); Rₜ=2.48 min.; TLC (EtOAcMeOH 9:1): R_{f}=0.32; ¹H-NMR (DMSO-d₆): δ 13.6 (br, 1H), 9.03-8.95 (br m, 1H), 8.61-8.56 (br m, 1H), 8.44 (d, 1H), 8.26 (br s-like (d), 1H), 8.17-8.12 (br d-like, 1H), 8.11-8.05 (br dd, 1H), 7.75-7.67 (m, 2H), 7.60-7.44 (m, 2H), 7.37-7.29 (m, 2H).

### Example 34

### General Procedure (B)

### 5-tert-Butyl-2′,4′-difluoro-4-hydroxy-biphenyl-3-carboxylic acid (2-chloro-4-trifluoromethanesulfonyl-phenyl)-amide

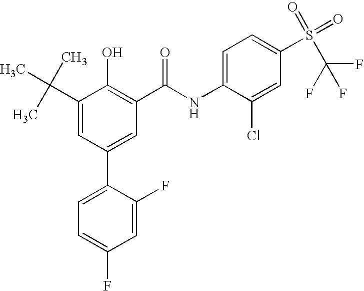

### Step A:

### 3-tert-butyl-2′,4′-difluoro-biphenyl-4-ol

From 2′,4′-difluoro-biphenyl-4-ol and t-butanol in H₂SO₄/HOAc; yellow oil; TLC (toluene-heptane 7:3) R_{f}=0.36; ¹H NMR (CDCl₃) δ 7.40-7.32 (m, 2H), 7.21 (dt, J=8.08, 2.02 Hz, 1H), 6.95-6.85 (m, 2H), 6.72 (d, J=8.08 Hz, 1H), 4.90 (s, 1H), 1.44 (s, 9H).

### Step B:

From 3-tert-butyl-2′,4′-difluoro-biphenyl-4-ol and 2-chloro-1-isocyanato-4-trifluoromethanesulfonyl-benzene; white crystals, mp 114-115° C.; HPLC-MS (Method B): m/z: 588 (M+H), 590 (M+2+H), 610 (M+Na), 612 (M+2+Na); Rₜ=3.22 min.; ¹H NMR (CDCl₃) δ 12.09 (s, 1H), 8.92 (s, 1H, NH), 8.89 (d, 1H), 8.11 (br (d), 1H), 8.01 (br (dd), 1H), 7.63 (s, 1H), 7.59 (s, 1H), 7.45-7.36 (m, 1H), 7.05-6.91 (m, 2H), 1.48 (s, 9H).

### Example 35

### General Procedure (B)

### 2′,4′-Difluoro-4-hydroxy-5-(1-methyl-cyclohexyl)-biphenyl-3-carboxylic acid (2-chloro-4-trifluoromethanesulfonyl-phenyl)-amide

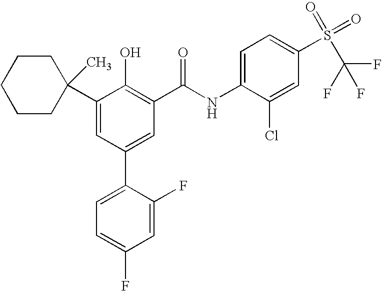

### Step A:

2′,4′-difluoro-3-(1-methyl-cyclohexyl)-biphenyl-4-ol From 2′,4′-difluoro-4-hydroxy-biphenyl and 1-methyl-cyclohexanol in CF₃SO₃H/toluene; pale crystals, mp 60-62° C.; ¹H NMR (CDCl₃) δ d 7.41 (br, 1H), 7.40-7.33 (m, 1H), 7.20 (dt, J=8.08, 2.02 Hz, 1H), 6.95-6.85 (m, 2H), 6.72 (d, J=8.08, 2.02 Hz, 1H), 2.23-2.14 (m, 2H), 1.76-1.67 (m, 6H), 1.36 (s, 3H, CH₃).

### Step B:

From 2′,4′-difluoro-3-(1-methyl-cyclohexyl)-biphenyl-4-ol and 2-chloro-1-isocyanato-4-trifluoromethanesulfonyl-benzene; white crystals, mp 114-115° C.; HPLC-MS (Method B): m/z: 588 (M+H), 590 (M+2+H), 610 (M+Na), 612 (M+2+Na); Rₜ=3.22 min.; ¹H NMR (CDCl₃) δ 12.12 (s, 1H), 8.92 (s, 1H), 8.89 (d, J=8.6 Hz, 1H, H), 8.11 (br (d), 1H), 8.01 (br d (dd), J=8.08 Hz 1H), 7.65 (br s, 1H), 7.59 (br s1H), 7.48-7.33 (m, 1H), 7.06-6.89 (m, 2H), 2.32-2.13 (br m, 2H), 1.88-1.71 (br m, 2H), 1.71-1.28 (br m+s, 9H; s at 1.41, CH₃).

### Example 36

### General Procedure (A)

### 4-(4-Ethanesulfonyl-phenyl)-1-hydroxy-naphthalene-2-carboxylic acid (2-chloro-4-trifluoromethanesulfonyl-phenyl)-amide

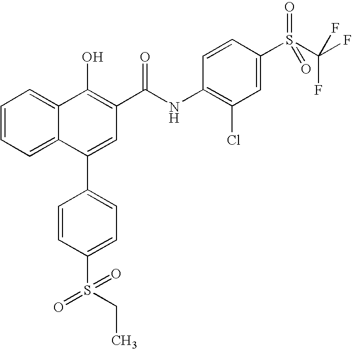

### Step A:

4-(4-Ethanesulfonyl-phenyl)-1-hydroxy-naphthalene-2-carboxylic acid from 4-bromo-1-hydroxy-naphthalene-2-carboxylic acid and 4-(ethanesulfonyl)-phenylboronic acid.

¹H NMR (DMSO-d₆): δ 8.43 (d, J=7.58 Hz, 1H) 8.03 (m, 4H) 7.77 (m, 4H) 3.4 (q hidden under D2O, 2H) 1.18 (t, J=7.33 Hz, 3H); HPLC-MS (Method A): m/z=357 (M+1); Rₜ=3.95 min

### Step B:

From the product formed in step A and 2-chloro-4-trifluoromethanesulfonyl aniline.

¹H NMR (DMSO-d₆): δ 8.73 (d, J=8.59 Hz, 1H) 8.51 (d, J=8.08 Hz, 1H) 8.33 (d, J=2.02 Hz, 1H) 8.19 (dd, J=8.59 and 2.02 Hz, 1H) 8.04 (m, 3H) 7.80 (m, 3H) 7.67 (m, 2H) 3.4 (q hidden under D20, 2H) 1.19 (t, J=7.58 Hz, 3H); HPLC-MS (Method A): m/z=598 (M⁺); Rₜ=5.31 min.

### Example 37

### General Procedure (A)

### 1-Hydroxy-4-(4-methoxy-phenyl)-naphthalene-2-carboxylic acid (2-chloro-4-trifluoromethanesulfonyl-phenyl)-amide

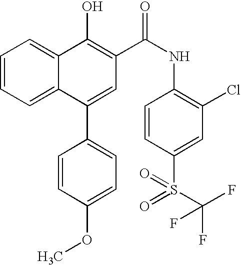

### Step A:

### 1-Hydroxy-4-(4-methoxy-phenyl)-naphthalene-2-carboxylic acid from 4-bromo-1-hydroxy-naphthalene-2-carboxylic acid and 4-methoxy-phenylboronic acid

¹H NMR (DMSO-d₆): δ 12.67 (br s, 1H) 8.39 (d, J=8.08 Hz, 1H) 7.79 (d, J=7.58 Hz, 1H) 7.65 (m, 2H) 7.61 (s, 1H) 7.38 (d, J=8.59 Hz, 2H) 7.08 (d, J=8.59 Hz, 2H) 3.84 (s, 3H); HPLC-MS (Method B): m/z=295 (M+1); Rₜ=1.82 min.

### Step B:

From the product formed in step A and 2-chloro-4-trifluoromethanesulfonyl aniline.

¹H NMR (DMSO-d₆): δ 11.67 (br s, 1H) 8.66 (d, 1H) 8.47 (d, 1H) 8.35 (s, 1H) 8.22 (d, 1H) 7.99 (s, 1H) 7.81 (d, 1H) 7.67 (m, 2H) 7.42 (d, 2H) 7.12 (d, 2H) 3.85 (s, 3H); HPLC-MS (Method B): m/z=537 (M+1); Rₜ=2.89 min.

### Example 38

### General Procedure (A)

### 4-(4-Fluoro-phenyl)-1-hydroxy-naphthalene-2-carboxylic acid (2-chloro-4-trifluoromethanesulfonyl-phenyl)-amide

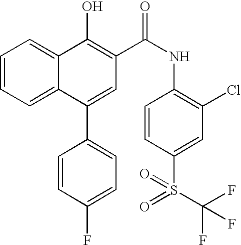

### Step A:

4-(4-Fluoro-phenyl)-1-hydroxy-naphthalene-2-carboxylic acid from 4-bromo-1-hydroxy-naphthalene-2-carboxylic acid and 4-fluoro-phenylboronic acid.

¹H NMR (DMSO-d₆): δ 12.71 (br s, 1H) 8.41 (d, J=7.58 Hz, 1H) 7.70 (m, 3H) 7.62 (s, 1H) 7.50 (dd, J=8.59 and 5.56 Hz, 2H) 7.35 (dd, J=9.1 Hz, 2H); HPLC-MS (Method B): m/z=283 (M+1); Rₜ=1.85 min.

### Step B:

From the product formed in step A and 2-chloro-4-trifluoromethanesulfonyl aniline.

¹H NMR (DMSO-d₆): δ 11.67 (br s, 1H) 8.65 (d, 1H) 8.48 (d, 1H) 8.36 (s, 1H) 8.22 (d, 1H) 8.02 (s, 1H) 7.76 (d, 1H) 7.68 (m, 2H) 7.54 (m, 2H) 7.39 (m, 2H); HPLC-MS (Method B): m/z=524 (M⁺); Rₜ=2.87 min.

### Example 39

### General Procedure (A)

### 1-Hydroxy-4-p-tolyl-naphthalene-2-carboxylic acid (2-chloro-4-trifluoromethanesulfonyl-phenyl)-amide

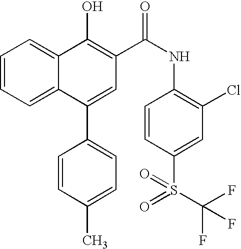

### Step A:

1-Hydroxy-4-p-tolyl-naphthalene-2-carboxylic acid from 4-bromo-1-hydroxynaphthalene-2-carboxylic acid and 4-methyl-phenylboronic acid.

¹H NMR (DMSO-d₆): δ 12.65 (br s, 1H) 8.39 (d, J=8.08 Hz, 1H) 7.78 (d, J=8.08 Hz, 1H) 7.65 (m, 2H) 7.61 (s, 1H) 7.33 (m, 4H) 2.40 (s, 3H); HPLC-MS (Method B): m/z=279 (M+1); Rₜ=1.96 min.

### Step B:

From the product formed in step A and 2-chloro-4-trifluoromethanesulfonyl aniline.

¹H NMR (DMSO-d₆): δ 11.69 (br s, 1H) 8.67 (d, 1H) 8.48 (d, 1H) 8.35 (s, 1H) 8.22 (d, 1H) 8.00 (s, 1H) 7.82 (d, 1H) 7.67 (m, 2H) 7.40 (d, 2H) 7.36 (d, 2H) 2.42 (s, 3H);

HPLC-MS (Method B): m/z=520 (M+1); Rₜ=3.02 min.

### Example 40

### General Procedure (A)

### 4-(4-Cyano-phenyl)-1-hydroxy-naphthalene-2-carboxylic acid (2-chloro-4-trifluoromethanesulfonyl-phenyl)-amide

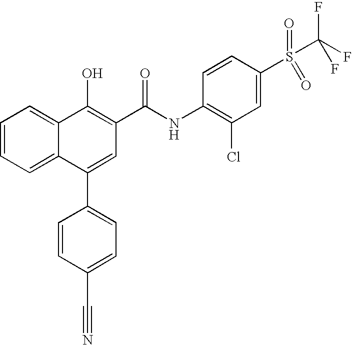

### Step A:

4-(4-Cyano-phenyl)-1-hydroxy-naphthalene-2-carboxylic acid from 4-bromo-1-hydroxy-naphthalene-2-carboxylic acid and 4-cyano-phenylboronic acid.

¹H NMR (DMSO-d₆): δ 12.85 (br s, 1H) 8.42 (d, J=7.58 Hz, 1H) 7.99 (d, J=8.08 Hz, 2H) 7.71 (m, 6H); HPLC-MS (Method B): m/z=290 (M+1); Rₜ=2.03 min.

### Step B:

From the product formed in step A and 2-chloro-4-trifluoromethanesulfonyl aniline.

HPLC-MS (Method A): m/z=531 (M+1) and 553 (M+23); Rₜ=5.64 min.

### Example 41

### General Procedure (A)

### 4-(3,5-Difluoro-phenyl)-1-hydroxy-naphthalene-2-carboxylic acid (2-chloro-4-trifluoromethanesulfonyl-phenyl)-amide

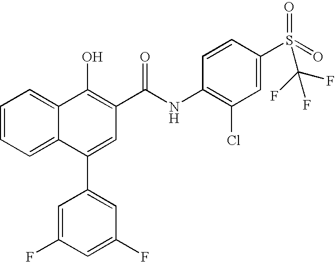

### Step A:

4-(3,5-Difluoro-phenyl)-1-hydroxy-naphthalene-2-carboxylic acid from 4-bromo-1-hydroxy-naphthalene-2-carboxylic acid and 3,5-difluoro-phenylboronic acid.

¹H NMR (DMSO-d₆): δ 8.41 (d, J=7.07 Hz, 1H) 7.78 (d, J=8.08 Hz, 1H) 7.70 (m, 3H) 7.34 (m, 1H) 7.22 (m, 2H); HPLC-MS (Method A): m/z=301 (M+1); Rₜ=4.48 min.

### Step B:

From the product formed in step A and 2-chloro-4-trifluoromethanesulfonyl aniline.

¹H NMR (DMSO-d₆): δ 11.88 (br s, 1H) 8.65 (d, J=8.59 Hz, 1H) 8.50 (d, J=8.08 Hz, 1H) 8.36 (d, J=2.02 Hz, 1H) 8.21 (dd, J=8.59 and 2.02 Hz, 1H) 8.05 (s, 1H) 7.82 (d, J=8.08 Hz, 1H) 7.69 (m, 2H) 7.37 (m, 1H) 7.27 (m, 2H); HPLC-MS (Method A): m/z=542 (M+1); Rₜ=5.97 min.

### Example 42

### General Procedure (A)

### 1-Hydroxy-4-(3-trifluoromethyl-phenyl)-naphthalene-2-carboxylic acid (2-chloro-4-trifluoromethanesulfonyl-phenyl)-amide

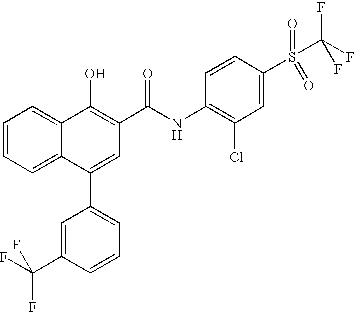

### Step A:

1-Hydroxy-4-(3-trifluoromethyl-phenyl)-naphthalene-2-carboxylic acid from 4-bromo-1-hydroxy-naphthalene-2-carboxylic acid and 3-trifluoromethyl-phenylboronic acid.

¹H NMR (DMSO-d₆): δ 12.8 (br s, 1H) 8.42 (d, J=7.58 Hz, 1H) 8.86-7.64 (m, 8H); HPLC-MS (Method B): m/z=333 (M+1); Rₜ=2.33 min.

### Step B:

From the product formed in step A and 2-chloro-4-trifluoromethanesulfonyl aniline.

¹H NMR (DMSO-d₆): δ 11.69 (br s, 1H) 8.63 (d, 1H) 8.52 (d, 1H) 8.36 (s, 1H) 8.22 (d, 1H) 8.06 (s, 1H) 7.83 (m, 4H) 7.69 (m, 3H); HPLC-MS (Method B): m/z=574 (M+1); Rₜ=2.99 min.

### Example 43

### General Procedure (A)

### 4-(4-Acetyl-phenyl)-1-hydroxy-naphthalene-2-carboxylic acid (2-chloro-4-trifluoromethanesulfonyl-phenyl)-amide

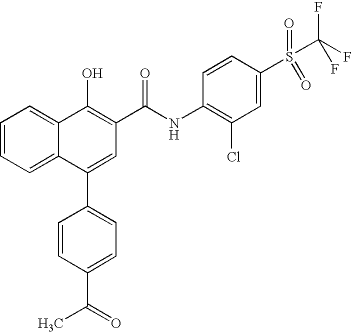

### Step A:

4-(4-Acetyl-phenyl)-1-hydroxy-naphthalene-2-carboxylic acid from 4-bromo-1-hydroxy-naphthalene-2-carboxylic acid and 4-acetyl-phenylboronic acid.

¹H NMR (DMSO-d₆): δ 12.8 (br s, 1H) 8.42 (d, J=7.58 Hz, 1H) 8.11 (d, J=8.59 Hz, 2H) 7.78 (d, J=8.08 Hz, 1H) 7.69 (m, 3H) 7.63 (d, J=8.59 Hz, 2H) 2.66 (s, 3H); HPLC-MS (Method B): m/z=307 (M+1); Rₜ=1.997 min.

### Step B:

From the product formed in step A and 2-chloro-4-trifluoromethanesulfonyl aniline.

¹H NMR (DMSO-d₆): δ 11.83 (br s, 1H) 8.68 (d, J=8.59 Hz, 1H) 8.51 (m, 1H) 8.35 (d, J=2.02 Hz, 1H) 8.21 (dd, J=8.59 and 2.02 Hz, 1H) 8.14 (d, J=8.59 Hz, 2H) 8.06 (s, 1H) 7.81 (m, 1H) 7.68 (m, 4H) 2.67 (s, 3H); HPLC-MS (Method B): m/z=548 (M+1); Rₜ=2.76 min. Prep. HPLC-MS (Method C): m/z=548 (M+1); Rₜ=9.10 min.

### Example 44

### General Procedure (B)

### 2-Hydroxy-5-methyl-3-(1,1,2,2-tetramethyl-propyl)-N-(4-trifluoromethanesulfonylphenyl)-benzamide

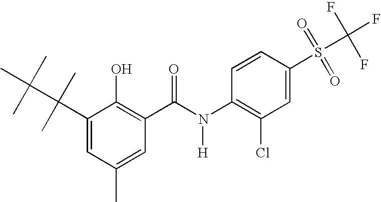

### Step A:

### 4-Methyl-2-(1,1,2,2-tetramethyl-propyl)-phenol

From 4-methyl-phenol in H₂SO₄/CH₂Cl₂; colourless oil from column chromatography on silica with toluene-heptane 7:3 as eluent. TLC (toluene-heptane 7:3) R_{f}=0.4; ¹H NMR (CDCl₃) δ 7.06 (d, J=2.02 Hz, 1H), 6.86 (dd, J=2.02, 8.08 Hz, 1H), 6.55 (d, J=8.08 Hz), 4.62 (s, 1H, OH), 2.26 (s, 3H, 1.46 (s, 6H), 0.91 (s, 9H).

### Step B:

From 4-methyl-2-(1,1,2,2-tetramethyl-propyl)-phenol and 2-chloro-1-isocyanato-4-trifluoromethanesulfonyl-benzene; ¹H NMR (CDCl₃) δ 11.79 (s, 1H, OH), 8.88 (s, 1H, NH), 8.87 (d, J=9.10 Hz, 1H), 8.11 (d, J=2.02 Hz, 1H), 7.99 (dd, J=2.02 Hz, 9.10 Hz, 1H), 7.38 (s, 1H), 7.22 (s, 1H), 2.36 (s, 3H), 1.49 (s, 6H), 0.92 (s, 9H); HPLC-MS (Method B): m/z: 492 (M+H), 494 (M+2+H); Rₜ=3.09 min.

### Example 45

### General Procedure (B)

### 4′-Bromo-5-tert-butyl-4-hydroxy-biphenyl-3-carboxylic acid (2-chloro-4-trifluoromethanesulfonyl-phenyl)-amide

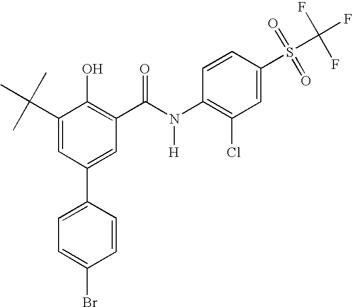

### Step A:

### 4′-Bromo-3-tert-butyl-biphenyl-4-ol

From 4′-bromo-biphenyl-4-ol in H₂SO₄/HOAc; white crystals, from column chromatography on silica with toluene-heptane 7:3 as eluent. TLC (toluene-heptane 7:3) R_{f}=0.29;

¹H NMR (CDCl₃) δ 7.52 (2H) and 7.40 (2H) (J_{AB}-system, J=8.59 Hz), 7.45 (d, J=2.02 Hz 1H), 7.25 (dd, 1H), 6.73 (d, J=8.08 Hz, 1H), 1.45 (s, 9H).

### Step B:

From 4′-bromo-3-tert-butyl-biphenyl-4-ol and 2-chloro-1-isocyanato-4-trifluoromethanesulfonyl-benzene; ¹H NMR (CDCl₃) δ 12.07 (s, 1H, OH), 8.93 (br s, 1H, NH), 8.89 (d, J=8.59 Hz, 1H), 8.12 (d, J=2.02 Hz, 1H), 8.02 (dd, 1H), 7.70 (d, J=1.52 Hz, 1H), 7.54 (d, J=1.52 Hz, 1H), 7.60 (2H) and 7.40 (2H) (J_{AB}-system), 1.49 (s, 9H); HPLC-MS (Method B): m/z: 590 (M+H), 592 (M+2+H); Rₜ=3.13 min.

### Example 46

### General Procedure (B)

### 3-tert-Butyl-N-(2-chloro-4-trifluoromethanesulfonyl-phenyl)-6-ethyl-2-hydroxybenzamide

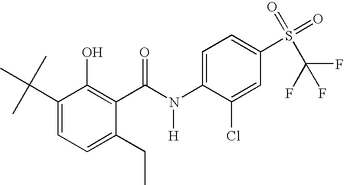

From 2-tert-butyl-5-ethyl-phenol and 2-chloro-1-isocyanato-4-trifluoromethanesulfonyl-benzene; voluminous white crystals, mp 126-127° C.; ¹H NMR (CDCl₃): δ 9.30 (s, 1H), 9.09 (d, J=9.09 Hz, H6′), 8.45 (br s, 1H), 8.09 (d, J=2.02 Hz, 1H), 8.01 (dd, 1H), 7.37 (d, J=8.08 Hz, 1H), 6.84 (d, J=8.08 Hz, 1H), 2.91 (q, J=7.58 Hz, 2H), 1.42 (s, 9H), 1.32 (t, 3H) ppm; HPLC-MS (Method B): m/z: 464 (M+H), 466 (M+2+H), 486 (M+Na), 488 (M+2+Na), Rₜ=2.68 min.

### Example 47

### General Procedure (B)

### 3,5-Di-tert-butyl-N-(2-chloro-4-trifluoromethanesulfonyl-phenyl)-2-ethyl-6-hydroxybenzamide

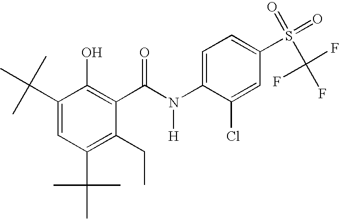

From 2,4-Di-tert-butyl-5-ethyl-phenol and 2-chloro-1-isocyanato-4-trifluoromethanesulfonyl-benzene; white crystals; mp 157-158° C.; ¹H NMR (CDCl₃) δ 9.01 (br d-like, 1H), 8.41 (br s, 1H), 8.09 (d, J=2.0 Hz, 1H), 8.01 (br dd, 1H), 7.47 (s, 1H), 7.12 (s, 1H), 3.13 (q, J=7.58 Hz, 2H), 1.44 (s, 9H), 1.42 (s, 9H), 1.16 (t, 3H) ppm; HPLC-MS (Method B): m/z: 520 (M+H), 522 (M+2+H), 542 (M+Na), 544 (M+2+Na), 558 (M+K), 560 (M+2+K), Rₜ=2.86 min.

### Example 48

### General Procedure (C)

### 6-tert-Butyl-5-hydroxy-indan-4-carboxylic acid (2-chloro-4-trifluoromethanesulfonylphenyl)-amide

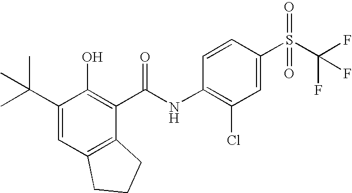

From 6-tert-Butyl-indan-5-ol and 2-chloro-1-isocyanato-4-trifluoromethanesulfonyl-benzene; pale yellow crystals, mp 172-173° C.; ¹H NMR (CDCl₃) δ 11.91 (s, 1H), 8.96 (d, J=8.59 Hz, 1H), 8.78 (s, 1H), 8.10 (d, J=2.02 Hz, 1H), 8.00 (dd, 1H), 7.40 (s, 1H), 3.33 (t, 2H), 2.92 (t, 2H), 2.21 (quintet, 2H), 1.43 (s, 9H) ppm; HPLC-MS (Method B): m/z: 476 (M+H), 478 (M+2+H), 498 (M+Na), 500 (M+2+Na), Rₜ=2.86 min.

### Example 49

### General Procedure (C)

### 3-tert-Butyl-N-(2-chloro-4-trifluoromethanesulfonyl-phenyl)-2-hydroxy-6-methyl-5-methylsulfanyl-benzamide

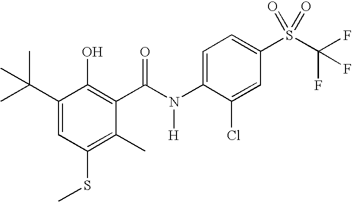

From 2-tert-butyl-5-methyl-4-methylsulfanyl-phenol and 2-chloro-1-isocyanato-4-trifluoromethanesulfonyl-benzene; white crystals, mp 155.5-156.5° C.; ¹H NMR (CDCl₃) δ 9.39 (s, 1H), 9.01 (d, J=9.09 Hz, 1H), 8.45 (br s, 1H), 8.09 (d, J=1.52 Hz, 1H), 8.02 (dd, 1H), 7.46 (s, 1H), 2.67 (s, 3H), 2.45 (s, 1H), 1.42 (s, 9H); HPLC-MS (Method B): m/z: 496 (M+H), 498 (M+2+H), 518 (M+Na), 520 (M+2+Na), Rₜ=2.76 min.

### Example 50

### General Procedure (C)

### 3-tert-Butyl-2-hydroxy-5,6,7,8-tetrahydro-naphthalene-1-carboxylic acid (2-chloro-4-trifluoromethanesulfonyl-phenyl)-amide

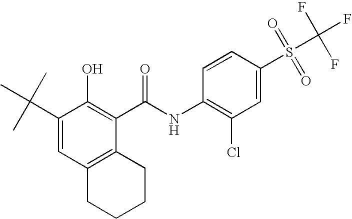

From 3-tert-butyl-5,6,7,8-tetrahydro-naphthalen-2-ol and 2-chloro-1-isocyanato-4-trifluoromethanesulfonyl-benzene; white crystals, mp 151° C. (from acetonitrile); ¹H NMR (CDCl₃) δ 9.45 (s, 1H), 9.01 (d, 1H), 8.49 (s, 1H), 8.08 (d, J=1.52 Hz, 1H), 8.00 (dd, 1H), 7.15 (s, 1H), 2.96-2.89 (m, 2H), 2.82-2.74 (m, 2H), 1.89-1.72 (m, 4H), 1.41 (s, 9H) ppm; HPLC-MS (Method B): m/z: 490 (M+H), 492 (M+2+H), Rₜ=2.92 min.

### Example 51

### 3-tert-Butyl-N-(2-chloro-4-trifluoromethanesulfonyl-phenyl)-2-hydroxy-5-methanesulfinyl-6-methyl-benzamide

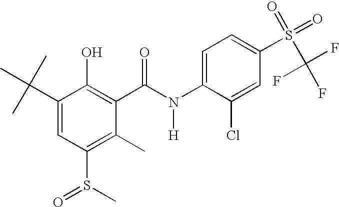

A solution of m-CPBA (77% purity, 0.050 g, 0.22 mmol) in dichloromethane (3 ml) was added dropwise at room temperature to a stirred solution of 3-tert-butyl-N-(2-chloro-4-trifluoromethanesulfonyl-phenyl)-2-hydroxy-6-methyl-5-methylsulfanyl-benzamide (0.10 g, 0.2 mmol) in dichloromethane (3 ml) and the mixture was stirred overnight. The solvent was removed in vacuo and the residue was extracted with ether. The insoluble residue was collected by filtration and dried to give the title compound, white crystals, mp 218-219° C. (decomp.); ¹H NMR (DMSO-d₆) δ 10.72 (br s, 1H), 9.60 (s, 1H), 8.71-8.56 (m, 1H), 8.30-8.14 (m, 2H), 7.76 (s, 1H), 2.66 (s, 3H), 2.27 (s, 3H), 1.42 (s, 9H) ppm; HPLC-MS (Method B): m/z: 512 (M+H), 514 (M+2+H), 534 (M+Na), 536 (M+2+Na) Rₜ=2.14 min.

### Example 52

### 3-tert-Butyl-N-(2-chloro-4-trifluoromethanesulfonyl-phenyl)-2-hydroxy-5-methanesulfonyl-6-methyl-benzamide

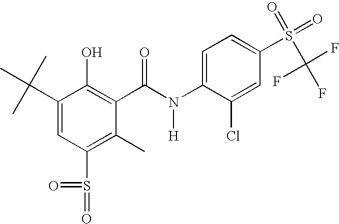

From 3-tert-butyl-N-(2-chloro-4-trifluoromethanesulfonyl-phenyl)-2-hydroxy-6-methyl-5-methylsulfanyl-benzamide and two equivalents of m-CPBA in dichloromethane; pale crystals, mp 220-221° C.; ¹H NMR (DMSO-d₆) δ 10.89 (br s, 1H), 10.11 (s, 1H), 8.72-8.62 (m, 1H), 8.29-8.16 (m, 2H), 7.87 (s, 1H), 3.19 (s, 3H), 2.56 (s, 3H), 1.40 (s, 9H) ppm; HPLC-MS (Method B): m/z 528 (M+H), 530 (M+2+H), 550 (M+Na), 552 (M+2+Na), Rₜ=2.27 min.

### Example 53

### General Procedure (C)

### 3-tert-Butyl-N-(2-chloro-4-trifluoromethanesulfonyl-phenyl)-2-hydroxy-5-methoxy-benzamide

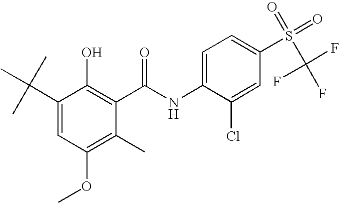

From 2-tert-butyl-4-methoxy-phenol and 2-chloro-1-isocyanato-4-trifluoromethanesulfonyl-benzene; pale yellow voluminous crystals, mp 126-127° C. (phase transition), and 137-138° C.; ¹H NMR (CDCl₃) δ 11.56 (s, 1H), 8.88 (d, J=9.10 Hz, 1H), 8.81 (br s, 1H), 8.11 (d, J=2.02 Hz 1H), 8.00 (dd, 1H), 7.20 (d, J=3.03 Hz, 1H), 6.85 (d, J=3.03 Hz, 1H), 3.84 (s, 3H), 1.44 (s, 9H); HPLC-MS (Method B): m/z: 466 (M+H), 468 (M+2+H), Rₜ=2.81 min.

### Example 54

### General Procedure (C)

### 3-tert-Butyl-N-(2-chloro-4-trifluoromethanesulfonyl-phenyl)-2-hydroxy-6-isopropyl-benzamide

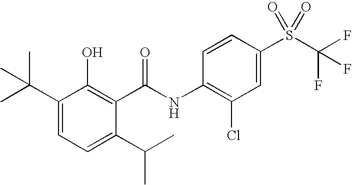

From 2-tert-butyl-5-isopropyl-phenol and 2-chloro-1-isocyanato-4-trifluoromethanesulfonyl-benzene; off-white crystals, mp 116-116.5° C.; ¹H NMR (CDCl₃): δ 9.02 (d, J=8.59 Hz, H6′), 8.42 (s, 1H), 8.30 (br s, 1H), 8.09 (d, J=2.02 Hz, 1H), 8.02 (dd, 1H), 7.41 (d, J=8.08 Hz, 1H), 6.93 (d, J=8.59 Hz, 1H), 3.26 (m, 1H), 1.41 (s, 9H), 1.33 (d, 6H) ppm; HPLC-MS (Method B): m/z: 478 (M+H), 480 (M+2+H), 500 (M+Na), 502 (M+2+Na), Rₜ=2.76 min.

### Example 55

### General Procedure (D)

### 5-tert-Butyl-3′,5′-difluoro-4-hydroxy-2-methyl-biphenyl-3-carboxylic acid (4-trifluoromethanesulfonyl-phenyl)-amide

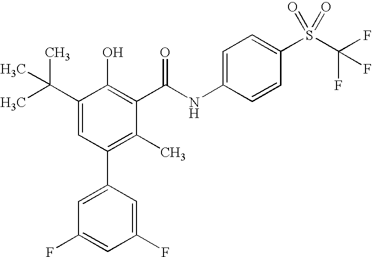

Step A: From 3-bromo-5-tert-butyl-6-hydroxy-2-methyl-benzoic acid and 3,5-difluorophenylboronic acid.

Step B: From the product formed in step A and 4-(trifluoromethylsulfonyl)aniline; ¹H NMR (DMSO-d₆): δ 1.39 (s, 9H) 2.11 (s, 3H) 7.00-7.06 (m, 2H) 7.11 (s, 1H) 7.23 (tt, J=9.42, 2.26 Hz, 1H) 8.11 (d, J=9.04 Hz, 2H) 8.18 (d, J=9.04 Hz, 2H) 9.10 (br. s., 1H) 11.23 (br. s., 1H) ppm; HPLC-MS (Method B): m/z: 528 (M+H), Rₜ=2.78 min.

### Example 56

### General Procedure (D)

### 5-tert-Butyl-4′-fluoro-4-hydroxy-2-methyl-biphenyl-3-carboxylic acid (4-trifluoromethanesulfonyl-phenyl)-amide

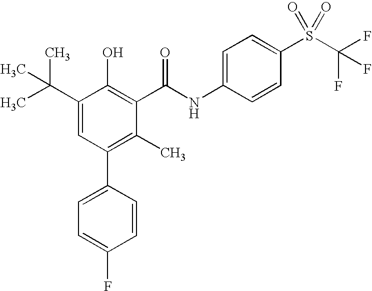

Step A: From 3-bromo-5-tert-butyl-6-hydroxy-2-methyl-benzoic acid and 4-fluorophenylboronic acid.

Step B: From the product formed in step A and 4-(trifluoromethylsulfonyl)aniline; ¹H NMR (DMSO-d₆): δ 1.39 (s, 9H) 2.08 (s, 3H) 7.08 (s, 1H) 7.22-7.37 (m, 4H) 8.11 (d, J=8.59 Hz, 2H) 8.19 (d, J=8.59 Hz, 2H) 8.96 (s, 1H) 11.22 (s, 1H) ppm; HPLC-MS (Method B): m/z: 510 (M+H), Rₜ=2.71 min.

### Example 57

### General Procedure (D)

### 5-tert-Butyl-3′,5′-difluoro-4-hydroxy-2-methyl-biphenyl-3-carboxylic acid (3-chloro-4-trifluoromethanesulfonyl-phenyl)-amide

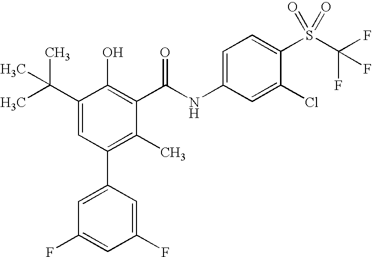

Step A: From 3-bromo-5-tert-butyl-6-hydroxy-2-methyl-benzoic acid and 3,5-difluorophenylboronic acid.

Step B: From the product formed in step A and 3-chloro-4-(trifluoromethylsulfonyl)aniline; ¹H NMR (DMSO-d₆): δ 1.39 (s, 9H) 2.10 (s, 3H) 6.98-7.07 (m, 2H) 7.12 (s, 1H) 7.23 (ft, J=9.42, 2.26 Hz, 1H) 8.01 (dd, J=9.04, 1.88 Hz, 1H) 8.24 (d, J=9.04 Hz, 1H) 8.33 (d, J=1.88 Hz, 1H) 9.14 (br. s., 1H) 11.37 (br. s., 1H) ppm; HPLC-MS (Method B): m/z: 562 (M+H), Rₜ=2.8 min.

### Example 58

### General Procedure (D)

### 5-tert-Butyl-4′-fluoro-4-hydroxy-2-methyl-biphenyl-3-carboxylic acid (3-chloro-4-trifluoromethanesulfonyl-phenyl)-amide

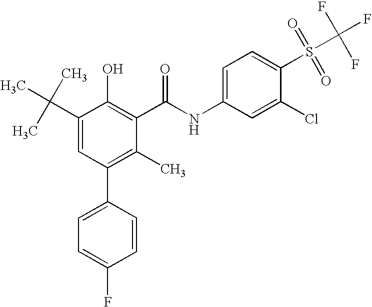

Step A: From 3-bromo-5-tert-butyl-6-hydroxy-2-methyl-benzoic acid and 4-fluorophenylboronic acid.

Step B: From the product formed in step A and 3-chloro-4-(trifluoromethylsulfonyl)aniline; ¹H NMR (DMSO-d₆): δ 1.39 (s, 9H) 2.07 (s, 3H) 7.09 (s, 1H) 7.22-7.36 (m, 4H) 8.01 (dd, J=9.10, 2.02 Hz, 1H) 8.24 (d, J=9.09 Hz, 1H) 8.33 (d, J=2.02 Hz, 1H) 9.01 (br. s., 1H) 11.36 (br. s., 1H) ppm; HPLC-MS (Method B): m/z: 544 (M+H), Rₜ=2.81 min.

### Example 59

### General Procedure (C)

### 3-tert-Butyl-N-(2-chloro-4-trifluoromethanesulfonyl-phenyl)-5-fluoro-2-hydroxy-6-methyl-benzamide

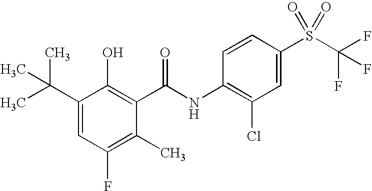

From 2-tert-butyl-4-fluoro-5-methylphenol and 2-chloro-1-isocyanato-4-trifluoromethanesulfonyl-benzene; ¹H NMR (CDCl₃): δ 1.41 (s, 9H) 2.53 (d, J=2.53 Hz, 3H) 7.18 (d, J=11.62 Hz, 1H) 8.02 (dd, J=9.10, 2.02 Hz, 1H) 8.10 (d, J=2.02 Hz, 1H) 8.48 (br. s., 1H) 9.00 (d, J=9.10 Hz, 1H) 9.79 (br. s., 1H) ppm; HPLC-MS (Method B): m/z: 468 (M+H), Rₜ=2.64 min.

### Example 60

### General Procedure (A)

### 3-tert-Butyl-5-chloro-N-(3-chloro-4-trifluoromethanesulfonyl-phenyl)-2-hydroxy-6-methyl-benzamide

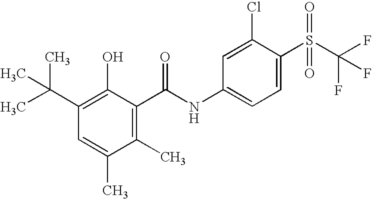

Step B: From 3-chloro-5-tert-butyl-6-hydroxy-2-methyl-benzoic acid and 3-chloro-4-trifluoromethanesulfonyl aniline.

¹H NMR (DMSO-d₆): δ 1.36 (s, 9H) 2.20 (s, 3H) 7.25 (s, 1H) 7.98 (dd, J=8.85, 1.70 Hz, 1H) 8.25 (d, J=9.04 Hz, 1H) 8.30 (d, J=1.88 Hz, 1H) 9.19 (s, 1H) 11.33 (s, 1H); HPLC-MS (Method B): m/z=484, 486 (M+1); Rₜ=2.64 min.

### Example 61

### General Procedure (A)

### 3-tert-Butyl-N-(2-chloro-4-trifluoromethanesulfonyl-phenyl)-2-hydroxy-5-iodo-6-methyl-benzamide

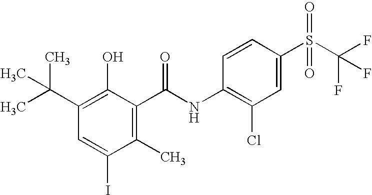

Step B: From 3-Iodo-5-tert-butyl-6-hydroxy-2-methyl-benzoic acid and 2-chloro-4-trifluoromethanesulfonyl aniline.

¹H NMR (DMSO-d₆): δ ppm 1.34 (s, 9H) 2.32 (s, 3H) 7.62 (s, 1H) 8.20 (d, J=8.67 Hz, 1H) 8.24 (s, 1H) 8.61 (d, J=8.67 Hz, 1H) 9.19 (s, 1H) 10.67 (s, 1H); HPLC-MS (Method B): m/z=576, 578 (M+1); Rₜ=2.82 min.

### Example 62

### General Procedure (A)

### 3-tert-Butyl-5-chloro-2-hydroxy-6-methyl-N-(4-trifluoromethanesulfonyl-phenyl)benzamide

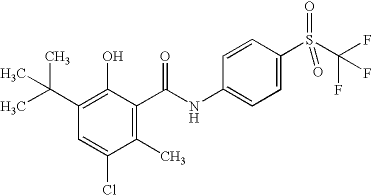

Step B: From 3-chloro-5-tert-butyl-6-hydroxy-2-methyl-benzoic acid and 4-trifluoromethanesulfonyl aniline.

¹H NMR (DMSO-d₆): δ ppm 1.36 (s, 9H) 7.24 (s, 1H) 8.05-8.23 (m, 4H) 9.14 (s, 1H) 11.19 (s, 1H); HPLC-MS (Method B): m/z=450, 452 (M+1); Rₜ=2.55 min.

### Example 63

### General Procedure (A)

### 5-tert-Butyl-3′,4′-difluoro-4-hydroxy-2-methyl-biphenyl-3-carboxylic acid (4-trifluoromethanesulfonyl-phenyl)-amide

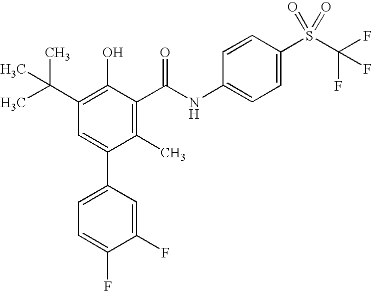

Step A: From 3-bromo-5-tert-butyl-6-hydroxy-2-methyl-benzoic acid and 3,4-difluoro phenyl-boronic acid.

Step B: From the product formed in step A and 4-trifluoromethanesulfonyl aniline.

HPLC-MS (Method B): m/z=528 (M+1); Rₜ=2.72 min.

### Example 64

### General Procedure (A)

### 5-tert-Butyl-3′,4′-difluoro-4-hydroxy-2-methyl-biphenyl-3-carboxylic acid (3-chloro-4-trifluoromethanesulfonyl-phenyl)-amide

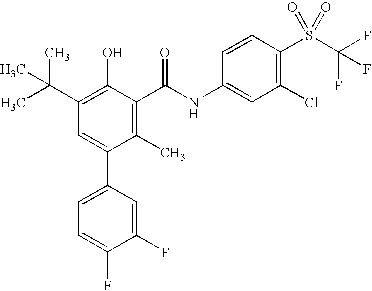

Step A: From 3-bromo-5-tert-butyl-6-hydroxy-2-methyl-benzoic acid and 3,4-difluoro phenyl-boronic acid.

Step B: From the product formed in step A and 3-chloro-4-trifluoromethanesulfonyl aniline

¹H NMR (DMSO-d₆): δ ppm 1.39 (s, 9H) 2.09 (s, 3H) 7.02-7.19 (m, 2H) 7.30-7.58 (m, 2H) 8.02 (d, 1H) 8.23 (d, J=8.67 Hz, 1H) 8.32 (s, 1H) 9.08 (s, 1H) 11.37 (s, 1H);

HPLC-MS (Method B): m/z=562, 564 (M+1); Rₜ=2.80 min.

### Example 65

### General Procedure (A)

### 5-tert-Butyl-4-hydroxy-4′-methoxy-2-methyl-biphenyl-3-carboxylic acid (2-chloro-4-trifluoromethanesulfonyl-phenyl)-amide

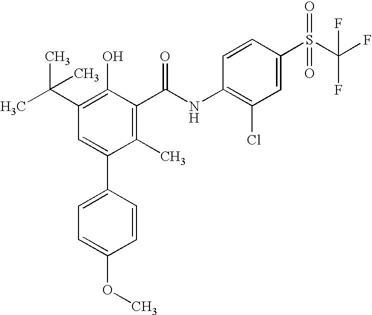

Step A: From 3-bromo-5-tert-butyl-6-hydroxy-2-methyl-benzoic acid and 4-methoxy phenyl-boronic acid.

Step B: From the product formed in step A and 2-chloro-4-trifluoromethanesulfonyl aniline

¹H NMR (DMSO-d₆): δ ppm 1.39 (s, 9H) 2.16 (s, 3H) 3.79 (s, 3H) 6.99 (d, J=8.67 Hz, 2H) 7.07 (s, 1H) 7.22 (d, J=8.67 Hz, 2H) 8.19 (d, J=8.67 Hz, 1H) 8.24 (s, 1H) 8.62 (d, J=8.67 Hz, 1H) 8.92 (s, 1H) 10.59 (s, 1H); HPLC-MS (Method B): m/z=556, 558 (M+1); Rₜ=2.94 min.

### Example 66

### General Procedure (A)

### 3-tert-Butyl-N-(2-chloro-4-trifluoromethanesulfonyl-phenyl)-5-(5-cyano-thiophen-2-yl)-2-hydroxy-6-methyl-benzamide

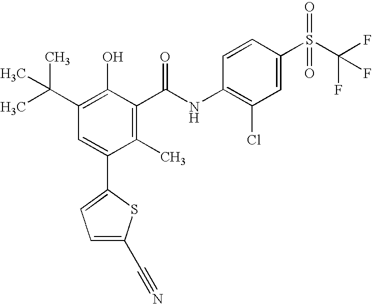

Step A: From 3-iodo-5-tert-butyl-6-hydroxy-2-methyl-benzoic acid and 5-cyano-2-thienyl-boronic acid.

Step B: From the product formed in step A and 2-chloro-4-trifluoromethanesulfonyl aniline

¹H NMR (CDCl₃): δ ppm 1.43 (s, 9H) 2.56 (s, 3H) 7.02 (dd, J=3.77, 1.88 Hz, 1H) 7.26 (d, J=1.88 Hz, 1H) 7.40 (s, 1H) 7.63 (dd, J=3.77, 1.88 Hz, 1H) 8.04 (d, J=9.04 Hz, 1H) 8.11 (s, 1H) 8.50 (s, 1H) 9.02 (d, J=9.04 Hz, 1H) 9.70 (s, 1H); HPLC-MS (Method B): m/z=557, 559 (M+1); Rₜ=2.76 min.

### Example 67

### General Procedure (A)

### 5-tert-Butyl-4′-dimethylsulfamoyl-4-hydroxy-2-methyl-biphenyl-3-carboxylic acid (2-chloro-4-trifluoromethanesulfonyl-phenyl)-amide

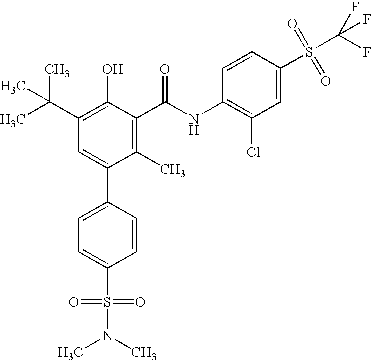

Step A: From 3-bromo-5-tert-butyl-6-hydroxy-2-methyl-benzoic acid and 4-dimethylsulfamoyl-boronic acid (SYNLETT (5): 892-894 APR 3 2004).

Step B: From the product formed in step A and 2-chloro-4-trifluoromethanesulfonyl aniline.

¹H NMR (CDCl₃): δ 1.45 (s, 9H) 2.44 (s, 3H) 2.80 (s, 6H) 7.29 (s, 1H) 7.48 (d, J=8.08 Hz, 2H) 7.86 (d, J=8.08 Hz, 2H) 8.03 (dd, J=8.59, 2.02 Hz, 1H) 8.11 (d, J=2.02 Hz, 1H) 8.54 (s, 1H) 9.04 (d, J=8.59 Hz, 1H) 9.60 (s, 1H); HPLC-MS (Method B): m/z=633, 635 (M+1); Rₜ=2.73 min.

### Example 68

### General Procedure (A)

### 5-tert-Butyl-4-hydroxy-2-methyl-4′-(morpholine-4-sulfonyl)-biphenyl-3-carboxylic acid (2-chloro-4-trifluoromethanesulfonyl-phenyl)-amide

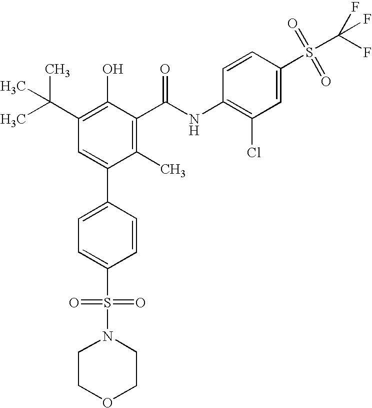

Step A: From 3-bromo-5-tert-butyl-6-hydroxy-2-methyl-benzoic acid and 4-morpholine-4-sulfonyl)phenyl-boronic acid (SYNLETT (5): 892-894 APR 3 2004).

Step B: From the product formed in step A and 2-chloro-4-trifluoromethanesulfonyl aniline.

¹H NMR (DMSO-d₆): δ ppm 1.39 (s, 9H) 2.16 (s, 3H) 3.79 (s, 3H) 6.99 (d, J=8.67 Hz, 2H) 7.07 (s, 1H) 7.22 (d, J=8.67 Hz, 2H) 8.19 (d, J=8.67 Hz, 1H) 8.24 (s, 1H) 8.62 (d, J=8.67 Hz, 1H) 8.92 (s, 1H) 10.59 (s, 1H); HPLC-MS (Method B): m/z=556, 558 (M+1); Rₜ=2.94

### Example 69

### General Procedure (A)

### 3-1,3-Benzodioxol-5-yl-5-tert-butyl-N-(2-chloro-4-trifluoromethanesulfonyl-phenyl)-6-hydroxy-2-methyl-benzamide

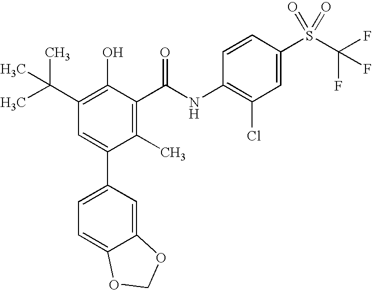

Step A: From 3-bromo-5-tert-butyl-6-hydroxy-2-methyl-benzoic acid and (3,4-methylenedioxyphenyl)boronic acid.

Step B: From the product formed in step A and 2-chloro-4-trifluoromethanesulfonyl aniline.

¹H NMR (DMSO-d₆): δ ppm 1.39 (s, 9H) 2.16 (s, 3H) 6.05 (s, 2H) 6.74 (dd, J=7.91, 1.51 Hz, 1H) 6.85 (d, J=1.51 Hz, 1H) 6.97 (d, J=7.91 Hz, 1H) 7.07 (s, 1H) 8.19 (d, J=7.91 Hz, 1H) 8.24 (s, 1H) 8.61 (d, 1H) 8.95 (s, 1H) 10.59 (s, 1H); HPLC-MS (Method B): m/z=570, 572 (M+1); Rₜ=2.74.

### Example 70

### General Procedure (A)

### 5-tert-Butyl-4-hydroxy-3′-methoxy-2-methyl-biphenyl-3-carboxylic acid (2-chloro-4-trifluoromethanesulfonyl-phenyl)-amide

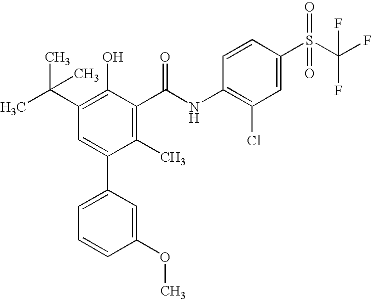

Step A: From 3-bromo-5-tert-butyl-6-hydroxy-2-methyl-benzoic acid and 3-methoxyphenyl-boronic acid.

Step B: From the product formed in step A and 2-chloro-4-trifluoromethanesulfonyl aniline.

¹H NMR (DMSO-d₆): δ ppm 1.40 (s, 9H), 2.17 (s, 3H) 3.77 (s, 3H) 6.82 (s, 1H) 6.87 (d, J=7.54 Hz, 1H) 6.92 (dd, J=7.91, 2.26 Hz, 1H) 7.10 (s, 1H) 7.35 (dd, 1H) 8.19 (dd, J=8.67, 2.26 Hz, 1H) 8.24 (d, J=2.26 Hz, 1H) 8.62 (d, J=8.67 Hz, 1H) 8.98 (s, 1H) 10.58 (s, 1H); HPLC-MS (Method B): m/z=556, 578 (M+1); Rₜ=2.96.

### Example 71

### General Procedure (A)

### 4-(3,4-Difluorophenyl)-1-hydroxynaphthalene-2-carboxylic acid (2-chloro-4-trifluoromethanesulfonylphenyl)amide

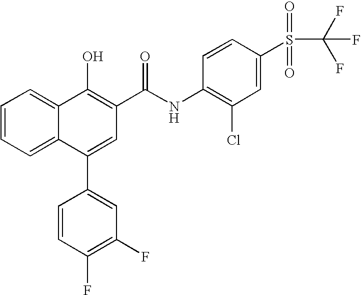

Step A: From 4-bromo-1-hydroxynaphthalene-2-carboxylic acid and 3,4-difluorophenylboronic acid.

Step B: From the product formed in step A and 2-chloro-4-trifluoromethanesulfonyl aniline

¹H NMR (DMSO-d₆): δ 7.35 (m, 1H) 7.57-7.72 (m, 4H) 7.79 (dd, 1H) 8.03 (s, 1H) 8.20 (dd, 1H) 8.35 (d, 1H) 8.47 (dd, 1H) 8.65 (d, 1H) 11.7 (s, 1H); HPLC-MS (Method B): m/z=542 (M+1); Rₜ=2.93 min.

### Example 72

### General Procedure (A)

### 1-Hydroxy-4-(4-trifluoromethylphenyl)naphthalene-2-carboxylic acid (2-chloro-4-trifluoromethanesulfonylphenyl)amide

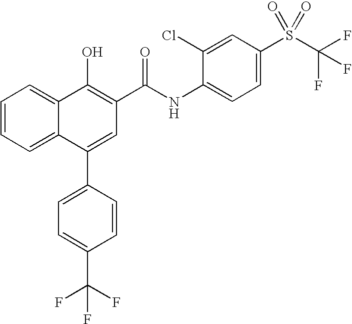

Step A: From 4-bromo-1-hydroxynaphthalene-2-carboxylic acid and 4-trifluoromethylphenylboronic acid.

Step B: From the product formed in step A and 2-chloro-4-trifluoromethanesulfonyl aniline

¹H NMR (DMSO-d₆): δ 7.66-7.80 (m, 5H) 7.92 (d, 2H) 8.07 (s, 1H) 8.21 (dd, 1H) 8.35 (d, 1H) 8.51 (dd, 1H) 8.67 (d, 1H) 11.74 (s, 1H); HPLC-MS (Method B): m/z=574 (M+1); Rₜ=3.02 min.

### Example 73

### General Procedure (A)

### 1-Hydroxy-4-(3-methoxyphenyl)naphthalene-2-carboxylic acid (2-chloro-4-trifluoromethanesulfonylphenyl)amide

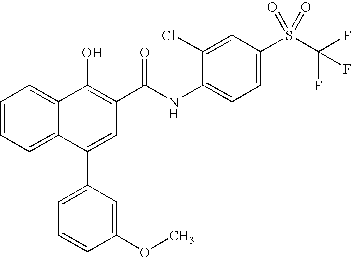

Step A: From 4-bromo-1-hydroxynaphthalene-2-carboxylic acid and 3-methoxyphenyl-boronic acid.

Step B: From the product formed in step A and 2-chloro-4-trifluoromethanesulfonyl aniline

¹H NMR (DMSO-d₆): δ 3.83 (s, 3H) 7.06 (m, 3H) 7.47 (dd, 1H) 7.67 (m, 2H) 7.84 (dd, 1H) 8.04 (s, 1H) 8.21 (dd, 1H) 8.37 (d, 1H) 8.47 (dd, 1H) 8.64 (d, 1H) 11.66 (s, 1H); HPLC-MS (Method B): m/z=536 (M+1); Rₜ=2.83 min.

### Example 74

### 4-Bromo-1-hydroxynaphthalene-2-carboxylic acid (2-chloro-4-trifluoromethanesulfonylphenyl)amide

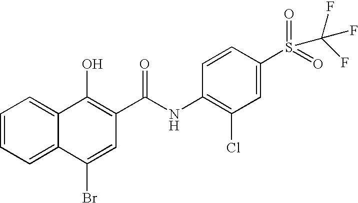

4-Bromo-1-hydroxynaphthalene-2-carboxylic acid and 2-chloro-4-trifluoromethanesulfonyl aniline were reacted as described in the General procedure (A), step B.

¹H NMR (DMSO-d₆): δ 7.72 (dd, 1H) 7.85 (dd, 1H) 8.10 (d, 1H) 8.20 (d, 1H) 8.34-8.39 (m, 2H) 8.45 (d, 1H) 8.73 (d, 1H) 11.97 (s, 1H); HPLC-MS (Method B): m/z=510 (M+1); Rₜ=2.73 min.

### Example 75

### General Procedure (A)

### 5-Bromo-3-tert-butyl-N-(2-chloro-4-trifluoromethanesulfonylphenyl)-2-hydroxybenzamide

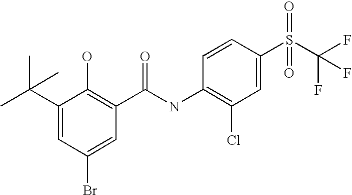

Step B: From 5-bromo-3-tert-butyl-2-hydroxybenzoic acid and 2-chloro-4-(trifluoromethanesulfonyl)aniline; ¹H NMR (DMSO-d₆): δ 1.38 (s, 9H), 7.53 (s, 1H), 8.10-8.23 (m, 3H), 8.37 (s, 1H), 11.01 (s, 1H), 12.62 (s, 1H) ppm; HPLC-MS (Method B): m/z: 514/516 (M+H), Rₜ=2.91 min.

### Example 76

### General Procedure (A)

### 5-tert-Butyl-3′,4′-difluoro-4-hydroxybiphenyl-3-carboxylic acid (2-chloro-4-trifluoromethanesulfonylphenyl)amide

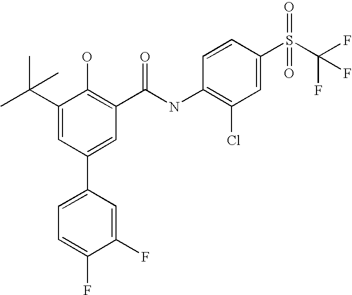

Step A: From 5-bromo-3-tert-butyl-2-hydroxybenzoic acid and 3,4-difluorophenylboronic acid.

Step B: From the product formed in step A and 2-chloro-4-(trifluoromethanesulfonyl)aniline;

¹H NMR (DMSO-d₆): δ 1.45 (s, 9H), 7.51-7.61 (m, 2H), 7.72 (s, 1H), 7.80-7.90 (m, 1H), 8.08-8.15 (m, 1H), 8.21-8.27 (m, 2H), 8.40 (s, 1H), 11.01 (s, 1H), 12.71 (s, 1H) ppm; HPLC-MS (Method B): m/z: 548 (M+H), Rₜ=3.03 min.

### Example 77

### General Procedure (A)

### 5-tert-Butyl-4′-fluoro-4-hydroxybiphenyl-3-carboxylic acid (2-chloro-4-trifluoromethanesulfonylphenyl)amide

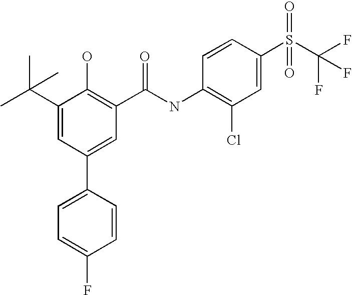

Step A: From 5-bromo-3-tert-butyl-2-hydroxybenzoic acid and 4-fluorophenylboronic acid.

Step B: From the product formed in step A and 2-chloro-4-(trifluoromethanesulfonyl)aniline;

¹H NMR (DMSO-d₆): δ 1.45 (s, 9H), 7.32 (t, J=8.84 Hz, 2H), 7.70 (s, 1H), 7.76 (m, 2H), 8.08-8.16 (m, 1H), 8.19-8.27 (m, 2H), 8.39 (s, 1H), 11.01 (s, 1H), 12.65 (s, 1H) ppm; HPLC-MS (Method B): m/z: 530 (M+H), Rₜ=3.01 min.

### Example 78

### General Procedure (A)

### 5-tert-Butyl-3′,5′-difluoro-4-hydroxybiphenyl-3-carboxylic acid (2-chloro-4-trifluoromethanesulfonylphenyl)amide (mixture with 5-bromo-3-tert-butyl-N-(2-chloro-4-trifluoromethanesulfonylphenyl)-2-hydroxybenzamide)

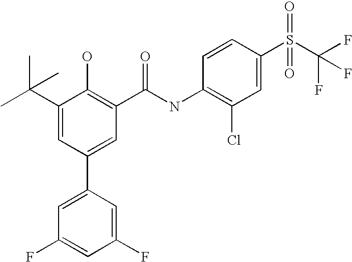

Step A: From 5-bromo-3-tert-butyl-2-hydroxybenzoic acid and 3,5-difluorophenylboronic acid.

Step B: From the product formed in step A and 2-chloro-4-(trifluoromethanesulfonyl)aniline;

¹H NMR (DMSO-d₆): δ 1.45 (s, 9H), 7.22 (m, 1H), 7.53 (d, J=7.58 Hz, 2H), 7.77 (s, 1H), 8.07-8.44 (m, 4H), 11.02 (s, 1H), 12.80 (s, 1H) ppm; HPLC-MS (Method B): m/z: 548 (M+H), Rₜ=3.04 min.

### Example 79

### General Procedure (A)

### 3,5-Di-tert-butyl-N-(2-chloro-4-trifluoromethanesulfonylphenyl)-2-hydroxybenzamide

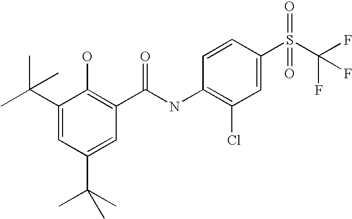

Step B: From 3,5-di-tert-butyl-2-hydroxybenzoic acid and 2-chloro-4-(trifluoromethanesulfonyl)aniline; ¹H NMR (DMSO-d₆): δ 1.32 (s, 9H), 1.40 (s, 9H), 7.50 (s, 1H), 7.87 (d, J=2.53 Hz, 1H), 8.11-8.25 (m, 2H), 8.37 (s, 1H), 10.84 (s, 1H), 12.41 (s, 1H) ppm; HPLC-MS (Method B): m/z: 492/494 (M+H), Rₜ=3.15 min.

### Example 80

### General Procedure (A)

### 3-tert-Butyl-N-(2-chloro-4-trifluoromethanesulfonylphenyl)-2-hydroxy-6-methylbenzamide

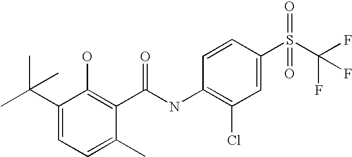

Step B: From 3-tert-butyl-2-hydroxy-6-methylbenzoic acid and 2-chloro-4-(trifluoromethanesulfonyl)aniline; ¹H NMR (DMSO-d₆): δ 1.37 (s, 9H), 2.31 (s, 3H), 6.73 (s, 1H), 7.16 (d, J=8 Hz, 1H), 8.10-8.32 (m, 2H), 8.58 (d, J=8 Hz, 1H), 9.01 (s, 1H), 10.44 (s, 1H) ppm; HPLC-MS (Method B): m/z: 450/452 (M+H), Rₜ=2.74 min.

### Example 81

### 1-Hydroxy-3-methylnaphthalene-2-carboxylic acid (2-chloro-4-trifluoromethanesulfonylphenyl)amide

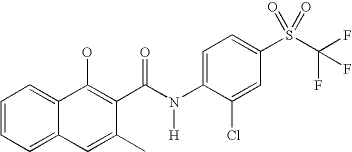

Boron trichloride 1M in dichloromethane (0.63 ml, 0.63 mmol) was added to a 0° C. dry flask under a nitrogen atmosphere, and 3-methylnaphthalen-1-ol (prepared according to the literature; M Watanabe et al; Chem. Pharm. Bull. 34 (7) 2810-2820, 1986) (100 mg, 0.63 mmol) dissolved in 3 ml of dry toluene was added. The reaction mixture was stirred for 10 minutes at 0° C., and then 2-chloro-1-isocyanato-4-trifluoromethanesulfonylbenzene (198.6 mg, 0.70 mmol) dissolved in 3 ml of dry toluene was added. The reaction mixture was stirred at 0° for 5 minutes, and then heated to 1200 for 1½ hours. The solvent was evaporated, and the residue was stirred in 50 ml of dichloromethane and 50 ml of 1M hydrochloric acid for 12 hours. The organic layer was separated, and the aqueous layer was extracted with 2×50 ml of dichloromethane. The combined organic layers were dried with sodium sulfate, the solvent was evaporated, and the product was finally crystallized from 3 ml of acetonitrile to give 150 mg of the title compound.

¹H NMR (DMSO-d₆): δ 10.58 (br. s, 1H) 10.19 (br.s, 1H) 8.67 (d, 1H) 8.29-8.24 (m, 2H) 8.21 (dd, 1H) 7.80 (d, 1H) 7.54 (dd, 1H) 7.48 (dd, 1H) 7.33 (s, 1H) 2.46 (s, 3H); HPLC-MS (Method B): m/z=444 (M+1); Rₜ=2.587 min.

### Example 82

### 4-Bromo-1-hydroxy-3-methylnaphthalene-2-carboxylic acid (2-chloro-4-trifluoromethanesulfonylphenyl)amide

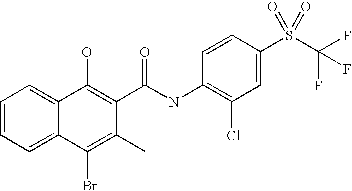

1-Hydroxy-3-methylnaphthalene-2-carboxylic acid (2-chloro-4-trifluoromethanesulfonylphenyl)amide (50 mg, 0.113 mmol) was dissolved in 3 ml of dichloromethane, and the solution was cooled to 0°. Bromine (0.006 ml, 0.113 mmol) dissolved in 0.5 ml dichloromethane was added, and the mixture was stirred for 45 minutes at 0° and then at room temperature for 2 hours. The solvent was evaporated, and the residue was crystallized from 2.5 ml of acetonitrile to give 25 mg of the title compound.

¹H NMR (DMSO-d₆): δ 2.56 (s, 3H) 7.58 (dd, 1H) 7.72 (dd, 1H) 8.18-8.34 (m, 4H) 8.66 (d, 1H) 10.34 (br. s, 1H) 10.79 (br.s, 1H); HPLC-MS (Method B): m/z=524 (M+2); Rₜ=2.65 min.

### Example 83

### General Procedure (A)

### 3-Bromo-5-tert-butyl-6-hydroxy-2-methyl-N-(2-nitro-4-trifluoromethanesulfonylphenyl)-benzamide

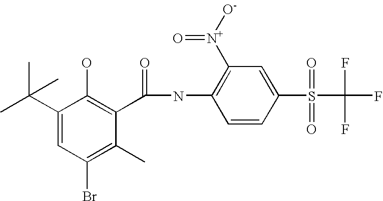

Step B: From 3-bromo-5-tert-butyl-6-hydroxy-2-methyl-benzoic acid and 2-nitro-4-trifluoromethanesulfonyl aniline.

¹H NMR (DMSO-d₆): δ ppm 1.35 (s, 9H) 2.33 (s, 3H) 7.42 (s, 1H) 8.28 (d, J=8.67 Hz, 1H) 8.50 (d, J=8.67 Hz, 1H) 8.58 (d, J=1.88 Hz, 1H) 9.24 (s, 1H) 11.46 (s, 1H); HPLC-MS (Method B): m/z=561, 563 (M+1); Rₜ=2.75 min.

### Example 84

### General Procedure (A)

### 5-tert-Butyl-3′-fluoro-4-hydroxy-2-methyl-biphenyl-3-carboxylic acid (2-chloro-4-trifluoromethanesulfonyl-phenyl)-amide

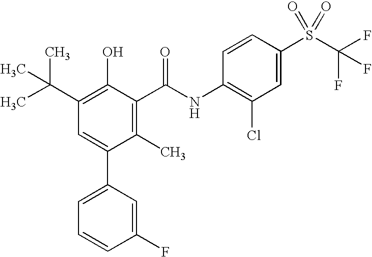

Step A: From 3-bromo-5-tert-butyl-6-hydroxy-2-methyl-benzoic acid and 3-fluoro phenyl-boronic acid.

Step B: From the product formed in step A and 2-chloro-4-trifluoromethanesulfonyl aniline.

¹H NMR (DMSO-d₆): δ ppm 1.35-1.44 (s, 9H) 2.17 (s, 3H) 7.05-7.26 (m, 3H) 7.41-7.55 (m, 1H) 8.20 (d, J=8.67 Hz, 1H) 8.25 (d, J=1.88 Hz, 1H) 8.63 (d, J=8.67 Hz, 1 H) 9.08 (s, 1H) 10.62 (s, 1H); HPLC-MS (Method B): m/z=544, 546 (M+1); Rₜ=2.92 min.

### Example 85

### General Procedure (A)

### 5′-tert-Butyl-3′-(2-chloro-4-trifluoromethanesulfonyl-phenylcarbamoyl)-4′-hydroxy-2′-methyl-biphenyl-3-carboxylic acid methyl ester

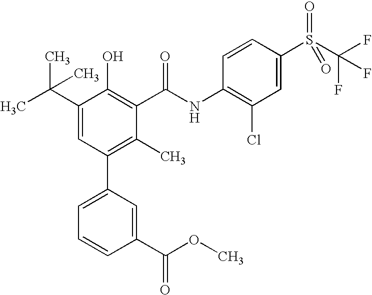

Step A: From 3-Iodo-5-tert-butyl-6-hydroxy-2-methyl-benzoic acid and 3-methoxycarbonylphenylboronic acid.

Step B: From the product formed in step A and 2-chloro-4-trifluoromethanesulfonyl aniline.

HPLC-MS (Method B): m/z=583, 585 (M+1); Rₜ=2.84 min.

### Example 86

### 3-tert-Butyl-N-(3-chloro-4-trifluoromethanesulfonyl-phenyl)-5-fluoro-2-hydroxy-6-methyl-benzamide

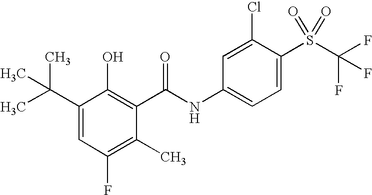

Step A: Sodium hydride (2.8 g, 60% dispersion in oil) was carefully washed twice with petroleum ether, suspended in dry THF (150 mL), and the suspension was cooled to 0° C. under nitrogen. A solution of 2-tert-butyl-4-fluoro-5-methylphenol (8.7 g, 47.7 mmol) in dry THF (150 mL) was then added dropwise, and the mixture was stirred at room temperature for 2 h. A solution of chloromethyl methyl ether (10.9 mL, 143 mmol) in dry THF (150 mL) was added dropwise, and the resulting suspension was stirred at room temperature for 1 h and evaporated to dryness. The residue was partitioned between diethyl ether (200 mL) and 1 N NaOH (200 mL). The organic layer was washed with brine (50 mL), dried over MgSO₄ and concentrated in vacuo to give 10.68 g (99%) of 1-tert-butyl-5-fluoro-2-methoxymethoxy-4-methylbenzene as a yellow oil; ¹H NMR (CDCl₃) δ 1.36 (s, 9H) 2.21 (d, J=1.88 Hz, 3H) 3.49 (s, 3H) 5.17 (s, 2H) 6.91 (d, J=7.16 Hz, 1H) 6.93 (d, J=12.06 Hz, 1H) ppm.

Step B: A solution of 5 g of the product formed in step A in dry THF (25 mL) was cooled to −78° C. under nitrogen. n-Butyllithium (n-BuLi) (15.2 mL, 1.6 M in hexane) was added dropwise over 5 min, and the reaction mixture was stirred at 0° C. for 1.5 h. The mixture was then transferred to a flask containing powdered, solid carbon dioxide by cannulation over a period of 5 min. The solid which formed was triturated with petroleum ether followed by diethyl ether to give 5.13 g (84%) of 3-tert-butyl-5-fluoro-2-methoxymethoxy-6-methylbenzoic acid as the lithium salt; HPLC-MS (Method B): m/z: 293 (M+Na), Rₜ=2.08.

Step C: A solution of 2 g of the product formed in step B in water (25 mL) was acidified by the addition of 1 M HCl (20 mL) and extracted with dichloromethane (3×25 mL). The combined organic phase was dried over Na₂SO₄ and evaporated to dryness. The resulting free acid was dissolved in anhydrous methanol (25 mL), and a few drops of boron trifluoride-diethyl etherate were added. The solution was stirred at room temperature for 65 h and evaporated to dryness. The residue was triturated with petroleum ether to give 827 mg (50%) of 3-tert-butyl-5-fluoro-2-hydroxy-6-methylbenzoic acid; ¹H NMR (DMSO-d₆) δ 1.35 (s, 9H), 2.34 (d, J=2.53 Hz, 3H), 7.15 (d, J=11.62 Hz, 1H), ca. 12.5 (br. s., 1H) ppm, one proton is hidden under the water peak; HPLC-MS (Method B): m/z: 227 (M+H), Rₜ=2.36 min.

Step D: From the product formed in step C and 3-chloro-4-(trifluoromethylsulfonyl)aniline by analogy with step B in general procedure D; ¹H NMR (DMSO-d₆): δ 1.40 (s, 9H) 2.45 (d, J=2.02 Hz, 3H) 7.16 (d, J=11.62 Hz, 1H) 7.68-7.73 (m, 2H) 8.08 (d, J=2.02 Hz, 1H) 8.18 (d, J=8.59 Hz, 1H) 9.29 (br. s., 1H) ppm; HPLC-MS (Method B): m/z: 490/492 (M+Na), 468/470 (M+H), Rₜ=2.69 min.

### Example 87

### 3-tert-Butyl-5-chloro-N-(2-dimethylamino-4-trifluoromethanesulfonylphenyl)-2-hydroxy-6-methylbenzamide

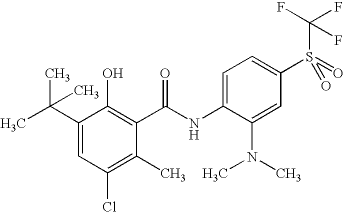

From 3-tert-butyl-5-chloro-2-hydroxy-6-methylbenzoic acid and N-{2-amino-5-[(trifluoromethyl)sulfonyl]phenyl}-N,N-dimethylamine by analogy with step B in general procedure D, except that the mixture was stirred for 48 h; ¹H NMR (CDCl₃): δ 1.41 (s, 9H) 2.57 (s, 3H) 2.71 (s, 6H) 7.41 (s, 1H) 7.85-7.97 (m, 2H) 8.88 (d, J=8.67 Hz, 1H) 9.47 (br. s., 1H) 9.88 (br. s., 1H) ppm; HPLC-MS (Method B): m/z: 493/495 (M+H), Rₜ=2.94 min.

### Example 88

### 3-tert-Butyl-5-chloro-N-(2-dipropylamino-4-trifluoromethanesulfonylphenyl)-2-hydroxy-6-methylbenzamide

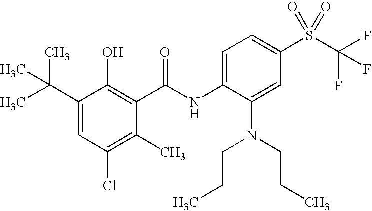

From 3-tert-butyl-5-chloro-2-hydroxy-6-methylbenzoic acid and N-{2-amino-5-[(trifluoromethyl)sulfonyl]phenyl}-N,N-dipropylamine by analogy with step B in general procedure D, except that the mixture was stirred for 48 h; ¹H NMR (CDCl₃): δ 0.79 (t, J=7.00 Hz, 6H) 1.28-1.39 (m, 4H) 1.40 (s, 9H) 2.55 (s, 3H) 2.81-2.89 (m, 4H) 7.39 (s, 1H) 7.83 (d, J=2.26 Hz, 1H) 7.90 (dd, J=8.67, 2.00 Hz, 1H) 8.84 (d, J=8.67 Hz, 1H) 9.48 (br. s., 1H) 9.69 (s, 1H) ppm; HPLC-MS (Method B): m/z: 550/552 (M+H), Rₜ=3.16 min.

### Example 89

### 3-tert-Butyl-(2-chloro-4-trifluoromethanesulfonylphenyl)-2-hydroxy-6-methyl-5-(morpholine-4-sulfonyl)-benzamide

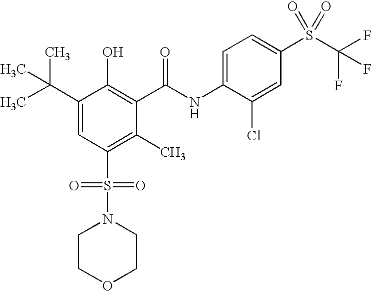

A suspension of 3-tert-butyl-N-(2-chloro-4-trifluoromethanesulfonylphenyl)-2-hydroxy-6-methylbenzamide (200 mg, 0.45 mmol) in dichloromethane (5 mL) was cooled to 0° C. Chlorosulfonic acid (178 μL, 2.7 mmol) was added, and the mixture was stirred at room temperature for 3.5 h. The mixture was poured into ice water, and the organic phase was isolated, dried over Na₂SO₄ and filtered. To the resulting solution was added morpholine (100 μL, 1.1 mmol), and the mixture was stirred overnight at room temperature. The mixture was concentrated in vacuo and subjected to column chromatography, giving 21 mg (8%) of the title compound; ¹H NMR (DMSO-d₆): δ 1.44 (s, 9H) 2.84 (s, 3H) 3.18-3.24 (m, 4H) 3.70-3.79 (m, 4H) 7.97-8.08 (m, 2H) 8.12 (d, J=2.26 Hz, 1H) 8.44 (br. s., 1H) 8.99 (d, J=8.67 Hz, 1H) 9.54 (br. s., 1H) ppm; HPLC-MS (Method B): m/z: 621/623 (M+23), 599/601 (M+H), Rₜ=2.27.

### Example 90

### 3-tert-Butyl-N-(2-chloro-4-trifluoromethanesulfonylphenyl)-5-dimethylsulfamoyl-2-hydroxy-6-methyl-benzamide

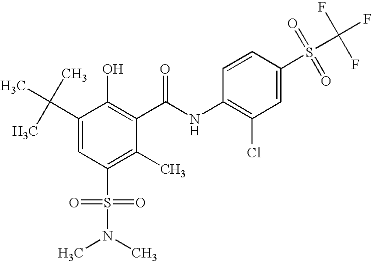

Step A: 3-tert-Butyl-N-(2-chloro-4-trifluoromethanesulfonylphenyl)-2-hydroxy-6-methylbenzamide (500 mg, 1.10 mmol) was added over 5 min in small portions to a solution of chlorosulfonic acid (443 μL, 6.64 mmol) in dichloromethane (20 mL) with stirring at 0° C. The mixture was stirred at room temperature for 4 h and poured into ice water. The organic phase was isolated, dried over Na₂SO₄ and filtered. The sticky solid was triturated with water, isolated by filtration, and dried to give 439 mg (72%) of beige crystals of crude 5-tert-butyl-3-(2-chloro-4-trifluoromethanesulfonyl-phenylcarbamoyl)-4-hydroxy-2-methyl-benzenesulfonyl chloride.

Step B: To a solution of the product formed in step A (200 mg, 0.36 mmol) in dichloromethane (3 mL) was added dimethylamine hydrochloride (74 mg, 0.91 mmol) and triethylamine (127 μl, 0.91 mmol), and the mixture was stirred for 1 h at room temperature. The mixture was then concentrated in vacuo and subjected to column chromatography, giving 85 mg (42%) of the pure title compound; ¹H NMR (CDCl₃): δ 1.43 (s, 9H) 2.82 (s, 3H) 2.84 (s, 6H) 7.99 (s, 1H) 8.04 (dd, J=9.04, 2.26 Hz, 1H) 8.11 (d, J=2.26 Hz, 1H) 8.43 (br. s., 1H) 8.99 (d, J=9.04 Hz, 1H) 9.57 (s, 1H) ppm; HPLC-MS (Method B): m/z: 579/581 (M+23), 557/559 (M+H), Rₜ=2.39 min.

### Example 91

### 3-tert-Butyl-N-(2-chloro-4-trifluoromethanesulfonylphenyl)-5-sulfamoyl-2-hydroxy-6-methyl-benzamide

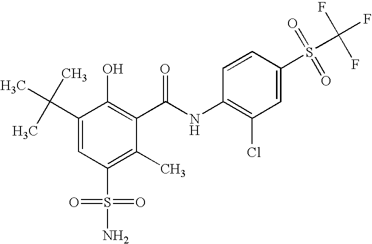

To a solution of crude 5-tert-butyl-3-(2-chloro-4-trifluoromethanesulfonyl-phenylcarbamoyl)-4-hydroxy-2-methyl-benzenesulfonyl chloride (200 mg, 0.36 mmol) in dichloromethane (3 mL) was added ammonia (1.8 mL of a 0.5 M solution in dioxane), and the mixture was stirred for 1 h at room temperature. The mixture was concentrated in vacuo and purified by column chromatography to give 34 mg (18%) of the pure title compound; ¹H NMR (CDCl₃): δ 1.42 (s, 9H) 2.90 (s, 3H) 4.00 (very br. s., 2H) 8.04 (dd, J=9.04, 1.88 Hz, 1H) 8.11 (d, J=1.88 Hz, 1H) 8.15 (s, 1H) 8.45 (br. s., 1H) 8.99 (d, J=8.67 Hz, 1H) 9.54 (br. s., 1H) ppm; HPLC-MS (Method B): m/z: 551/553 (M+23), 529/531 (M+H), Rₜ=2.14 min.

### Example 92

### 3-tert-Butyl-N-(2-chloro-4-trifluoromethanesulfonyl-phenyl)-2-hydroxy-6-methoxy-benzamide

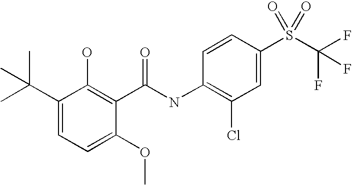

Step A: 3-tert-butyl-2-hydroxy-6-methoxy-benzoic acid was prepared in a sulfuric acid catalyzed t-butylation of 2-hydroxy-6-methoxy-benzoic acid with t-butanol in glacial acetic acid by standard methods; ¹H NMR (CDCl₃): δ 12.96 (s, 1H), 11.6 (br, 1H), 7.40 (d, J=8.6 Hz, 1H), 7.42 (d, J=8.6 Hz, 1H), 4.03 (s, 3H), 1.37 (s, 9H); HPLC-MS (Method B): m/z 207 (M-OH), 225 (M+H), 247 (M+Na), Rₜ 2.07 min. The crude product was practically pure and was used without purification.

Step B: (General procedure (E)). From the product formed in step A and 2-chloro-4-trifluoromethanesulfonyl-aniline, mp 198-199° C.; ¹H NMR (CDCl₃): 613.71 (s, 1H), 11.50 (s, 1H), 8.98 (d, J=9.1 Hz, 1H), 8.09 (d, J=2.5 Hz, 1H), 7.97 (dd, J=2.0 and 8.6 Hz, 1H), 7.41 (d, J=8.6 Hz, 1H), 6.43 (d, J=9.1 Hz, 1H), 4.07 (s, 3H), 1.41 (s, 9H); HPLC-MS (Method B): m/z 466/468 (M+H), Rₜ 3.05 min.

### Example 93

### 3-tert-Butyl-N-(2-chloro-4-trifluoromethanesulfonyl-phenyl)-2-hydroxy-5-methoxy-6-methyl-benzamide

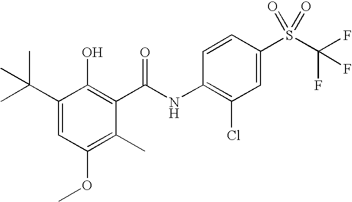

Step A: 2-tert-butyl-4-methoxy-5-methyl-phenol was prepared in a sulfuric acid catalyzed t-butylation of 4-methoxy-3-methyl-phenol with t-butanol in glacial acetic acid by standard methods; yield 93%; ¹H NMR (CDCl₃): δ 6.78 (s, 1H), 6.47 (s, 1H), 4.47 (br s, 1H), 3.79 (s, 3H), 2.14 (s, 3H), 1.40 (s, 9H). The crude oily product was practically pure and was used without purification.

Step B: (General procedure (C)). From the product formed in step A and 2-chloro-1-isocyanato-4-trifluoromethanesulfonyl-benzene; off-white crystals, mp 121-122° C.; ¹H NMR (CDCl₃): δ 9.08 (s, 1H), 9.02 (d, J=9.1 Hz, 1H), 8.48 (s, 1H), 8.08 (d, J=2 Hz, 1H), 8.00 (dd, J=9.1 and 2 Hz, 1H), 7.06 (s, 1H), 3.85 (s, 3H), 2.46 (s, 3H), 1.43 (s, 9H); HPLC-MS (Method B): m/z 480/482 (M+H), 502/504 (M+Na), Rₜ 2.82 min.

### Example 94

### General Procedure (C)

### 6-tert-Butyl-5-hydroxy-2,3-dihydro-benzofuran-4-carboxylic acid (2-chloro-4-trifluoromethanesulfonyl-phenyl)-amide

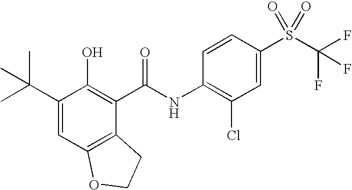

From 6-tert-butyl-2,3-dihydro-benzofuran-5-ol and 2-chloro-1-isocyanato-4-trifluoromethanesulfonyl-benzene; mp 201-202° C.; ¹H NMR (CDCl₃): δ 11.58 (s, 1H), 8.95 (d, J=9 Hz, 1H), 8.65 (s, 1H), 8.11 (d, J=1.5 Hz, 1H), 8.01 (dd, J=9 and 1.5 Hz, 1H), 7.09 (s, 1H), 4.65 (t like m, 2H), 3.64 (t like m, 2H), 1.41 (s, 9H); HPLC-MS (Method B): m/z 478/480 (M+H); 500/502 (M+Na), Rₜ 2.84 min.

### Example 95

### 3-tert-Butyl-N-(2-chloro-4-trifluoromethanesulfonyl-phenyl)-2,5-dihydroxy-6-methylbenzamide

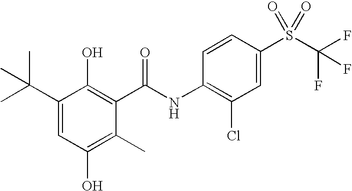

A stirred solution of 3-tert-butyl-N-(2-chloro-4-trifluoromethanesulfonyl-phenyl)-2-hydroxy-5-methoxy-6-methyl-benzamide (0.150 g, 0.31 mmol) in dry dichloromethane (2 ml) under nitrogen was cooled to 0° C. A 1 M solution of boron tribromide in dichloromethane (0.34 ml, 1.1 equivalent) was added dropwise and the mixture was allowed to stand at room temperature overnight and then washed with saturated aqueous sodium bicarbonate (10+2×5 ml) and water (10 ml), dried over sodium sulfate and filtered, and the solvent was evaporated to give the title compound; mp 163-166° C.; ¹H NMR (CDCl₃): δ 9.20 (s, 1H), 9.02 (d, J=9.1 Hz, 1H), 8.47 (brs, 1H), 8.10 (d, J=2.5 Hz, 1H), 8.02 (dd, J=9.1 and 2 Hz, 1H), 6.95 (s, 1H), 4.62 (br s, 1H), 2.50 (s, 3H), 1.41 (s, 9H); HPLC-MS (Method B): m/z 466 (M+H), 468 (M+2+H), 488 (M+Na), 490 (M+2+Na), Rₜ 2.34 min.

### Example 96

### 3-tert-Butyl-N-(2-chloro-4-trifluoromethanesulfonyl-phenyl)-2,5-dihydroxy-6-methoxy-benzamide

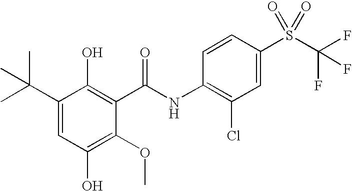

To a stirred solution of 3-tert-butyl-N-(2-chloro-4-trifluoromethanesulfonyl-phenyl)-2-hydroxy-5-methoxy-benzamide (0.200 g, 0.43 mmol) in acetonitrile (5 ml) was added a solution of ammonium cerium (IV) nitrate (0.71 g, 1.3 mmol) in water (1 ml). A voluminous precipitate formed and to facilitate the stirring, more acetonitrile (5 ml) was added. After 2 h water (20 ml) was added and the mixture was filtered. The filter cake was rinsed with water and dried. Yield 0.198 g of crude hydroquinone (5-tert-butyl-3,6-dioxo-cyclohexa-1,4-dienecarboxylic acid (2-chloro-4-trifluoromethanesulfonyl-phenyl)-amide) as an orange solid. The crude product (0.157 g) was heated with boiling methanol (15 ml) for some minutes and allowed to stand at room temperature overnight. A precipitate was filtered off and the solvent was removed from the filtrate in vacuo. The residue was dissolved in a 1:2 mixture of ethyl acetate—heptane (5 ml) and filtered through a short column of silica gel. Removal of the solvent from the filtrate gave the title compound as a yellow oil, which crystallized by trituration with pentane; yellow crystals, mp 171-175° C.; ¹H NMR (DMSO-d₆): δ 12.54 (s, 1H), 11.33 (s, 1H), 9.01 (d, J=9.10 Hz, 1H), 8.10 (d, J=2.02 Hz, 1H), 7.98 (dd, J=2.02; 9.10 Hz, 1H), 7.20 (s, 1H), 4.95 (s, 1H), 3.97 (s, 3H), 1.41 (s, 9H); ¹³C NMR (CDCl₃): δ 169.16 (C═O), 156.81, 142.40, 141.87, 139.43, 136.59, 131.55, 130.85, 125.76, 124.00, 122.06, 119.70 (CF₃, J=325.68 Hz), 106.34, 62.66, 35.05, 29.20; HPLC-MS (Method B): m/z 482 (M+H), 484 (M+2+H), 504 (M+Na), 506 (M+2+Na), Rₜ 2.71 min.; C—H correlation spectra ruled out isomeric structures.

### Example 97

### 3-tert-Butyl-N-(2-chloro-4-trifluoromethanesulfonyl-phenyl)-2,6-dihydroxy-5-methoxy-benzamide

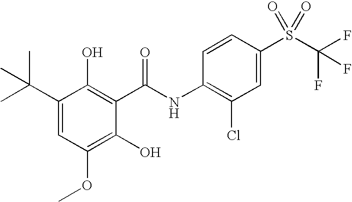

Step A: 2-tert-butyl-4,5-dimethoxy-phenol was prepared in a sulfuric acid catalyzed t-butylation of 3,4-dimethoxy-phenol with t-butanol in glacial acetic acid by standard methods; purification by preparative HPLC gave a pure product as a yellow oil; ¹H NMR (CDCl₃): 6.83 (s, 1H), 6.32 (s, 1H), 4.72 (s, 1H), 3.84 (s, 3H), 3.79 (s, 3H), 1.39 (s, 9H).

Step B: (General procedure (C)). From the product formed in step A and 2-chloro-1-isocyanato-4-trifluoromethanesulfonyl-benzene; in this case a selective demethylation α-cured, and only the dihydroxy-monomethoxy-derivative was isolated; mp 170-171° C.; ¹H NMR (CDCl₃): δ 12.77 (s, 1H), 11.14 (s, 1H, NH), 8.97 (d, J=9.10 Hz, 1H), 8.09 (d, J=2.02 Hz, 1H), 7.97 (dd, 1H), 7.15 (s, 1H), 7.02 (s, 1H), 3.93 (s, 3H), 1.42 (s, 9H); ¹³C NMR (CDCl₃) δ 169.63, 156.61, 142.64, 142.14, 137.04, 131.44, 130.60, 129.70, 125.49, 124.40, 122.20, 119.71 (q, J=326 Hz), 116.94, 102.28, 57.44, 34.84, 29.38; HPLC-MS (Method B): m/z 482/484 (M+H), Rₜ 2.86 min.; C—H correlation spectra ruled out isomeric structures.

### Example 98

### General Procedure (C)

### N-(2-Chloro-4-trifluoromethanesulfonyl-phenyl)-2-hydroxy-3-isopropyl-6-methyl-5-methylsulfanyl-benzamide

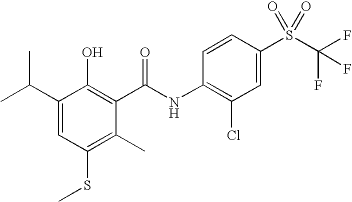

From 2-isopropyl-5-methyl-4-methylsulfanyl-phenol and 2-chloro-4-trifluoromethanesulfonyl-aniline, ¹H NMR (CD₃OD): δ 8.78-8.63 (br m, 1H), 8.17 (d, J=2.0 Hz, 1H), 8.06 (br d, J=9.1 Hz, 1H), 7.30 (s, 1H), 4.9 (s, water, OH, NH), 3.35, 3.23 (m, methanol+methin CH), 2.43 (s, 3H), 2.38 (s, 3H), 1.25 (d, J=7.1 Hz, 6H); HPLC-MS (Method B): m/z 482/484 (M+H), 504/506 (M+Na), Rₜ 2.58 min.

### Example 99

### 3-tert-Butyl-N-(2-chloro-4-trifluoromethanesulfonyl-phenyl)-6-ethyl-2-hydroxy-5-methoxy-benzamide

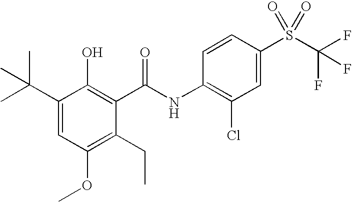

Step A: 2-tert-butyl-5-ethyl-4-methoxy-phenol was prepared in a sulfuric acid catalyzed t-butylation of 3-ethyl-4-methoxy-phenol with t-butanol in glacial acetic acid by standard methods; yield 98%; ¹H NMR (CDCl₃): δ 6.79 (s, 1H), 6.49 (s, 1H), 4.51 (br s, 1H), 3.79 (s, 3H), 2.55 (q, J=7.6 Hz, 2H), 1.40 (s, 9H), 1.16 (t, J=7.6 Hz, 3H). The crude oily product was practically pure and was used without purification.

Step B: (General procedure (C)). From the product formed in step A and 2-chloro-1-isocyanato-4-trifluoromethanesulfonyl-benzene; ¹H NMR (CDCl₃): δ 8.87 (d, J=9.1 Hz, 1H), 8.30 (s, 1H), 8.00 (d, J=2.02 Hz, 1H), 7.90 (dd, J=9.1 and 2.02 Hz, 1H), 6.94 (s, 1H), 3.76 (s, 3H), 2.72 (q, 2H), 1.34 (s, 9H), 1.19 (t, 3H); HPLC-MS (Method B): m/z 494/496 (M+H), 516/518 (M+Na), Rₜ 2.72 min.

### Example 100

### General Procedure (E)

### 3,5-Di-tert-butyl-N-(2-chloro-4-trifluoromethanesulfonyl-phenyl)-2,6-dihydroxy-benzamide

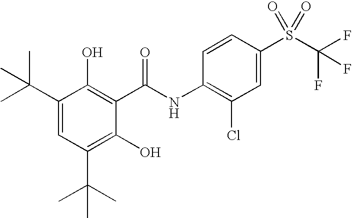

From 3,5-di-tert-butyl-2,6-dihydroxy-benzoic acid and 2-chloro-4-trifluoromethanesulfonyl-aniline, mp 149-150° C.; ¹H NMR (CDCl₃): δ 11.77 (s, 1H), 8.29-8.26 (br, 1H), 8.13-8.07 (br d-like, 1H), 7.87-7.80 (br d-like, 1H), 7.65 (s, 1H), 1.43 (s, 18 H); HPLC-MS (Method B): m/z 508/510 (M+H), Rₜ 3.19 min.

### Example 101

### General Procedure (E)

### 3-tert-Butyl-N-(2-chloro-4-trifluoromethanesulfonyl-phenyl)-5-cyano-2-hydroxy-6-methyl-benzamide

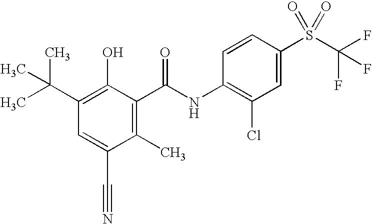

From 3-tert-butyl-5-cyano-2-hydroxy-6-methyl-benzoic acid and 2-chloro-4-trifluoromethanesulfonyl aniline; ¹H NMR (CDCl₃): δ 1.41 (s, 9H) 2.84 (s, 3H) 7.65 (s, 1H) 8.04 (d, J=8.59 Hz, 1H) 8.12 (s, 1H) 8.47 (s, 1H) 8.97 (d, J=8.59 Hz, 1H) 10.55 (s, 1H); HPLC-MS (Method B): m/z=497/499 (M+23), 475/477 (M+1); Rₜ=2.42 min.

### Example 102

### 3-tert-Butyl-N-(2-chloro-4-trifluoromethanesulfonyl-phenyl)-2-hydroxy-6-methyl-5-(4-methyl-piperazine-1-sulfonyl)-benzamide

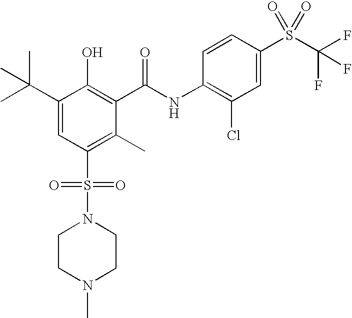

To a suspension of 5-tert-butyl-3-(2-chloro-4-trifluoromethanesulfonyl-phenylcarbamoyl)-4-hydroxy-2-methyl-benzenesulfonyl chloride (200 mg, 0.36 mmol) in dichloromethane (3 mL) was added N-methylpiperazine (101 μL, 0.91 mmol), and the mixture was stirred for 1.5 h at room temperature. The mixture was concentrated in vacuo and purified by column chromatography to give the pure title compound which was isolated as colourless crystals of the hydrochloride salt; ¹H NMR (MeOD): δ 1.45 (s, 9H) 2.61 (s, 3H) 2.93 (s, 3H) 3.04-3.27 (m, 4H) 3.50-3.67 (m, 2H) 3.76-3.94 (m, 2H) 7.96 (s, 1H) 8.08 (dd, J=8.67, 1.88 Hz, 1H) 8.19 (d, J=1.88 Hz, 1H) 8.74 (d, J=8.67 Hz, 1H); HPLC-MS (Method B): m/z=612/614 (M+1); Rₜ=1.77 min.

### Example 103

### General Procedure (E)

### 3-tert-Butyl-N-(2-cyano-4-trifluoromethanesulfonyl-phenyl)-5-fluoro-2-hydroxy-6-methyl-benzamide

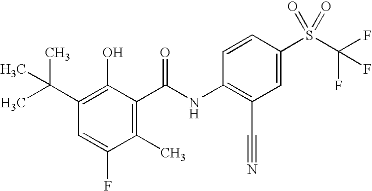

From 3-tert-butyl-5-fluoro-2-hydroxy-6-methyl-benzoic acid and 2-amino-5-trifluoromethanesulfonyl-benzonitrile; ¹H NMR (CDCl₃): δ 1.41 (s, 9H) 2.56 (d, J=2.02 Hz, 3H) 7.21 (d, J=11.62 Hz, 1H) 8.21-8.34 (m, 2H) 8.47 (br. s., 1H) 9.08 (d, J=8.59 Hz, 1H) 9.61 (s, 1H); HPLC-MS (Method B): m/z=481 (M+23), 459 (M+1); Rₜ=2.46 min.

### Example 104

### General Procedure (F)

### 3-tert-Butyl-5-fluoro-2-hydroxy-N-(2-methoxy-4-trifluoromethanesulfonyl-phenyl)-6-methyl-benzamide

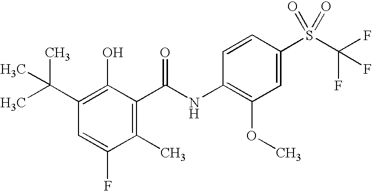

From 3-tert-butyl-5-fluoro-2-hydroxy-6-methyl-benzoic acid and 2-methoxy-4-trifluoromethanesulfonyl aniline; ¹H NMR (CDCl₃): δ 1.40 (s, 9H) 2.48 (d, J=2.53 Hz, 3H) 4.03 (s, 3H) 7.15 (d, J=11.62 Hz, 1H) 7.48 (s, 1H) 7.76 (dd, J=8.59, 2.02 Hz, 1H) 8.59 (br. s., 1H) 8.86 (d, J=8.59 Hz, 1H) 10.06 (s, 1H); HPLC-MS (Method B): m/z=464 (M+1); Rₜ=2.63 min.

### Example 105

### General Procedure (F)

### 3-tert-Butyl-5-fluoro-2-hydroxy-6-methyl-N-(2-methyl-4-trifluoromethanesulfonylphenyl)-benzamide

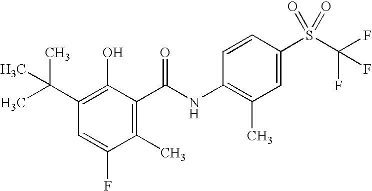

From 3-tert-butyl-5-fluoro-2-hydroxy-6-methyl-benzoic acid and 2-methyl-4-trifluoromethanesulfonyl aniline; ¹H NMR (CDCl₃): δ 1.41 (s, 9H) 2.45 (s, 3H) 2.50 (d, J=2.53 Hz, 3H) 7.16 (d, J=11.62 Hz, 1H) 7.66 (br. s., 1H) 7.89 (d, J=2.02 Hz, 1H) 7.97 (dd, J=8.59, 2.02 Hz, 1H) 8.65 (d, J=8.59 Hz, 1H) 9.67 (s, 1H); HPLC-MS (Method B): m/z=470 (M+23), 448 (M+1); Rₜ=2.58 min.

### Example 106

### General Procedure (F)

### N-(2-Bromo-4-trifluoromethanesulfonyl-phenyl)-3-tert-butyl-5-fluoro-2-hydroxy-6-methyl-benzamide

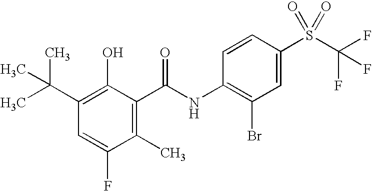

From 3-tert-butyl-5-fluoro-2-hydroxy-6-methyl-benzoic acid and 2-bromo-4-trifluoromethanesulfonyl aniline; ¹H NMR (CDCl₃): δ 1.41 (s, 9H) 2.55 (d, J=2.53 Hz, 3H) 7.18 (d, J=11.62 Hz, 1H) 8.05 (dd, J=8.59, 2.02 Hz, 1H) 8.25 (d, J=2.02 Hz, 1H) 8.48 (br. s., 1H) 8.97 (d, J=8.59 Hz, 1H) 9.70 (s, 1H); HPLC-MS (Method B): m/z=512/514 (M+1); Rₜ=2.64 min.

### Example 107

### General Procedure (F)

### 3-Acetyl-5-tert-butyl-N-(2-chloro-4-trifluoromethanesulfonyl-phenyl)-6-hydroxy-2-methyl-benzamide

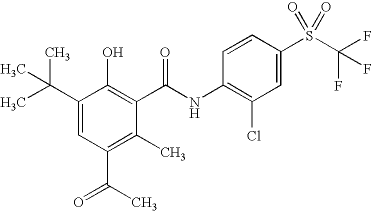

From 5-acetyl-3-tert-butyl-2-hydroxy-6-methyl-benzoic acid and 2-chloro-4-trifluoromethanesulfonyl aniline; ¹H NMR (CDCl₃): δ 1.45 (s, 9H) 2.60 (s, 3H) 2.74 (s, 3H) 7.81 (s, 1H) 8.02 (dd, J=8.59, 2.02 Hz, 1H) 8.10 (d, J=2.02 Hz, 1H) 8.48 (br. s., 1H) 9.01 (d, J=8.59 Hz, 1H) 9.77 (s, 1H); HPLC-MS (Method B): m/z=514/516 (M+23), 492/494 (M+1); Rₜ=2.36 min.

### Example 108

### General Procedure (F)

### 3-Acetyl-5-tert-butyl-6-hydroxy-N-(2-methoxy-4-trifluoromethanesulfonyl-phenyl)-2-methyl-benzamide

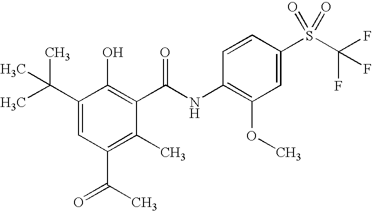

From 5-acetyl-3-tert-butyl-2-hydroxy-6-methyl-benzoic acid and 2-methoxy-4-trifluoromethanesulfonyl aniline; ¹H NMR (CDCl₃): δ 1.44 (s, 9H) 2.60 (s, 3H) 2.68 (s, 3H) 4.02 (s, 3H) 7.47 (s, 1H) 7.71-7.85 (m, 2H) 8.57 (br. s., 1H) 8.87 (d, J=8.59 Hz, 1H) 10.04 (s, 1H); HPLC-MS (Method B): m/z=510 (M+23), 488 (M+1); Rₜ=2.49 min.

### Example 109

### General Procedure (F)

### 3-tert-Butyl-5-chloro-N-(2-cyano-4-trifluoromethanesulfonyl-phenyl)-2-hydroxy-6-methyl-benzamide

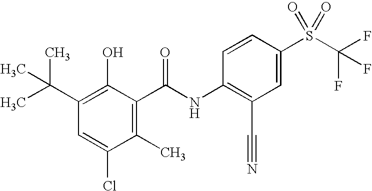

From 3-tert-butyl-5-chloro-2-hydroxy-6-methyl-benzoic acid and 2-amino-5-trifluoromethanesulfonyl-benzonitrile; ¹H NMR (DMSO-d₆): δ 1.38 (s, 9H) 2.13 (s, 3H) 7.30 (s, 1H) 8.05 (d, J=8.59 Hz, 1H) 8.42 (dd, J=8.59, 2.02 Hz, 1H) 8.70 (d, J=2.02 Hz, 1H) 9.16 (br. s., 1H) 13.09 (br. s., 1H); HPLC-MS (Method B): m/z=497/499 (M+23), 474/476 (M+1); Rₜ=2.47 min.

### Example 110

### 3-tert-Butyl-N-(2-chloro-4-trifluoromethanesulfonyl-phenyl)-2-hydroxy-6-methyl-5-nitro-benzamide

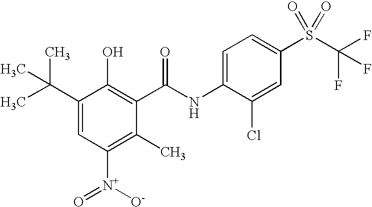

A solution of fuming nitric acid (0.26 mL, 6.2 mmol) in acetic acid (2 mL) was added dropwise to a solution of 3-tert-butyl-N-(2-chloro-4-trifluoromethanesulfonylphenyl)-2-hydroxy-6-methylbenzamide in acetic acid (50 mL) and the mixture was stirred at room temperature for 2 h. The precipitate was isolated by filtration and washed with acetic acid and petroleum ether to give the pure title compound as yellow crystals; ¹H NMR (CDCl₃): δ 1.44 (s, 9H) 2.79 (s, 3H) 8.05 (dd, J=9.10, 2.02 Hz, 1H) 8.09 (s, 1H) 8.13 (d, J=2.02 Hz, 1H) 8.47 (br. s., 1H) 8.99 (d, J=9.10 Hz, 1H) 10.05 (s, 1H); HPLC-MS (Method B): m/z=517/519 (M+23), 495/497 (M+1); Rₜ=2.48 min.

### Example 111

### General Procedure (F)

### 3-tert-Butyl-5-chloro-2-hydroxy-6-methyl-N-(2-methyl-4-trifluoromethanesulfonylphenyl)-benzamide

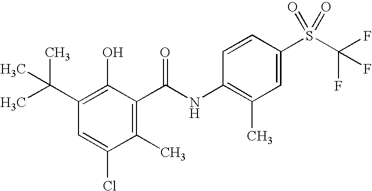

From 3-tert-butyl-5-chloro-2-hydroxy-6-methyl-benzoic acid 2-methyl-4-trifluoromethanesulfonyl aniline; ¹H NMR (CDCl₃): δ 1.40 (s, 9H) 2.44 (s, 3H) 2.59 (s, 3H) 7.41 (s, 1H) 7.60 (br. s., 1H) 7.89 (d, J=2.02 Hz, 1H) 7.89 (dd, J=8.59, 2.02 Hz, 1 Hz) 8.67 (d, J=8.59 Hz, 1H) 9.37 (br. s., 1H); HPLC-MS (Method B): m/z=486/488 (M+23), 464/466 (M+1); Rₜ=2.61 min.

### Example 112

### 3-tert-Butyl-N-(2-chloro-4-trifluoromethanesulfonyl-phenyl)-2-hydroxy-6-methyl-5-(toluene-4-sulfonylamino)-benzamide

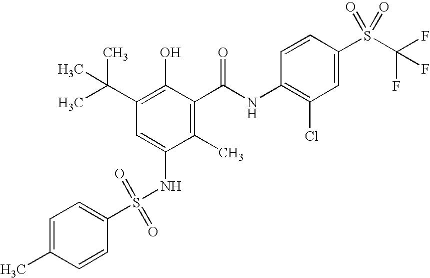

Tosyl chloride (63 mg, 0.33 mmol) was added to a solution of 3-amino-5-tert-butyl-N-(2-chloro-4-trifluoromethanesulfonyl-phenyl)-6-hydroxy-2-methyl-benzamide (139 mg, 0.3 mmol) in pyridine (1 mL) with stirring at 0° C. under nitrogen. The mixture was stirred for 2 h and evaporated to dryness. The residue was triturated with 1 M hydrochloric acid (3 mL), and the crude product was isolated by filtration and recrystallized from heptane/ethyl acetate to yield 104 mg (56%) of the pure title compound; ¹H NMR (CDCl₃): δ 1.21 (s, 9H) 2.44 (s, 3H) 2.53 (s, 3H) 6.71 (s, 1H) 7.29 (d, J=8.59 Hz, 2H) 8.01 (dd, J=8.59, 2.02 Hz, 1H) 8.10 (d, J=2.02 Hz, 1H) 8.97 (d, J=8.59 Hz, 1H) 9.97 (s, 1H); HPLC-MS (Method B): m/z=641/643 (M+23), 619/621 (M+1); Rₜ=2.51 min.

### Example 113

### General Procedure (F)

### 3-tert-Butyl-5-chloro-2-hydroxy-N-(2-methoxy-4-trifluoromethanesulfonyl-phenyl)-6-methyl-benzamide

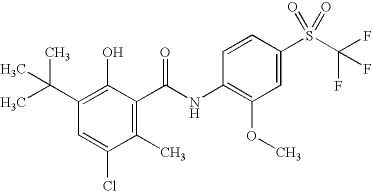

From 3-tert-butyl-5-chloro-2-hydroxy-6-methyl-benzoic acid 2-methoxy-4-trifluoromethanesulfonyl aniline; ¹H NMR (CDCl₃): δ 1.40 (s, 9H) 2.57 (s, 3H) 4.02 (s, 3H) 7.40 (s, 1H) 7.48 (s, 1H) 7.77 (dd, J=8.59, 2.02 Hz, 1H) 8.50 (br. s., 1H) 8.86 (d, J=8.59 Hz, 1H) 9.71 (s, 1H); HPLC-MS (Method B): m/z=480 (M+1); Rₜ=2.73 min.

### Example 114

### 3-Amino-5-tert-butyl-N-(2-chloro-4-trifluoromethanesulfonyl-phenyl)-6-hydroxy-2-methyl-benzamide

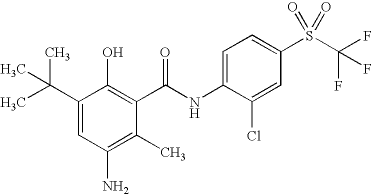

Zinc powder (6.5 g) was added in several portions over a period of 2 days to a vigorously stirred suspension of 3-tert-butyl-N-(2-chloro-4-trifluoromethanesulfonyl-phenyl)-2-hydroxy-6-methyl-5-nitro-benzamide (887 mg, 1.8 mmol) in acetic acid (50 mL). The mixture was filtered, and the filtrate was evaporated to dryness. The resulting oil was triturated with water (10 mL), and the crude product was filtered off, washed with water and dried to give 0.79 g (95%) of the pure title compound; ¹H NMR (CDCl₃): δ 1.40 (s, 9H) 2.38 (s, 3H) ca. 3.8 (very br s, 2H) 6.87 (s, 1H) 8.00 (dd, J=8.59, 2.02 Hz, 1H) 8.08 (d, J=2.02 Hz, 1H) 8.38 (br. s., 1H) ca. 8.6 (very br. s., 1H) 9.03 (d, J=8.59 Hz, 1H); HPLC-MS (Method B): m/z=465/467 (M+1), Rₜ=1.76 min.

### Example 115

### 3-Acetylamino-5-tert-butyl-N-(2-chloro-4-trifluoromethanesulfonyl-phenyl)-6-hydroxy-2-methyl-benzamide

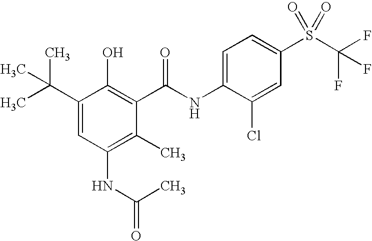

Acetic anhydride (22 μL, 0.23 mmol) was added to a solution of 3-amino-5-tert-butyl-N-(2-chloro-4-trifluoromethanesulfonyl-phenyl)-6-hydroxy-2-methyl-benzamide in acetic acid (2 mL). The vessel was sealed and the solution was heated at 150° C. for 30 min using microwave irradiation. The cooled solution was poured into water (25 mL), and the resulting precipitate was filtered off and washed with water and petroleum ether to give the crude product, which was purified by column chromatography to give 52 mg (47%) of the pure title compound; ¹H NMR (CDCl₃): δ 1.40 (s, 9H) 2.23 (s, 3H) 2.47 (s, 3H) 6.97 (br. s., 1H) 7.30 (s, 1H) 8.01 (dd, J=8.59, 2.02 Hz, 1H) 8.09 (d, J=2.02 Hz, 1H) 8.54 (br. s., 1H) 9.00 (d, J=8.59 Hz, 1H) 9.75 (br. s., 1H); HPLC-MS (Method B): m/z=507/509 (M+1); Rₜ=2.22 min.

### Example 116

### General Procedure (A)

### 1-Hydroxy-4-(4-methoxyphenyl)naphthalene-2-carboxylic acid (2-methyl-4-trifluoromethanesulfonylphenyl)amide

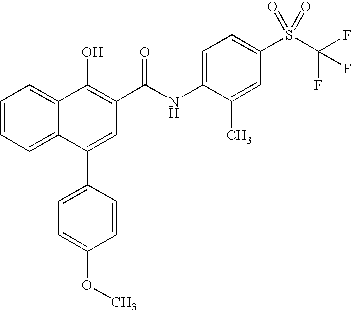

Step A: from 4-bromo-1-hydroxy-naphthalene-2-carboxylic acid and 4-methoxyphenyl-boronic acid.

Step B: from the product formed in step A and 2-methyl-4-trifluoromethanesulfonyl aniline

¹H NMR (DMSO-d₆): δ 2.49 (s, 3H) 3.91 (s, 3H) 7.06 (d, J=8.59 Hz, 2H) 7.29 (s, 1H) 7.04 (d, J=8.59 Hz, 2H) 7.61 (m, 2H) 7.81 (m, 1H) 7.90 (d, J=2.02 Hz, 1H) 7.96 (dd, J=2.02 and 8.59 Hz, 1H) 8.12 (br.s, 1H) 8.55 (m, 2H) 13.10 (br.s, 1H); HPLC-MS (Method B): m/z=516 (M+1); Rₜ=2.845 min.

### Example 117

### General Procedure (A)

### 4-(4-Fluorophenyl)-1-hydroxynaphthalene-2-carboxylic acid (4-trifluoromethanesulfonylphenyl)amide

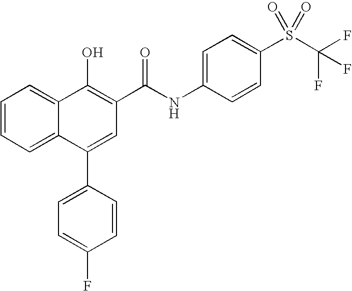

Step A: from 4-bromo-1-hydroxy-naphthalene-2-carboxylic acid and 4-fluorophenylboronic acid.

Step B: from the product formed in step A and 4-trifluoromethanesulfonyl aniline.

¹H NMR (DMSO-d₆): δ 7.40 (dd, J=8.34 and 8.34 Hz, 2H) 7.57 (dd, J=7.58 and 7.58 Hz, 2H) 7.64-7.74 (m, 3H) 8.10 (s, 1H) 8.18 (d, J=8.59 Hz, 2H) 8.25 (d, J=8.59 Hz, 2H) 8.45 (d, J=8.08 Hz, 1H) 11.09 (br.s, 1H) 13.49 (br.s, 1H); HPLC-MS (Method B): m/z=490 (M+1); Rₜ=2.937 min.

### Example 118

### General Procedure (A)

### 4-(4-Fluorophenyl)-1-hydroxynaphthalene-2-carboxylic acid (2-methyl-4-trifluoromethanesulfonylphenyl)amide

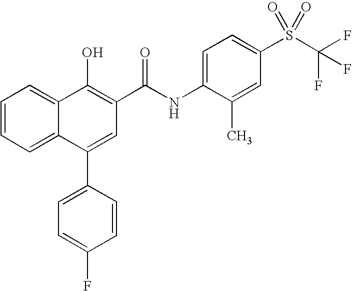

Step A: from 4-bromo-1-hydroxy-naphthalene-2-carboxylic acid and 4-fluorophenylboronic acid.

Step B: from the product formed in step A and 2-methyl-4-trifluoromethanesulfonyl aniline.

¹H NMR (DMSO-d₆): δ 2.48 (s, 3H) 7.40 (dd, J=8.84 and 8.84 Hz, 2H) 7.57 (dd, J=8.59 and 8.59 Hz, 2H) 7.64-7.71 (m, 2H) 7.77 (d, J=7.58 Hz, 1H) 8.04-8.12 (m, 4H) 8.45 (d, J=7.58 Hz, 1H) 10.93 (br.s, 1H); HPLC-MS (Method B): m/z=504 (M+1); Rₜ=2.876 min.

### Example 119

### General Procedure (F)

### 1-Hydroxynaphthalene-2-carboxylic acid (2-chloro-4-trifluoromethanesulfonylphenyl)amide

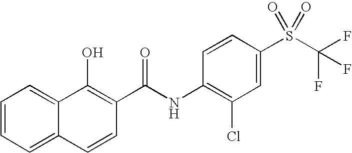

From 1-hydroxy-naphthalene-2-carboxylic acid and 2-chloro-4-trifluoromethanesulfonyl aniline.

¹H NMR (DMSO-d₆): δ 7.57 (d, J=9.1 Hz, 1H) 7.63 (dd, J=8.59 and 8.59 Hz, 1H) 7.70 (dd, J=8.59 and 8.59 Hz, 1H) 7.96 (d, J=8.08 Hz, 1H) 8.09 (d, J=9.09 Hz, 1H) 8.21 (dd, J=2.27 and 8.84 Hz, 1H) 8.36 (d, J=2.53 Hz, 1H) 8.40 (d, J=8.59 Hz, 1H) 8.77 (d, J=8.59 Hz, 1H) 11.54 (br.s, 1H); HPLC-MS (Method B): m/z=430 (M+1); Rₜ=1.524 min.

### Example 120

### 1-Hydroxy-8-methylnaphthalene-2-carboxylic acid (2-chloro-4-trifluoromethanesulfonylphenyl)amide

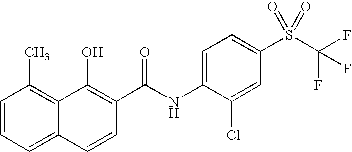

8-Methyl-naphthalen-1-ol (142 mg, 0.898 mmol) (W. Cocker et al., J. Chem. Soc. 1960, 2230) was dissolved in 5 ml of dry toluene under a nitrogen atmosphere, and the solution was cooled to 0° C. Boron trichloride (1M in dichloromethane) (0.98 ml, 0.98 mmol) was added and the reaction mixture was stirred for 10 min at 0° C. 2-Chloro-1-isocyanato-4-trifluoromethanesulfonyl-benzene (256.3 mg, 0.898 mmol) was dissolved in 10 ml dry toluene and added to the reaction mixture. After 5 min at 0° C. the mixture was heated at 120° C. for 1.5 h. The reaction mixture was suspended in methanol and filtered, and the isolated product was then recrystallized from 15 ml of acetonitrile to afford 118 mg (30%) of the title compound as a yellow solid.

¹H NMR (CDCl₃): δ 3.01 (s, 3H) 7.32 (m, 2H) 7.44 (d, J=9.1, 1H) 7.49 (d, J=7.58 and 7.58 Hz, 1H) 7.62 (d, J=8.08 Hz, 1H) 8.02 (dd, J=2.02 and 8.59 Hz, 1H) 8.13 (d, J=2.02 Hz, 1H) 8.95 (d, J=9.1 Hz, 1H) 13.49 (br. s, 1H); HPLC-MS (Method B): m/z=444 (M+1); Rₜ=2.82 min.

### Example 121

### General Procedure (F)

### N-(3-Chloro-5-trifluoromethanesulfonyl-phenyl)-2-hydroxy-benzamide

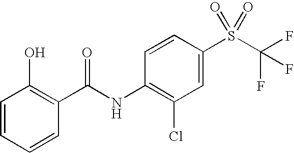

From salicylic acid and 2-chloro-4-trifluoromethanesulfonyl aniline.

¹H NMR (DMSO-d₆): δ 6.98-7.14 (m, 2H) 7.44-7.57 (m, 1H) 8.06 (dd, J=7.91, 1.88 Hz, 1H) 8.18 (dd, J=9.04, 2.26 Hz, 1H) 8.31 (d, J=2.26 Hz, 1H) 9.05 (d, J=8.67 Hz, 1H) 11.59 (br. s., 1H) 12.26 (br. s., 1H).

### Example 122

### General Procedure (H)

### 3-Benzylsulfanyl-5-tert-butyl-N-(2-chloro-4-trifluoromethanesulfonyl-phenyl)-6-hydroxy-2-methyl-benzamide

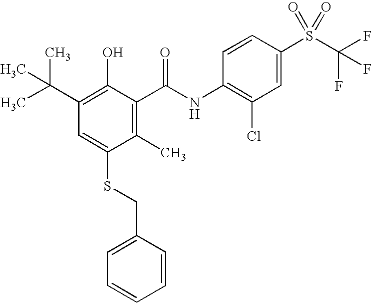

Step A: from 5-tert-butyl-6-hydroxy-2-methyl-benzoic acid and disulfur dichloride.

Step B: from the product formed in step A, benzyl bromide and sodium borohydride.

Step C: from the product formed in step B and 2-chloro-4-trifluoromethanesulfonyl aniline.

¹H NMR (DMSO-d₆): δ ppm 1.26 (s, 9H) 2.34 (s, 3H) 3.99 (s, 2H) 7.05 (s, 1H) 7.18-7.34 (m, 5H) 8.17 (d, 1H) 8.24 (d, 1H) 8.59 (dd, 1H) 9.04 (s, 1H) 10.54 (br. s., 1H); HPLC-MS (Method B): m/z=572, 574 (M+1); Rₜ=2.97 min.

### Example 123

### General Procedure (H)

### 3-tert-Butyl-N-(2-chloro-4-trifluoromethanesulfonyl-phenyl)-2-hydroxy-5-isopropylsulfanyl-6-methyl-benzamide

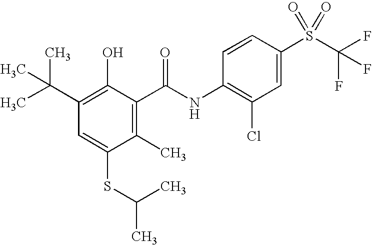

Step A: from 5-tert-butyl-6-hydroxy-2-methyl-benzoic acid and disulfur dichloride.

Step B: from the product formed in step A, 2-iodopropane and sodium borohydride.

Step C: from the product formed in step B and 2-chloro-4-trifluoromethanesulfonyl aniline.

¹H NMR (DMSO-d₆): δ ppm 1.21 (d, J=7.07 Hz, 6H) 1.36 (s, 9H) 2.38 (s, 3H) 3.09-3.24 (m, 1H) 7.34 (s, 1H) 8.18 (d, 1H) 8.24 (s, 1H) 8.59 (d, 1H) 9.12 (s, 1H) 10.64 (br. s., 1H); HPLC-MS (Method B): m/z=524, 526 (M+1); Rₜ=2.96 min.

### Example 124

### General Procedure (H)

### 3-tert-Butyl-N-(2-chloro-4-trifluoromethanesulfonyl-phenyl)-2-hydroxy-6-methyl-5-propylsulfanyl-benzamide

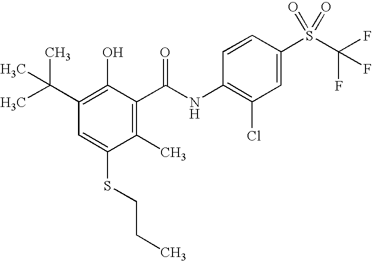

Step A: from 5-tert-butyl-6-hydroxy-2-methyl-benzoic acid and disulfur dichloride.

Step B: from the product formed in step A, 1-iodopropane and sodium borohydride.

Step C: from the product formed in step B and 2-chloro-4-trifluoromethanesulfonyl aniline.

¹H NMR (DMSO-d₆): δ ppm 0.97 (t, J=7.35 Hz, 3H) 1.37 (s, 9H) 1.47-1.62 (m, 2H) 2.34 (s, 3H) 2.78 (t, J=7.16 Hz, 2H) 7.30 (s, 1H) 8.19 (d, J=9.42 Hz, 1H) 8.24 (s, 1H) 8.59 (d, J=8.67 Hz, 1H) 9.01 (s, 1H) 10.61 (br. s., 1H); HPLC-MS (Method B): m/z=524, 526 (M+1); Rₜ=2.93 min.

### Example 125

### General Procedure (H)

### 3-tert-Butyl-N-(2-chloro-4-trifluoromethanesulfonyl-phenyl)-2-hydroxy-6-methyl-5-(propane-1-sulfinyl)-benzamide

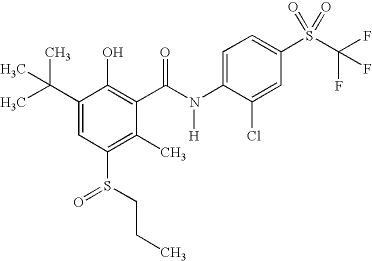

Step A: from 5-tert-butyl-6-hydroxy-2-methyl-benzoic acid and disulfur dichloride.

Step B: from the product formed in step A, 1-iodopropane and sodium borohydride.

Step C: from the product formed in step B and 2-chloro-4-trifluoromethanesulfonyl aniline.

Step D: from the product formed in step C and 3-chloroperoxybenzoic acid (1.0 equivalent), in dichloromethane at room temperature.

¹H NMR (DMSO-d₆): δ ppm 1.01 (t, J=7.35 Hz, 3H) 1.40 (s, 9H) 1.51-1.79 (m, 2H) 2.27 (s, 3H) 2.58-2.70 (m, 1H) 2.71-2.89 (m, 1H) 7.68 (s, 1H) 8.18 (d, 1H) 8.25 (s, 1H) 8.62 (d, 1H) 9.59 (s, 1H) 10.72 (br. s., 1H); HPLC-MS (Method B): m/z=541, 543 (M+1); Rₜ=2.33 min.

### Example 126

### General Procedure (H)

### 3-tert-Butyl-N-(2-chloro-4-trifluoromethanesulfonyl-phenyl)-2-hydroxy-6-methyl-5-phenylmethanesulfinyl-benzamide

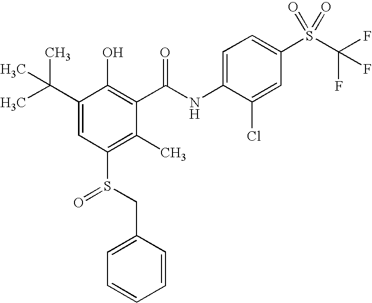

Step A: from 5-tert-butyl-6-hydroxy-2-methyl-benzoic acid and disulfur dichloride.

Step B: from the product formed in step A, benzyl bromide and sodium borohydride.

Step C: from the product formed in step B and 2-chloro-4-trifluoromethanesulfonyl aniline.

Step D: from the product formed in step C and hydrogen peroxide in acetic acid at room temperature.

¹H NMR (DMSO-d₆): δ ppm 1.26 (s, 9H) 2.24 (s, 3H) 3.91 (d, J=13.14 Hz, 1H) 4.19 (d, J=12.13 Hz, 1H) 7.00 (d, J=7.07 Hz, 2H) 7.15 (s, 1H) 7.21-7.34 (m, 3H) 8.20 (d, 1H) 8.26 (s, 1H) 8.61 (d, 1H) 9.55 (s, 1H) 10.61 (br. s., 1H); HPLC-MS (Method B): m/z=588, 590 (M+1); Rₜ=2.53 min.

### Example 127

### General Procedure (F)

### 3-tert-Butyl-5-chloro-2-hydroxy-6-methyl-N-(2-propyl-4-trifluoromethanesulfonylphenyl)-benzamide

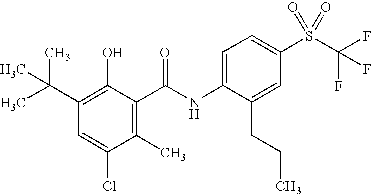

From 3-chloro-5-tert-butyl-6-hydroxy-2-methyl-benzoic acid and 2-propyl-4-trifluoromethanesulfonyl aniline.

¹H NMR (DMSO-d₆): δ 0.91 (t, J=7.33 Hz, 3H) 1.37 (s, 9H) 1.53-1.65 (m, 2H) 2.27 (s, 3H) 2.76-2.86 (m, 2H) 7.23 (s, 1H) 7.93 (s, 1H) 8.02 (d, J=8.08 Hz, 1H) 8.42 (d, J=8.08 Hz, 1H) 9.03 (s, 1H) 10.32 (br. s., 1H); HPLC-MS (Method B): m/z=492, 494 (M+1); Rₜ=2.81 min.

### Example 128

### General Procedure (H)

### 3-tert-Butyl-N-(2-chloro-4-trifluoromethanesulfonyl-phenyl)-2-hydroxy-5-isobutylsulfanyl-6-methyl-benzamide

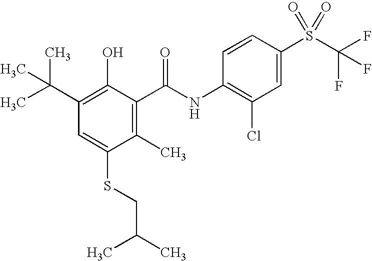

Step A: from 5-tert-butyl-6-hydroxy-2-methyl-benzoic acid and disulfur dichloride.

Step B: from the product formed in step A, 1-iodo-2-methylpropane and sodium borohydride.

Step C: from the product formed in step B and 2-chloro-4-trifluoromethanesulfonyl aniline.

¹H NMR (DMSO-d₆): δ ppm 0.99 (d, J=6.78 Hz, 6H) 1.37 (s, 9H) 1.66-1.80 (m, 1H) 2.34 (s, 3H) 2.69 (d, J=6.41 Hz, 2H) 7.29 (s, 1H) 8.19 (d, J=8.67 Hz, 1H) 8.24 (s, 1H) 8.59 (d, J=8.29 Hz, 1H) 9.00 (s, 1H) 10.60 (br. s., 1H); HPLC-MS (Method B): m/z=538, 540 (M+1); Rₜ=2.94 min.

### Example 129

### General Procedure (F)

### 3-tert-Butyl-5-chloro-N-(2-ethyl-4-trifluoromethanesulfonyl-phenyl)-2-hydroxy-6-methyl-benzamide

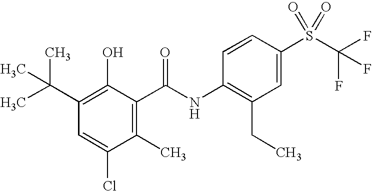

From 3-chloro-5-tert-butyl-6-hydroxy-2-methyl-benzoic acid and 2-ethyl-4-trifluoromethanesulfonyl aniline.

¹H NMR (DMSO-d₆): δ ppm 1.19 (t, J=7.35 Hz, 3H) 1.37 (s, 9H) 2.27 (s, 3H) 2.84 (q, J=7.28 Hz, 2H) 7.23 (s, 1H) 7.93 (s, 1H) 8.03 (d, J=9.04 Hz, 1H) 8.40 (d, J=8.67 Hz, 1H) 9.04 (s, 1H) 10.37 (br. s., 1H); HPLC-MS (Method B): m/z=478, 480 (M+1); Rₜ=2.67 min.

### Example 130

### General Procedure (H)

### 3-tert-Butyl-N-(2-chloro-4-trifluoromethanesulfonyl-phenyl)-2-hydroxy-6-methyl-5-phenylmethanesulfonyl-benzamide

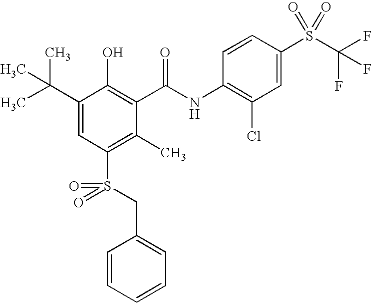

Step A: from 5-tert-butyl-6-hydroxy-2-methyl-benzoic acid and disulfur dichloride.

Step B: from the product formed in step A, benzylbromide and sodium borohydride.

Step C: from the product formed in step B and 2-chloro-4-trifluoromethanesulfonyl aniline.

Step E: from the product formed in step C and hydrogen peroxide in acetic acid at 100° C.

¹H NMR (DMSO-d₆): δ d ppm 1.25 (s, 9H) 2.50 (s, 3H) 4.54 (s, 2H) 7.14 (d, J=6.57 Hz, 2H) 7.26-7.40 (m, 4H) 8.22 (d, 1H) 8.26 (s, 1H) 8.64 (d, 1H) 10.08 (s, 1H) 10.81 (br. s., 1H); HPLC-MS (Method B): m/z=604, 606 (M+1); Rₜ=2.53 min.

### Example 131

### General Procedure (H)

### 3-tert-Butyl-N-(2-chloro-4-trifluoromethanesulfonyl-phenyl)-2-hydroxy-6-methyl-5-(propane-1-sulfonyl)-benzamide

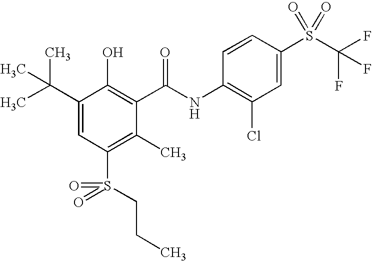

Step A: from 5-tert-butyl-6-hydroxy-2-methyl-benzoic acid and disulfur dichloride.

Step B: from the product formed in step A, 1-iodopropane and sodium borohydride.

Step C: from the product formed in step B and 2-chloro-4-trifluoromethanesulfonyl aniline.

Step E: from the product formed in step C and hydrogen peroxide in acetic acid at 100° C.

¹H NMR (DMSO-d₆): δ 0.96 (t, J=7.35 Hz, 3H) 1.39 (s, 9H) 1.54-1.71 (m, 2H) 2.52 (s, 3H) 3.14-3.27 (m, 2H) 7.81 (s, 1H) 8.22 (d, J=8.67 Hz, 1H) 8.26 (s, 1H) 8.65 (d, J=8.67 Hz, 1H) 10.11 (s, 1H) 10.86 (br. s., 1H); HPLC-MS (Method B): m/z=556, 558 (M+1); Rₜ=2.48 min.

### Example 132

### General Procedure (G)

### 5-tert-Butyl-4′-cyano-4-hydroxy-2-methyl-biphenyl-3-carboxylic acid (2-methyl-4-trifluoromethanesulfonyl-phenyl)-amide

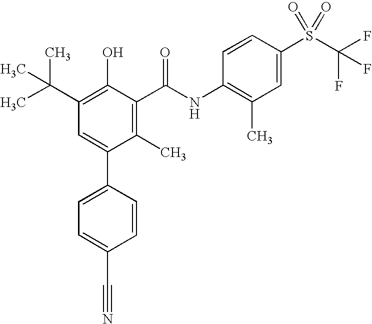

Step A: from 3-iodo-5-tert-butyl-6-hydroxy-2-methyl-benzoic acid and 4-cyano-phenyl-boronic acid.

Step B: from the product formed in step A and 2-methyl-4-trifluoromethanesulfonyl aniline.

¹H NMR (DMSO-d₆): δ ppm 1.40 (s, 9H) 2.18 (s, 3H) 2.46 (s, 3H) 7.10 (s, 1H) 7.53 (d, J=8.59 Hz, 2H) 7.91 (d, J=8.08 Hz, 2H) 8.01 (d, 1H) 8.00 (s, 1H) 8.39 (d, J=8.08 Hz, 1H) 9.11 (s, 1H) 10.39 (br. s., 1H); HPLC-MS (Method B): m/z=531 (M+1); Rₜ=2.79 min.

### Example 133

### General Procedure (G)

### 5-tert-Butyl-4′-cyano-4-hydroxy-2-methyl-biphenyl-3-carboxylic acid (2-ethyl-4-trifluoromethanesulfonyl-phenyl)-amide

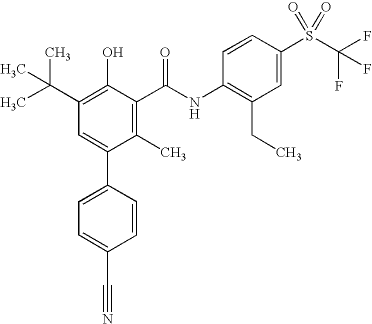

Step A: from 3-iodo-5-tert-butyl-6-hydroxy-2-methyl-benzoic acid and 4-cyano-phenyl-boronic acid.

Step B: from the product formed in step A and 2-ethyl-4-trifluoromethanesulfonyl aniline.

¹H NMR (DMSO-d₆): δ ppm 1.09 (t, J=6.82 Hz, 3H) 1.40 (s, 9H) 2.18 (s, 3H) 2.87 (q, J=7.58 Hz, 2H) 7.10 (s, 1H) 7.54 (d, J=8.08 Hz, 2H) 7.90 (d, 2H) 7.91 (s, 1H) 8.02 (d, J=8.59 Hz, 1H) 8.44 (d, J=8.59 Hz, 1H) 9.05 (s, 1H) 10.41 (br. s., 1H); HPLC-MS (Method B): m/z=545 (M+1); Rₜ=2.77 min.

### Example 134

### General Procedure (G)

### 5-tert-Butyl-4′-cyano-4-hydroxy-2-methyl-biphenyl-3-carboxylic acid (2-cyano-4-trifluoromethanesulfonyl-phenyl)-amide

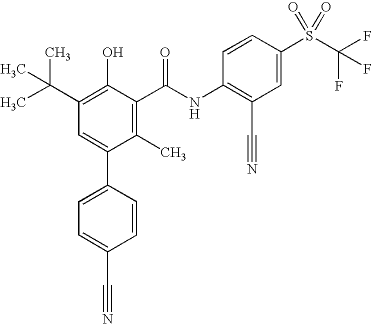

Step A: from 3-iodo-5-tert-butyl-6-hydroxy-2-methyl-benzoic acid and 4-cyano-phenyl-boronic acid.

Step B: from the product formed in step A and 2-cyano-4-trifluoromethanesulfonyl aniline.

¹H NMR (DMSO-d₆): δ ppm 1.38 (s, 9H) 2.19 (s, 3H) 7.13 (s, 1H) 7.53 (d, J=8.08 Hz, 2H) 7.91 (d, J=8.08 Hz, 2H) 8.41 (d, 1H) 8.47 (d, 1H) 8.71 (s, 1H) 9.26 (s, 1H) 11.50 (br. s., 1H); HPLC-MS (Method B): m/z=542 (M+1); Rₜ=2.62 min.

### Example 135

### General Procedure (H)

### 3-tert-Butyl-N-(2-chloro-4-trifluoromethanesulfonyl-phenyl)-2-hydroxy-6-methyl-5-(propane-2-sulfonyl)-benzamide

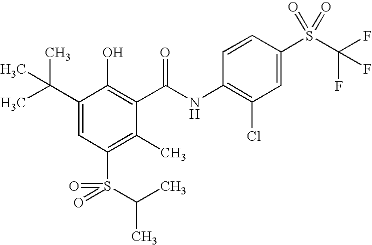

Step A: from 5-tert-butyl-6-hydroxy-2-methyl-benzoic acid and disulfur dichloride.

Step B: from the product formed in step A, 2-iodopropane and sodium borohydride.

Step C: from the product formed in step B and 2-chloro-4-trifluoromethanesulfonyl aniline.

Step E: from the product formed in step C and hydrogen peroxide in acetic acid at 100° C.

¹H NMR (DMSO-d₆): δ ppm 1.18 (d, J=7.07 Hz, 6H) 1.39 (s, 9H) 2.28 (s, 3H) 3.36-3.43 (m, 1H) 7.77 (s, 1H) 8.22 (d, J=8.59 Hz, 1H) 8.26 (s, 1H) 8.65 (d, J=9.10 Hz, 1H) 10.14 (s, 1H) 10.87 (br. s., 1H); HPLC-MS (Method B): m/z=556, 558 (M+1); Rₜ=2.40 min.

### Example 136

### General Procedure (H)

### 3-tert-Butyl-N-(2-chloro-4-trifluoromethanesulfonyl-phenyl)-2-hydroxy-6-methyl-5-(propane-2-sulfinyl)-benzamide

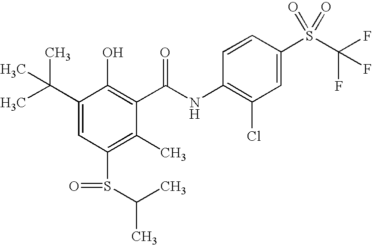

Step A: from 5-tert-butyl-6-hydroxy-2-methyl-benzoic acid and disulfur dichloride.

Step B: from the product formed in step A, 2-iodopropane and sodium borohydride.

Step C: from the product formed in step B and 2-chloro-4-trifluoromethanesulfonyl aniline.

Step D: from the product formed in step C and hydrogen peroxide in acetic acid at room temperature.

¹H NMR (DMSO-d₆): δ ppm 1.01 (d, J=6.57 Hz, 3H) 1.21 (d, J=7.07 Hz, 3H) 1.40 (s, 9H) 2.28 (s, 3H) 2.77-2.91 (m, 1H) 7.60 (s, 1H) 8.20 (d, J=9.10 Hz, 1H) 8.25 (s, 1H) 8.63 (d, J=8.59 Hz, 1H) 9.61 (s, 1H) 10.74 (br. s., 1H); HPLC-MS (Method B): m/z=540, 542 (M+1); Rₜ=2.45 min.

### Example 137

### General Procedure (G)

### 5-tert-Butyl-4′-cyano-4-hydroxy-2-methyl-biphenyl-3-carboxylic acid (2-propyl-4-trifluoromethanesulfonyl-phenyl)-amide

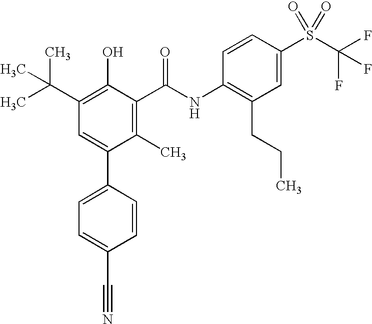

Step A: from 3-iodo-5-tert-butyl-6-hydroxy-2-methyl-benzoic acid and 4-cyano-phenyl-boronic acid.

Step B: from the product formed in step A and 2-propyl-4-trifluoromethanesulfonyl aniline.

¹H NMR (CDCl₃): δ ppm 1.03 (t, J=7.33 Hz, 3H) 1.43 (s, 9H) 1.65-1.79 (m, 2H) 2.39 (s, 3H) 2.68 (t, J=7.83 Hz, 2H) 7.25 (d, 2H) 7.42 (d, J=8.59 Hz, 2H) 7.73 (s, 1H) 7.75 (s, 1H) 7.89 (s, 1H) 7.97 (dd, J=8.84, 2.27 Hz, 1H) 8.62 (d, J=8.59 Hz, 1H) 9.34 (s, 1H); HPLC-MS (Method B): m/z=559 (M+1); Rₜ=2.83 min.

### Example 138

### General Procedure (G)

### 3-tert-Butyl-5-chloro-N-(3-cyano-4-trifluoromethanesulfonyl-phenyl)-2-hydroxy-6-methyl-benzamide

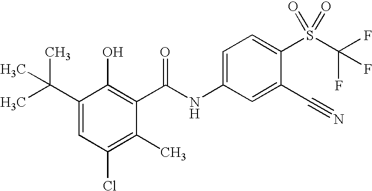

Step B: from 3-chloro-5-tert-butyl-6-hydroxy-2-methyl-benzoic acid and 3-cyano-4-trifluoromethanesulfonyl aniline.

¹H NMR (DMSO-d₆): δ ppm 1.36 (s, 9H) 2.22 (s, 3H) 7.26 (s, 1H) 8.32 (dd, 1H) 8.39 (d, 1H) 8.58 (d, J=2.02 Hz, 1H) 9.30 (s, 1H) 11.59 (br. s., 1H); HPLC-MS (Method B): m/z=475, 477 (M+1); Rₜ=2.58 min.

### Example 139

### General Procedure (H)

### 3-tert-Butyl-N-(2-chloro-4-trifluoromethanesulfonyl-phenyl)-2-hydroxy-6-methyl-5-(2-methyl-propane-1-sulfinyl)-benzamide

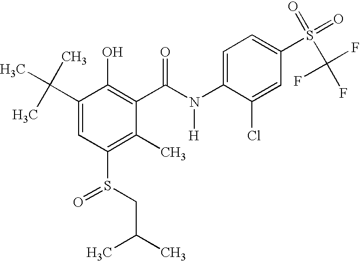

Step A: from 5-tert-butyl-6-hydroxy-2-methyl-benzoic acid and disulfur dichloride.

Step B: from the product formed in step A, 1-iodo-2-methylpropane and sodium borohydride.

Step C: from the product formed in step B and 2-chloro-4-trifluoromethanesulfonyl aniline.

Step D: from the product formed in step C and 3-chloroperoxybenzoic acid (1.0 equivalent) in dichloromethane at room temperature.

¹H NMR (DMSO-d₆): δ ppm 1.02 (d, J=6.41 Hz, 3H) 1.11 (d, J=6.78 Hz, 3H) 2.05-2.19 (m, 1H) 2.26 (s, 3H) 2.56-2.63 (m, 2H) 7.71 (s, 1H) 8.22 (d, 1H) 8.25 (s, 1H) 8.61 (d, 1H) 9.58 (s, 1H) 10.69 (br. s., 1H); HPLC-MS (Method B): m/z=554, 556 (M+1); Rₜ=2.41 min.

### Example 140

### General Procedure (F)

### N-(2-Chloro-4-trifluoromethanesulfonylphenyl)-2-hydroxy-3-isopropyl-6-methylbenzamide

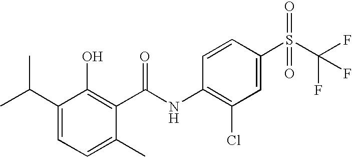

From 2-hydroxy-3-isopropyl-6-methylbenzoic acid and 2-chloro-4-(trifluoromethanesulfonyl)aniline; ¹H NMR (400 MHz, DMSO-d₆): δ 1.16 (d, J=6.57 Hz, 6H), 2.28 (s, 3H), 3.28 (m, J=6.6 Hz, 1H), 6.78 (d, J=8.08 Hz, 1H), 7.16 (d, J=8.08 Hz, 1H), 8.17 (d, J=8.59 Hz, 1H), 8.25 (s, 1H), 8.54 (d, J=8.59 Hz, 1H), 8.95 (s, 1H), 10.44 (s, 1H) ppm; HPLC-MS (Method B): m/z=436/438 (M+H), Rₜ=2.66 min.

### Example 141

### General Procedure (F)

### N-(2-Chloro-4-trifluoromethanesulfonylphenyl)-2-hydroxy-6-methylbenzamide

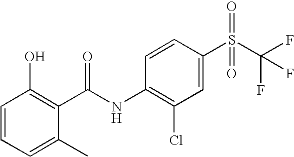

From 2-hydroxy-6-methylbenzoic acid and 2-chloro-4-(trifluoromethanesulfonyl)aniline; ¹H NMR (400 MHz, DMSO-d₆): δ 2.34 (s, 3H), 6.76 (d, J=7.58 Hz, 1H), 6.80 (d, J=8.08 Hz, 1H), 7.20 (t, J=7.83 Hz, 1H), 8.16 (dd, J=8.59, 2.02 Hz, 1H), 8.26 (d, J=2.02 Hz, 1H), 8.62 (d, J=8.59 Hz, 1H), 10.35 (s, 1H), 10.46 (s, 1H) ppm; HPLC-MS (Method B): m/z=394/396 (M+H), Rₜ=2.30 min.

### Example 142

### General Procedure (F)

### 5-Chloro-N-(2-chloro-4-trifluoromethanesulfonylphenyl)-2-hydroxybenzamide

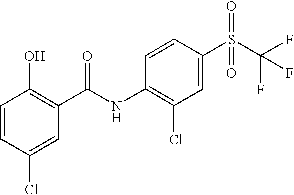

From 5-chloro-2-hydroxybenzoic acid and 2-chloro-4-(trifluoromethanesulfonyl)aniline; ¹H NMR (300 MHz, DMSO-d₆): δ 7.11 (d, J=8.67 Hz, 1H), 7.56 (dd, J=8.85, 2.83 Hz, 1H), 7.98 (d, J=3.01 Hz, 1H), 8.19 (dd, J=8.85, 2.07 Hz, 1H), 8.32 (d, J=2.26 Hz, 1H), 9.01 (d, J=9.04 Hz, 1H), 11.52 (s, 1H), 12.63 (s(br), 1H) ppm; HPLC-MS (Method B): m/z=414/416/418 (M+H), Rₜ=2.48 min.

### Example 143

### General Procedure (F)

### N-(2-Chloro-4-trifluoromethanesulfonylphenyl)-2-hydroxy-6-isopropyl-3-methylbenzamide

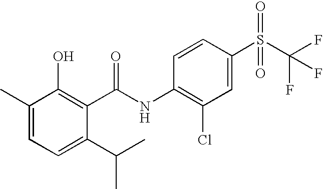

From 2-hydroxy-6-isopropyl-3-methylbenzoic acid and 2-chloro-4-(trifluoromethanesulfonyl)aniline; ¹H NMR (300 MHz, DMSO-d₆): δ 1.19 (d, J=6.78 Hz, 6H), 2.18 (s, 3H), 2.97 (m, J=6.8 Hz, 1H), 6.81 (d, J=7.91 Hz, 1H), 7.14 (d, J=7.54 Hz, 1H), 8.16 (dd, J=8.29, 2.26 Hz, 1H), 8.24 (d, J=2.26 Hz, 1H), 8.50 (d, J=8.29 Hz, 1H), 8.84 (s(br), 1H), 10.55 (s(br), 1H) ppm; HPLC-MS (Method B): m/z=436/438 (M+H), Rₜ=2.50 min.

### Example 144

### General Procedure (F)

### N-(2-Chloro-4-trifluoromethanesulfonylphenyl)-5-fluoro-2-hydroxybenzamide

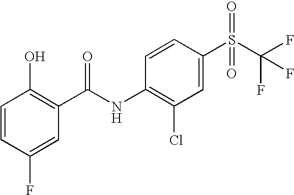

From 5-fluoro-2-hydroxybenzoic acid and 2-chloro-4-(trifluoromethanesulfonyl)aniline; ¹H NMR (300 MHz, DMSO-d₆): δ 7.10 (dd, J=9.04, 4.52 Hz, 1H), 7.40 (dt, J=8.38, 3.20 Hz, 1H), 7.74 (dd, J=9.42, 3.39 Hz, 1H), 8.19 (dd, J=8.85, 2.07 Hz, 1H), 8.32 (d, J=2.26 Hz, 1H), 9.02 (d, J=9.04 Hz, 1H), 11.59 (s(br), 1H), 12.35 (s(br), 1H) ppm; HPLC-MS (Method B): m/z=398/400 (M+H), Rₜ=2.22 min.

### Example 145

### General Procedure (F)

### 3-Chloro-N-(2-chloro-4-trifluoromethanesulfonylphenyl)-6-hydroxy-5-isopropyl-2-methylbenzamide

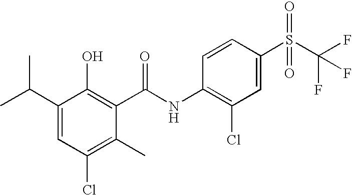

From 3-chloro-6-hydroxy-5-isopropyl-2-methylbenzoic acid (obtainable by chlorination of 2-hydroxy-3-isopropyl-6-methylbenzoic acid by the procedure described in Ind. J. Chem., Sect B 2004, 43, 1335-1338) and 2-chloro-4-(trifluoromethanesulfonyl)aniline; ¹H NMR (300 MHz, DMSO-d₆): δ1.17 (d, J=6.78 Hz, 6H), 2.25 (s, 3H), 3.28 (m, J=6.8 Hz, 1H), 7.26 (s, 1H), 8.19 (d, J=8.67 Hz, 1H), 8.26 (d, J=1.88 Hz, 1H), 8.53 (d, J=8.67 Hz, 1H), 9.13 (s, 1H), 10.61 (s, 1H) ppm; HPLC-MS (Method B): m/z=470/472/474 (M+H), Rₜ=2.56 min.

### Example 146

### General Procedure (E)

### 3-Chloro-N-(2-chloro-4-trifluoromethanesulfonylphenyl)-6-hydroxy-2-methylbenzamide

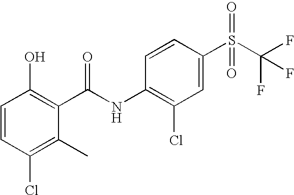

From 3-chloro-6-hydroxy-2-methylbenzoic acid (obtainable by chlorination of 2-hydroxy-6-methylbenzoic acid by the procedure described in Ind. J. Chem., Sect B 2004, 43, 1335-1338) and 2-chloro-4-(trifluoromethanesulfonyl)aniline; ¹H NMR (300 MHz, DMSO-d₆): δ 2.29 (s, 3H), 6.80 (d, J=8.67 Hz, 1H), 7.33 (d, J=8.67 Hz, 1H), 8.17 (d, J=8.67 Hz, 1H), 8.26 (d, J=1.88 Hz, 1H), 8.51 (d, J=8.67 Hz, 1H), 10.23 (s(br), 1H), 10.58 (s(br), 1H) ppm; HPLC-MS (Method B): m/z=428/430/432 (M+H), Rₜ=2.24 min.

### Example 147

### General Procedure (E)

### 3,5-Dichloro-N-(2-chloro-4-trifluoromethanesulfonylphenyl)-2-hydroxy-6-methylbenzamide

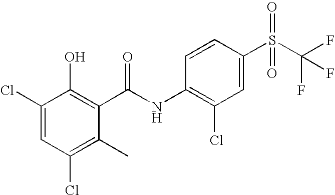

From 3,5-dichloro-2-hydroxy-6-methylbenzoic acid (obtainable by dichlorination of 2-hydroxy-6-methylbenzoic acid by the procedure described in Ind. J. Chem., Sect B 2004, 43, 1335-1338) and 2-chloro-4-(trifluoromethanesulfonyl)aniline; ¹H NMR (300 MHz, CDCl₃): δ 2.42 (s, 3H), 6.65 (s, 1H), 7.46 (s, 1H), 7.94 (d, J=9.04 Hz, 1H), 8.02 (s, 1H), 8.42 (s, 1H), 8.92 (d, J=8.67 Hz, 1H) ppm; HPLC-MS (Method B): m/z=462/464/466 (M+H), Rₜ=2.26 min.

### Example 148

### 3-Chloro-N-(2-chloro-4-trifluoromethanesulfonylphenyl)-6-hydroxy-2-isopropyl-5-methylbenzamide

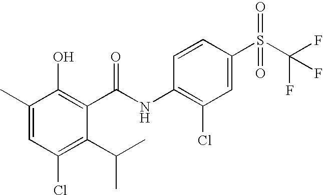

Obtainable by chlorination of N-(2-chloro-4-trifluoromethanesulfonylphenyl)-2-hydroxy-6-isopropyl-3-methylbenzamide by the procedure described in Ind. J. Chem., Sect. B 2004, 43, 1335-1338; ¹H NMR (400 MHz, DMSO-d₆): δ 1.34 (d, J=7.58 Hz, 6H), 2.18 (s, 3H), 3.17 (m, 1H), 7.22 (s, 1H), 8.18 (dd, J=8.59, 2.02 Hz, 1H), 8.25 (d, J=2.02 Hz, 1H), 8.49 (d, J=8.59 Hz, 1H), 9.10 (s, 1H), 10.61 (s, 1H) ppm; HPLC-MS (Method B): m/z=470/472 (M+H), Rₜ=2.58 min.

### Example 149

### General Procedure (C)

### N-(2-Chloro-4-trifluoromethanesulfonylphenyl)-2-hydroxy-3,4,6-trimethylbenzamide

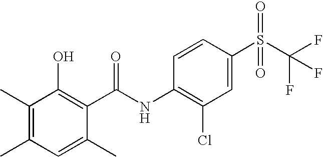

From 2,3,5-trimethylphenol and 2-chloro-1-isocyanato-4-(trifluoromethanesulfonyl)benzene; ¹H NMR (300 MHz, DMSO-d₆): δ 2.10 (s, 3H), 2.21 (s, 3H), 2.28 (s, 3H), 6.65 (s, 1H), 8.16 (dd, J=8.67, 1.88 Hz, 1H), 8.25 (d, J=1.88 Hz, 1H), 8.62 (d, J=8.67 Hz, 1H), 9.09 (s(br), 1H), 10.39 (s(br), 1H) ppm; HPLC-MS (Method B): m/z=422/424 (M+H), Rₜ=2.49 min.

### Example 150

### General Procedure (E)

### 4-Chloro-N-(2-chloro-4-trifluoromethanesulfonylphenyl)-2-hydroxybenzamide

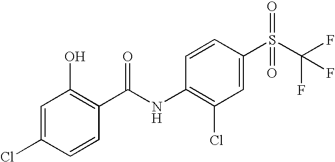

From 4-chloro-2-hydroxybenzoic acid and 2-chloro-4-(trifluoromethanesulfonyl)aniline; ¹H NMR (300 MHz, DMSO-d₆): δ 7.10 (s(br), 1H), 7.12 (dd, J=9, 1.8 Hz, 1H), 8.06 (d, J=8.67 Hz, 1H), 8.19 (dd, J=9.04, 2.26 Hz, 1H), 8.32 (d, J=2.26 Hz, 1H), 9.02 (d, J=9.04 Hz, 1H), 11.47 (s(br), 1H), 12.82 (s(br), 1H) ppm; HPLC-MS (Method B): m/z=414/416/418 (M+H), Rₜ=2.39 min.

### Example 151

### General Procedure (F)

### 2-Hydroxybiphenyl-3-carboxylic acid (2-chloro-4-trifluoromethanesulfonylphenyl)amide

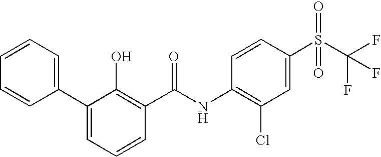

From 2-hydroxybiphenyl-3-carboxylic acid and 2-chloro-4-(trifluoromethanesulfonyl)aniline; ¹H NMR (300 MHz, DMSO-d₆): δ 7.17 (t, J=7.72 Hz, 1H), 7.40 (m, 1H), 7.48 (t, J=7.54 Hz, 2H), 7.52-7.59 (m, 3H), 8.10 (dd, J=7.7, 1.5 Hz, 1H), 8.21 (dd, J=8.85, 2.07 Hz, 1H), 8.33 (d, J=2.26 Hz, 1H), 8.68 (d, J=9.04 Hz, 1H), 11.55 (s(br), 1H), 11.63 (s(br), 1H) ppm; HPLC-MS (Method B): m/z=456/458 (M+H), Rₜ=2.62 min.

### Example 152

### General Procedure (F)

### N-(2-Chloro-4-trifluoromethanesulfonylphenyl)-2,3,4,5-tetrafluoro-6-hydroxybenzamide

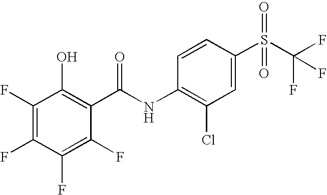

From 2,3,4,5-tetrafluoro-6-hydroxybenzoic acid and 2-chloro-4-(trifluoromethanesulfonyl)aniline; ¹H NMR (400 MHz, CDCl₃): δ 8.03 (dd, J=9.10, 2.02 Hz, 1H), 8.15 (d, J=2.02 Hz, 1H), 8.88 (d, J=9.10 Hz, 1H), 9.56 (d, J=21.73 Hz, 1H), 12.44 (s, 1H) ppm; HPLC-MS (Method B): m/z=452/454 (M+H), Rₜ=2.31 min.

### Example 153

### General Procedure (A)

### 5-tert-Butyl-4-hydroxy-2-methylbiphenyl-3-carboxylic acid (2-chloro-4-trifluoromethanesulfonylphenyl)amide

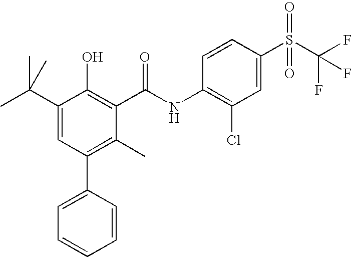

Step A: from 3-bromo-5-tert-butyl-6-hydroxy-2-methylbenzoic acid and phenylboronic acid.

Step B: from the product formed in step A, 5-tert-butyl-4-hydroxy-2-methylbiphenyl-3-carboxylic acid, and 2-chloro-4-(trifluoromethanesulfonyl)aniline; ¹H NMR (300 MHz, DMSO-d₆): δ 1.40 (s, 9H), 2.16 (s, 3H), 7.10 (s, 1H), 7.26-7.33 (m, 2H), 7.33-7.39 (m, 1H), 7.39-7.50 (m, 2H), 8.20 (d, J=8.67 Hz, 1H), 8.24 (s(br), 1H), 8.63 (d, J=8.67 Hz, 1H), 9.00 (s, 1H), 10.64 (s, 1H) ppm; HPLC-MS (Method B): m/z=426/428 (M+H), Rₜ=2.94 min.

### Example 154

### General Procedure (F)

### N-(2-Chloro-4-trifluoromethanesulfonylphenyl)-3,5-difluoro-2-hydroxybenzamide

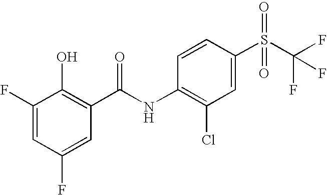

From 3,5-difluoro-2-hydroxybenzoic acid (cf. Synth. Comm. 1996, 26, 2775-2781) and 2-chloro-4-(trifluoromethanesulfonyl)aniline; ¹H NMR (300 MHz, DMSO-d₆): δ 7.53-7.74 (m, 2H), 8.20 (d(br), J=9.04 Hz, 1H), 8.32 (s(br), 1H), 8.98 (d, J=9.04 Hz, 1H), 11.64 (s(br), 1H), 12.65 (s(br), 1H) ppm; HPLC-MS (Method B): m/z=416/418 (M+H), Rₜ=2.40 min.

### Example 155

### General Procedure (A)

### 3′,5′-Difluoro-4-hydroxy-2-methylbiphenyl-3-carboxylic acid (2-chloro-4-trifluoromethanesulfonylphenyl)amide

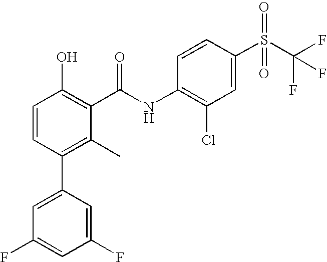

Step A: from 3-bromo-6-hydroxy-2-methylbenzoic acid (obtainable by bromination of 2-hydroxy-6-methylbenzoic acid according to the procedure described in J. Org. Chem. 1997, 62, 4504-4506) and 3,5-difluorophenylboronic acid.

Step B: from the product formed in step A, 3′,5′-difluoro-4-hydroxy-2-methylbiphenyl-3-carboxylic acid, and 2-chloro-4-(trifluoromethanesulfonyl)aniline; ¹H NMR (300 MHz, DMSO-d₆): δ 2.21 (s, 3H), 6.87 (d, J=8.29 Hz, 1H), 6.97-7.09 (m, 2H), 7.19 (d, J=8.29 Hz, 1H), 7.24 (m, 1H), 8.17 (d(br), J=8.67 Hz, 1H), 8.26 (s(br), 1H), 8.54 (d(br), J=8.67 Hz, 1H), 10.24 (s(br), 1H), 10.52 (s(br), 1H) ppm; HPLC-MS (Method B): m/z=506/508 (M+H), Rₜ=2.56 min.

### Example 156

### General Procedure (A)

### 4′-Cyano-4-hydroxy-2-methylbiphenyl-3-carboxylic acid (2-chloro-4-trifluoromethanesulfonylphenyl)amide

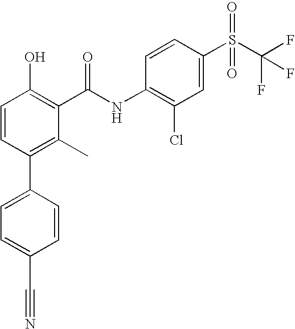

Step A: from 3-bromo-6-hydroxy-2-methylbenzoic acid (obtainable by bromination of 2-hydroxy-6-methylbenzoic acid according to the procedure described in J. Org. Chem. 1997, 62, 4504-4506) and 4-cyanophenylboronic acid.

Step B: From the product formed in step A, 4′-cyano-4-hydroxy-2-methylbiphenyl-3-carboxylic acid, and 2-chloro-4-(trifluoromethanesulfonyl)aniline; ¹H NMR (300 MHz, DMSO-d₆): δ 2.19 (s, 3H), 6.89 (d, J=8.29 Hz, 1H), 7.18 (d, J=8.29 Hz, 1H), 7.51 (d, J=7.73 Hz, 2H), 7.90 (d, J=7.73 Hz, 2H), 8.17 (d(br), J=8.67 Hz, 1H), 8.25 (s(br), 1H), 8.54 (d, J=8.67 Hz, 1H), 10.24 (s(br), 1H), 10.52 (s(br), 1H) ppm; HPLC-MS (Method B): m/z=495/497 (M+H), Rₜ=2.40 min.

### Example 157

### General Procedure (F)

### N-(2-Chloro-4-trifluoromethanesulfonylphenyl)-4-fluoro-2-hydroxybenzamide

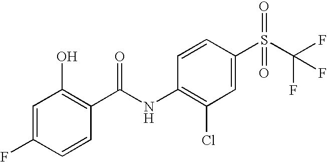

From 4-fluoro-2-hydroxybenzoic acid and 2-chloro-4-(trifluoromethanesulfonyl)aniline; ¹H NMR (300 MHz, DMSO-d₆): δ 6.84 (dd, J=10.55, 2.36 Hz, 1H), 6.91 (dt, J=8.57, 2.36 Hz, 1H), 8.12 (dd, J=9.04, 7.16 Hz, 1H), 8.18 (dd, J=8.67, 1.88 Hz, 1H), 8.31 (d, J=1.88 Hz, 1H), 9.02 (d, J=8.67 Hz, 1H), 11.40 (s, 1H), 12.85 (s(br), 1H) ppm; HPLC-MS (Method B): m/z=398/400 (M+H), Rₜ=2.34 min.

### Example 158

### General Procedure (A)

### 3′,5′-Difluoro-4-hydroxy-5-isopropyl-2-methylbiphenyl-3-carboxylic acid (2-chloro-4-trifluoromethanesulfonylphenyl)amide

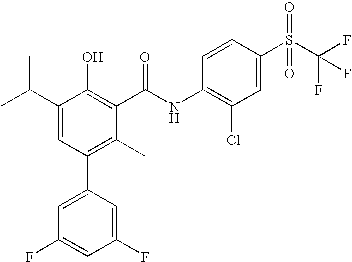

Step A: from 3-bromo-6-hydroxy-5-isopropyl-2-methylbenzoic acid (obtainable by treatment of 2-hydroxy-3-isopropyl-6-methylbenzoic acid with bromine (1.05 equiv.) in acetic acid for 18 hours at 20° C.) and 3,5-difluorophenylboronic acid.

Step B: from the product formed in step A, 3′,5′-difluoro-4-hydroxy-5-isopropyl-2-methylbiphenyl-3-carboxylic acid, and 2-chloro-4-(trifluoromethanesulfonyl)aniline; ¹H NMR (300 MHz, DMSO-d₆): δ 1.19 (d, J=6.78 Hz, 6H), 2.17 (s, 3H), 3.33 (m, J=6.78 Hz, 1H), 7.04 (m, 2H), 7.12 (s, 1H), 7.24 (tt, J=9.5, 2 Hz, 1H), 8.19 (d(br), J=8.67 Hz, 1H), 8.26 (s(br), 1H), 8.55 (d(br), J=8.67 Hz, 1H), 9.11 (s, 1H), 10.56 (s, 1H) ppm; HPLC-MS (Method B): m/z=548/550 (M+H), Rₜ=2.80 min.

### Example 159

### General Procedure (F)

### 3-tert-Butyl-6-chloro-N-(2-chloro-4-trifluoromethanesulfonylphenyl)-2-hydroxybenzamide

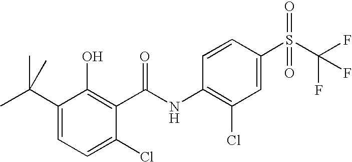

From 3-tert-butyl-6-chloro-2-hydroxybenzoic acid and 2-chloro-4-(trifluoromethanesulfonyl)aniline; ¹H NMR (400 MHz, DMSO-d₆): δ 1.37 (s, 9H), 6.96 (d, J=8.59 Hz, 1H), 7.27 (d, J=8.59 Hz, 1H), 8.20 (d(br), J=8.62 Hz, 1H), 8.24 (s(br), 1H), 8.61 (d(br), J=8.62 Hz, 1H), 9.59 (s, 1H), 10.80 (s(br), 1H) ppm; HPLC-MS (Method B): m/z=470/472 (M+H), Rₜ=2.95 min.

3-tert-Butyl-6-chloro-2-hydroxybenzoic acid may be synthesized from 2-tert-butyl-5-chlorophenol (obtainable by tert-butylation of 3-chlorophenol according to General Procedure (B)) by a modification of the procedure described in Ber. 1921, 54, 1213-1220. 2-tert-Butyl-5-chlorophenol is treated with oxalyl chloride (0.1 M 5-chlorophenol) for 72 hours at 50° C. Concentration furnishes chloro(oxo)acetic acid 2-tert-butyl-5-chlorophenyl ester as an oil, which is then dissolved in dichloromethane (concentration 0.33 M). The resulting solution is added to a suspension of aluminum chloride (3 equiv.) in dichloromethane (0.17 M final concentration) at 0° C. Stirring is continued for 20 minutes. Extractive aqueous work-up with ethyl acetate yields 7-tert-butyl-4-chlorobenzofuran-2,3-dione as a solid. The solid is then dissolved (concentration 0.12 M) in 1 M aqueous sodium hydroxide, and the mixture is cooled in an ice-water bath. 35% aqueous hydrogen peroxide (22 equiv.) is added dropwise over 5 minutes. The cooling bath is removed, and the stirring is continued at room temperature for another 2 hours. The reaction mixture is poured slowly into 1.0 M aqueous HCl, resulting in formation of a solid. Filtration and washing with water furnishes 3-tert-butyl-6-chloro-2-hydroxybenzoic acid as a solid. The crude product may be recrystallized from water-ethanol.

### Example 160

### General Procedure (F)

### 3-tert-Butyl-6-chloro-2-hydroxy-N-(2-methyl-4-trifluoromethanesulfonylphenyl)benzamide

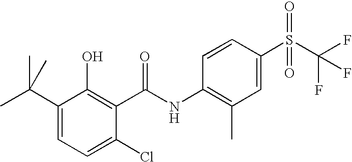

From 3-tert-butyl-6-chloro-2-hydroxybenzoic acid (the synthesis of which is described under the preparation of 3-tert-butyl-6-chloro-N-(2-chloro-4-trifluoromethanesulfonylphenyl)-2-hydroxybenzamide) and 2-methyl-4-(trifluoromethanesulfonyl)aniline; ¹H NMR (400 MHz, DMSO-d₆): δ 1.37 (s, 9H), 2.45 (s, 3H), 6.97 (d, J=8.51 Hz, 1H), 7.26 (d, J=8.51 Hz, 1H), 8.00 (s(br), 1H), 8.02 (d(br), J=7.58 Hz, 1H), 8.33 (d(br), J=7.58 Hz, 1H), 9.54 (s(br), 1H), 10.51 (s(br), 1H) ppm; HPLC-MS (Method B): m/z=450/452 (M+H), Rₜ=2.77 min.

### Example 161

### General Procedure (A)

### 5-Chloro-2-hydroxy-4-methylbiphenyl-3-carboxylic acid (2-chloro-4-trifluoromethanesulfonylphenyl)amide

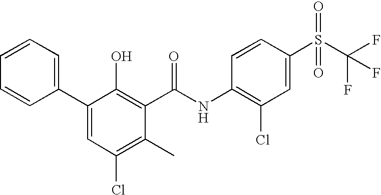

Step 1 (analogous to general procedure (A), step A): from 3-bromo-5-chloro-2-hydroxy-6-methylbenzoic acid and phenylboronic acid.

Step 2 (analogous to general procedure (A), step B): from the product formed in step 1,5-chloro-2-hydroxy-4-methylbiphenyl-3-carboxylic acid, and 2-chloro-4-(trifluoromethanesulfonyl)aniline; ¹H NMR (300 MHz, DMSO-d₆): δ 2.33 (s, 3H), 7.37 (s, 1H), 7.38 (t, J=7.4 Hz, 1H), 7.46 (t, J=7.4 Hz, 2H), 7.53 (d, J=7.4 Hz, 2H), 8.19 (d(br), J=8.63 Hz, 1H), 8.26 (s(br), 1H), 8.58 (d, J=8.63 Hz, 1H), 9.31 (s, 1H), 10.70 (s(br), 1H) ppm; HPLC-MS (Method B): m/z=504/506/508 (M+H), Rₜ=2.65 min.

3-Bromo-5-chloro-2-hydroxy-6-methylbenzoic acid may be synthesized from 3-tert-butyl-5-chloro-2-hydroxy-6-methylbenzoic acid, which upon treatment with concentrated sulfuric acid for 1 hour at room temperature yields 3-chloro-6-hydroxy-2-methylbenzoic acid. Conversion with bromine (1.15 equiv.) in acetic acid for 3 hours at 20° C. yields 3-bromo-5-chloro-2-hydroxy-6-methylbenzoic acid.

### Example 162

### General Procedure (A)

### 5-Chloro-4′-fluoro-2-hydroxy-4-methylbiphenyl-3-carboxylic acid (2-chloro-4-trifluoromethanesulfonylphenyl)amide

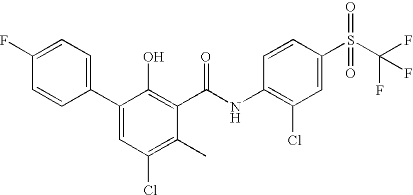

Step 1 (analogous to general procedure (A), step A): from 3-bromo-5-chloro-2-hydroxy-6-methylbenzoic acid (the synthesis of which is described under the preparation of 5-chloro-2-hydroxy-4-methylbiphenyl-3-carboxylic acid (2-chloro-4-trifluoromethanesulfonylphenyl)amide)) and 4-fluorophenylboronic acid.

Step 2 (analogous to general procedure (A), step B): from the product formed in step 1,5-chloro-4′-fluoro-2-hydroxy-4-methylbiphenyl-3-carboxylic acid, and 2-chloro-4-(trifluoromethanesulfonyl)aniline; ¹H NMR (300 MHz, DMSO-d₆): δ 2.33 (s, 3H), 7.28 (t, J=8.85 Hz, 2H), 7.38 (s, 1H), 7.56 (dd, J=8.48, 5.46 Hz, 2H), 8.19 (d(br), J=8.86 Hz, 1H), 8.26 (s(br), 1H), 8.57 (d, J=8.86 Hz, 1H), 9.35 (s, 1H), 10.70 (s(br), 1H) ppm; HPLC-MS (Method B): m/z=522/524/526 (M+H), Rₜ=2.66 min.

### Example 163

### General Procedure (C)

### N-(2-Chloro-4-trifluoromethanesulfonyl-phenyl)-6-hydroxy-2,4-dimethyl-3-methylsulfanyl-benzamide

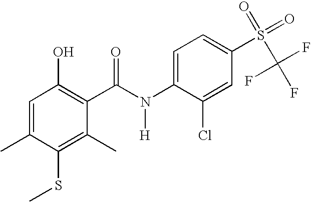

From 3,5-dimethyl-4-methylsulfanyl-phenol and 2-chloro-4-trifluoromethanesulfonyl-aniline; mp 201-202° C.; ¹H NMR (CDCl₃): δ 9.00 (d, J=9.10 Hz, 1H), 8.97 (s, 1H), 8.54 (br s, 1H), 8.09 (d, J=2.02 Hz, 1H), 8.00 (dd, J=8.59 and 2.02 Hz, 1H), 6.83 (s, 1H), 2.89 (s, 3H), 2.58 (s, 3H), 2.22 (s, 3H); HPLC-MS (Method B): m/z 454/456 (M+H), 476/478 (M+Na), Rₜ 2.48 min.

### Example 164

### General Procedure (H), Step D

### N-(2-Chloro-4-trifluoromethanesulfonyl-phenyl)-6-hydroxy-3-methanesulfinyl-2,4-dimethyl-benzamide

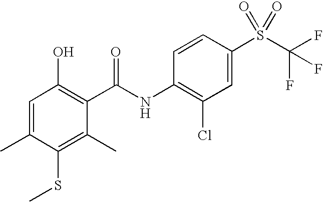

From N-(2-chloro-4-trifluoromethanesulfonyl-phenyl)-6-hydroxy-2,4-dimethyl-3-methylsulfanyl-benzamide; white crystals, mp 205-206° C.; HPLC-MS (Method B): m/z 470/472 (M+H), 492/494 (M+Na), Rₜ 1.73 min.

### Pharmacological Methods

Assay (I): Glucose Utilisation in a Human Epithelia Cell Line (FSK-4 Cells)

### Assay Description:

The assay measures indirectly the activity of the respiratory chain in FSK-4 cells by using D-(6-3H(N))-glucose. The ³H-proton will first be released in the TCA cyclus and transported to the respiratory chain where it will be incorporated into water. The water is thereafter separated from the D-(6-³H(N))-glucose by evaporation. Finally, the radioactivity in the water is determined using a Topcounter.

### Method:

FSK-4 cells obtained from ATCC (Maryland, USA), are cultured in growth medium (McCoy's medium with the following addition 100 units/ml penicillin and streptomycin and 10% FCS (fetal calf serum)) at 37° C. and 5% CO₂. All media are obtained by Gibco (Life Technologies, Maryland, USA) where not otherwise mentioned.

At day zero the cells are harvested using trypsin-EDTA and washed in assay medium (MEM medium with the following addition 1× non-essential amino acids (M7145, 2 mM glutamin, 100 units/ml penicillin and streptomycin, 0.0075% sodium bicarbonate, 1 mM sodium pyrovate and 2% horse serum) using centrifugation. The cells are plated into single StripPlates wells (Corning B.V. Life Sciences, The Netherlands) that are placed into 24-well plates (Corning B.V. Life Sciences, The Netherlands) with a concentration of 1, 5×10⁴ cells/100 μl assay medium/well. The cells are then incubated at 37° C. and 5% CO₂ overnight.

The next day the compounds to be tested are diluted to different concentrations in DMSO (Sigma, Mo., USA) to 100 times final concentration. They are then diluted to a final concentration in assay medium containing 10 μCi/ml D-(6-³H(N))-glucose (PerkinElmer Life Sciences Inc., Boston, USA). The medium is removed from the cells and 200 μl of the compound dilutions are added in duplicates. The cells are then incubated for another 24 hours at 37° C. and 5% CO₂. Finally the cells are lysed by adding 50 μl 10% TCA (trichloroacetate). 300 μl of sterile water is then added to the 24-wells that surrounds the StripPlate wells. The plate is sealed with Top-seal-tape (Packard, PerkinElmer Life Sciences Inc., Boston, USA) and the plate is incubated in a heating cupboard at 50° C. to equilibrium the radioactive water formed in the respiratory chain into the water in the 24-well plate by evaporate. The plates incubate for 8 hours where the heating cupboard is turned off. The top seal is removed when the samples have reached room temperature. One ml scintillation liquid (Packard Microscient, PerkinElmer Life Sciences Inc., Boston, USA) is added to all the samples and the radioactivity is determined using a Topcounter (Packard, PerkinElmer Life Sciences Inc., Boston, USA). Non-specific activity is determined by evaporating 200 μl of the dilution medium containing the D-(6-³H(N))-glucose into 300 μl sterile water, and total radioactivity is determined by counting 5 μl assay medium with 10 μCi/ml D-(6-3H(N))-glucose.

### Calculations

The half maximal concentration (EC₅₀) and maximal efficacy (Eₘₐₓ) are calculated using the Hill equation in GraphPad Prism 3.0 (GraphPad software, Inc.). In studies where the linear slope is determined the following concentration of the compound is used; 5×, 3×, 2×, 1, 5×, 1, 25×, 1×, 0.85×, 0.7×, 0.5×, 0.3×, 0.2× and 0×EC₅₀. From the percentage increase in glucose utilisation the linear slope is calculated using the Michaelis-Menten equation.

### Assay (II): The Effect of Chemical Uncouplers on Mitochondrial Respiration Using Isolated Mitochondria.

This assay is used to investigate if the increase in glucose utilisation caused by the test compounds observed in the glucose utilisation assay is due to an increase in the respiration of the mitochondria. This is done by measuring oxygen consumption in isolated rat liver mitochondria.

A Clark oxygen electrode is used to determine the oxygen consumption. The isolated mitochondria are added to assay medium (D-Mannitol 220 mM, MagnesiumCloride 5 mM, HEPES 2 mM and PotassiumPhosphate 5 mM, pH=7, 4) containing rotenone (an inhibitor of clomplex 1) and oligomyocin (an inhibitor of the ATP-synthase) and the rate of oxygen consumptions is measured, when stabilized nutrient (e.g. succinate) is added and an increase in the rate of oxygen consumption is measured. When the rate of oxygen consumption again has stabilized the test compound is added and the oxygen consumption is measured. If the test compound stimulates the rate of oxygen consumption, it is regarded as a chemical uncoupler.

Assay (III): Identification of Chemical Uncouplers that Increase Energy Expenditure in Vivo

The effect of the chemical uncouplers on energy expenditure (oxygen consumption) in vivo is determined by indirect calorimetry. Briefly, animals are placed in airtight chambers. Air is continuously led to and from the chambers. The gas concentrations of oxygen (O₂) and carbondioxide (CO₂) in the air led to and from the chambers (inlet and outlet air) are recorded and the consumption of O₂ and the production of CO₂ are calculated. Based on the amount of O₂ consumed and CO₂ produced, energy expenditure is calculated. Compounds which at a given dose increase whole body energy expenditure without obvious deleterious effects are deemed to be chemical uncouplers that increase energy expenditure.

1. A compound according to formula I 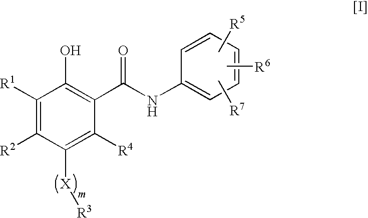
wherein
m is 0, such that R³ is linked directly to the benzene ring by a bond, or
m is 1 or 2; 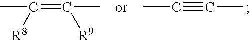
X, when present, represents a group
R¹ represents hydrogen or halogen; or R¹ represents C₁₋₈alkyl, C₁₋₆alkenyl, C₁₋₆alkynyl, C₃₋₈cycloalkyl, C₄₋₈cycloalkenyl or phenyl, all of which may optionally be further substituted by halogen, C₁₋₈alkyl, C₃₋₈cycloalkyl, C₄₋₈cycloalkenyl or phenyl; or R¹ represents bicyclo-C₄₋₁₀alkyl or tricyclo-C₄₋₁₀-alkyl; wherein said C₃₋₈cycloalkyl, bicyclo-C₄₋₁₀alkyl, tricyclo-C₄₋₁₀alkyl or phenyl are optionally substituted with one or more substituents selected among hydroxy, cyano, nitro, C₁₋₆alkyl, C₁₋₆alkenyl, C₁₋₆alkynyl, C₃₋₈-cycloalkyl, C₄₋₈cycloalkenyl, C₁₋₆alkoxy, C₁₋₆haloalkoxy and C₁₋₆haloalkyl;
R² represents hydrogen, halogen, C₁₋₆alkyl, C₁₋₆alkenyl, C₁₋₆alkynyl, C₃₋₈-cycloalkyl, C₄₋₈cycloalkenyl or C₁₋₆alkoxy;
R⁴ represents, hydrogen, halogen, hydroxy, C₁₋₆alkyl, C₁₋₆alkenyl, C₁₋₆alkynyl, C₃₋₈-cycloalkyl, C₄₋₈cycloalkenyl or C₁₋₁₆alkoxy;
R⁸ and R⁹ independently represent, hydrogen, C₁₋₆alkyl, C₁₋₆alkenyl, C₁₋₆alkynyl, C₃₋₈-cycloalkyl, C₄₋₈cycloalkenyl or C₁₋₆alkoxy;
or
R¹ and R² together with the benzene ring, or, with the proviso that m is 0, R² and R³ together with the benzene ring or R³ and R⁴ together with the benzene ring form a 9-11-membered bicyclic ring system which may be fully conjugated or partly saturated, and which may optionally be substituted with one or more substituents selected among halogen, hydroxy, nitro, cyano, C₁₋₆alkyl, C₁₋₆-alkenyl, C₁₋₆alkynyl, C₃₋₈cycloalkyl, C₄₋₈ cycloalkenyl, C₁₋₆alkoxy, C₁₋₆haloalkoxy and C₁₋₆-haloalkyl;
at least one substituent among R⁵, R⁶ and R⁷ represents —SR¹², —S(O)R¹² or —S(O)₂R¹², and the remaining substituents among R⁵, R⁶ and R⁷ independently represent hydrogen, nitro, cyano, halogen, C₁₋₆alkyl, C₁₋₆alkenyl, C₁₋₆alkynyl, C₃₋₈cycloalkyl, C₄₋₈cycloalkenyl, C₁₋₆haloalkyl, —OR¹⁰, —NR¹⁰R¹¹, —C(O)OR¹⁰, —COR¹⁰, —C(O)NR¹⁰R¹¹, —SH, —S(O)₂OR¹⁰, —S(O)₂NR¹⁰R¹¹, —SR¹¹, —S(O)R¹¹, —S(O)₂R¹¹, aryl or heteroaryl; wherein said aryl or heteroaryl may optionally be substituted with one or more substituents selected among C₁₋₆alkyl, halogen, hydroxy and phenyl;
R¹⁰ and R¹¹ independently represent hydrogen, C₁₋₆alkyl, C₁₋₆alkenyl, C₁₋₆alkynyl, C₃₋₈-cycloalkyl, C₄₋₈cycloalkenyl, C₁₋₁₆haloalkyl or C₁₋₁₆haloalkoxy;
R¹² represents C₁₋₆haloalkyl;
R³ represents hydrogen, amino, nitro, cyano, halogen, C₁₋₆alkyl, C₁₋₆alkenyl, C₁₋₆alkynyl, C₁₋₆-haloalkyl, arylC₁₋₆alkyl, arylC₁₋₆alkenyl, arylC₁₋₆alkynyl, heteroarylC₁₋₆alkyl, heteroarylC₁₋₆-alkenyl, heteroarylC₁₋₆alkynyl, C₃₋₈cycloalkyl, —OR¹⁷, —NR¹⁷R¹⁸, C₁₋₆haloalkyl, —C(O)OR¹⁷, —COR⁷, —C(O)NR¹⁷R¹⁸, —SH, —S(O)₂OR¹⁷, —S(O)₂NR¹⁷R¹⁸, —SR¹⁷, —S(O)R¹⁷, —S(O)₂R¹⁷, —NH—COR¹⁷ or —NH—S(O)₂R¹⁷; or R³ represents aryl or heteroaryl which may optionally be substituted with one, two, three or four substituents independently selected among halogen, cyano, nitro, C₁₋₆alkyl, C₁₋₆alkenyl, C₁₋₆alkynyl, C₃₋₈cycloalkyl, C₄₋₈cycloalkenyl, C₁₋₆alkoxy, C₁₋₆haloalkoxy, C₁₋₆haloalkyl, C₁₋₆hydroxyalkyl, C₁₋₆alkylaryl, hydroxy, —(CH₂)ᵣOR¹³, —SH, —S(O)ₚR¹³, —S(O)ₚN(R¹³)(R¹⁴), —C(O)OR¹³, —OC(O)R¹³, —C(O)R¹³, —C(O)N(R¹³)(R¹⁴), —(CH₂)ᵣN(R¹³)C(O)R¹⁴, —B(OR¹³)(OR¹⁴), —(CH₂)ᵣN(R¹³)(R¹⁴) and phenyl; wherein said phenyl is optionally substituted with one or more substituents selected among cyano, nitro, C₁₋₆alkyl, halogen, C₁₋₆haloalkyl, C₁₋₆hydroxyalkyl, C₁₋₆haloalkoxy, —OR¹⁵—, —S(O)ₛR⁵, —C(O)OR¹⁵, —OC(O)R¹⁵, —C(O)R¹⁵, —C(O)N(R¹⁵)(R¹⁶), —N(R¹⁵)(R¹⁶), —(CH₂)ₛN(R¹⁵)C(O)R¹⁶, —B(OR¹⁵)(OR¹⁶), —(CH₂)ₜOR¹⁵ and —(CH₂)ₜN(R¹⁵)(R¹⁶);
R¹³, R¹⁴, R¹⁵, R¹⁶ and R¹⁸ independently represent hydrogen, C₁₋₆alkyl, C₁₋₆alkenyl, C₁₋₆alkynyl, C₃₋₈cycloalkyl, C₄₋₈cycloalkenyl, C₁₋₆haloalkyl, C₁₋₆hydroxyalkyl, C₁₋₆-aminoalkyl or phenyl, said phenyl optionally being substituted with one or more substituents selected among halogen, cyano, C₁₋₆alkyl, C₁₋₆alkenyl, C₁₋₆alkynyl, C₃₋₈cycloalkyl, C₄₋₈cycloalkenyl, C₁₋₆haloalkyl, C₁₋₆ haloalkoxy and C₁₋₆hydroxyalkyl;
R¹⁷ represents hydrogen, C₁₋₆alkyl, C₁₋₆alkenyl, C₁₋₆alkynyl, C₃₋₈cycloalkyl, C₄₋₈cycloalkenyl, C₁₋₆haloalkyl, C₁₋₆hydroxyalkyl, C₁₋₆-aminoalkyl, arylC₁₋₆alkyl or phenyl, said phenyl optionally being substituted with one or more substituents selected among halogen, cyano, C₁₋₆alkyl, C₁₋₆alkenyl, C₁₋₆alkynyl, C₃₋₈cycloalkyl, C₄₋₈cycloalkenyl, C₁₋₆-haloalkyl, C₁₋₆haloalkoxy and
C₁₋₆hydroxyalkyl;
or one or more of the substituent pairs R¹⁰ and R¹¹, R¹³ and R¹⁴, R¹⁵ and R¹⁶, and R¹⁷ and R¹⁸, when attached to the same nitrogen atom, together with said nitrogen atom form a saturated or unsaturated carbocyclic or heterocyclic 3-8-membered ring, optionally substituted with one or more C₁₋₁₆alkyl substituents;
p and s, independently of each other, are 0, 1 or 2;
r and t, independently of each other, are 0, 1, 2 or 3;
and pharmaceutically acceptable salts, solvates and prodrugs thereof.

2. A compound according to claim 1, wherein R¹ represents tert-butyl.

3. A compound according to claim 1, wherein R¹ represents hydrogen, halogen or halogen-substituted phenyl.

4. A compound according to claim 1, wherein R¹² represents C₁₋₆-fluoroalkyl.

5. A compound according to claim 4, wherein R¹² represents trifluoromethyl.

6. A compound according to claim 1, wherein R¹ and R² together with the benzene ring, or, with the proviso that m is 0, R² and R³ together with the benzene ring or
R³ and R⁴ together with the benzene ring form a 9-11-membered bicyclic ring system which may be fully conjugated or partly saturated, and which may optionally be substituted with one or more substituents selected among halogen, hydroxy, nitro, cyano, C₁₋₆alkyl, C₁₋₆-alkenyl, C₁₋₆alkynyl, C₃₋₈cycloalkyl, C₄₋₈cycloalkenyl, C₁₋₆alkoxy, C₁₋₁₆haloalkoxy and C₁₋₁₆haloalkyl.

7. A compound according to claim 6, wherein R¹ and R² together with the benzene ring form a naphthalene ring system.

8. A compound according to claim 6, wherein m is 0, and R³ and R⁴ together with the benzene ring form a tetrahydronaphthalene ring system.

9. A compound according to claim 6, wherein m is 0, and R³ and R⁴ together with the benzene ring form an indane ring system.

10. A compound according to claim 1, wherein R² represents halogen.

11. A compound according to claim 1, wherein m is 0, and R³ represents —SR¹⁷, —S(O)R¹⁷ or —S(O)₂R¹⁷.

12. A compound according to claim 11, wherein R¹⁷ represents arylC₁₋₆alkyl.

13. A compound according to claim 1, wherein m is 0, and R³ represents —SCH₃, —S(O)CH₃ or —S(O)₂CH₃.

14. A compound according to claim 1, wherein m is 0, and R³ represents —OR¹⁷.

15. A compound according to claim 1, wherein m is 0, and R³ represents —NH₂, —NH—COR¹⁷ or —NH—S(O)₂R¹⁷.

16. A compound according to claim 1, wherein m is 0, and R³ represents chlorine or fluorine.

17. A compound according to claim 1, wherein m is 0, and R³ represents —S(O)₂NR¹⁷R¹⁸.

18. A compound according to claim 17, wherein NR¹⁷R¹⁸ represents morpholin-4-yl.

19. A compound according to claim 1, wherein m is 0, and R³ represents optionally substituted aryl or heteroaryl.

20. A compound according to claim 19, wherein R³ represents optionally substituted aryl.

21. A compound according to claim 19, wherein R³ represents optionally substituted phenyl.

22. A compound according to claim 21, wherein R³ is phenyl substituted with one or more substituents selected among halogen, cyano, C₁₋₆alkyl, C₁₋₆haloalkyl, C₁₋₆haloalkoxy, C₁₋₆alkoxy, —S(O)ₚR¹⁷ and —C(O)—R¹⁷.

23. A compound according to claim 1, wherein R⁴ represents halogen or hydroxy.

24. A compound according to claim 1, wherein R¹ represents tert-butyl, and R⁴ represents C₁₋₆alkyl.

25. A compound according to claim 1, wherein one substituent among R⁵, R⁶ and R⁷ represents —S(O)₂CF₃, and one of the remaining substituents among R⁵, R⁶ and R⁷ represents chloro.

26. A compound according to claim 1, wherein one substituent among R⁵, R⁶ and R⁷ represents 4-trifluoromethylsulfonyl, and one of the remaining substituents among R⁵, R⁶ and R⁷ represents 2-chloro.

27. A compound according to claim 26, wherein the single remaining substituent among R⁵, R⁶ and R⁷ represents hydrogen.

28. A compound according to claim 1, selected from the group consisting of:
5-tert-Butyl-3′,5′-difluoro-4-hydroxy-2-methyl-biphenyl-3-carboxylic acid (2-chloro-4-trifluoromethanesulfonyl-phenyl)-amide;
5-tert-Butyl-4-hydroxy-2-methyl-4′-trifluoromethoxy-biphenyl-3-carboxylic acid (2-chloro-4-trifluoromethanesulfonyl-phenyl)-amide;
5-tert-Butyl-4-hydroxy-2-methyl-3′-trifluoromethyl-biphenyl-3-carboxylic acid (2-chloro-4-trifluoromethanesulfonyl-phenyl)-amide;
3-Bromo-5-tert-butyl-N-(2-chloro-4-trifluoromethanesulfonyl-phenyl)-6-hydroxy-2-methyl-benzamide;
5-tert-Butyl-4′-fluoro-4-hydroxy-2-methyl-biphenyl-3-carboxylic acid (2-chloro-4-trifluoromethanesulfonyl-phenyl)-amide;
5-tert-Butyl-4-hydroxy-2-methyl-4′-methylsulfanyl-biphenyl-3-carboxylic acid (2-chloro-4-trifluoromethanesulfonyl-phenyl)-amide;
5-tert-Butyl-3′,4′-difluoro-4-hydroxy-2-methyl-biphenyl-3-carboxylic acid (2-chloro-4-trifluoromethanesulfonyl-phenyl)-amide;
5-tert-Butyl-4′-cyano-4-hydroxy-2-methyl-biphenyl-3-carboxylic acid (2-chloro-4-trifluoromethanesulfonyl-phenyl)-amide;
3-Bromo-5-tert-butyl-N-(3-chloro-4-trifluoromethylsulfanyl-phenyl)-6-hydroxy-2-methyl-benzamide;
5-tert-Butyl-3′,4′-difluoro-4-hydroxy-2-methyl-biphenyl-3-carboxylic acid (3-chloro-4-trifluoromethylsulfanyl-phenyl)-amide;
5-tert-Butyl-3′-chloro-4′-fluoro-4-hydroxy-2-methyl-biphenyl-3-carboxylic acid (2-chloro-4-trifluoromethanesulfonyl-phenyl)-amide;
5-tert-Butyl-4′-fluoro-4-hydroxy-2,3′-dimethyl-biphenyl-3-carboxylic acid (2-chloro-4-trifluoromethanesulfonyl-phenyl)-amide;
3-Chloro-5-tert-butyl-N-(2-chloro-4-trifluoromethanesulfonyl-phenyl)-6-hydroxy-2-methyl-benzamide;
5-tert-Butyl-4-hydroxy-4′-methanesulfonyl-2-methyl-biphenyl-3-carboxylic acid (2-chloro-4-trifluoromethanesulfonyl-phenyl)-amide;
5-tert-Butyl-3′,4′-difluoro-4-hydroxy-2-methyl-biphenyl-3-carboxylic acid (3-chloro-4-trifluoromethanesulfinyl-phenyl)-amide;
1-Hydroxy-naphthalene-2-carboxylic acid (3-trifluoromethanesulfonyl-phenyl)-amide;
1-Hydroxy-naphthalene-2-carboxylic acid (3-trifluoromethylsulfanyl-phenyl)-amide;
2-Hydroxy-naphthalene-1-carboxylic acid (3-trifluoromethanesulfonyl-phenyl)-amide;
3-Hydroxy-naphthalene-2-carboxylic acid (3-trifluoromethanesulfonyl-phenyl)-amide;
1-Hydroxy-5,6,7,8-tetrahydro-naphthalene-2-carboxylic acid (2-chloro-4-trifluoromethanesulfonyl-phenyl)-amide;
1-Hydroxy-5,6,7,8-tetrahydro-naphthalene-2-carboxylic acid (4-trifluoromethylsulfanylphenyl)-amide;
4-Benzenesulfonyl-1-hydroxy-naphthalene-2-carboxylic acid (4-trifluoromethylsulfanylphenyl)-amide;
4-Chloro-1-hydroxy-naphthalene-2-carboxylic acid (4-trifluoromethylsulfanyl-phenyl)amide;
3-Adamantan-1-yl-N-(2-chloro-4-trifluoromethanesulfonyl-phenyl)-2-hydroxy-5-methylbenzamide;
4-Chloro-1-hydroxy-naphthalene-2-carboxylic acid (2-chloro-4-trifluoromethanesulfonyl-phenyl)-amide;
3-tert-Butyl-N-(2-chloro-4-trifluoromethanesulfonyl-phenyl)-2-hydroxy-5-methylbenzamide;
4-Chloro-1-hydroxy-naphthalene-2-carboxylic acid (3-chloro-4-trifluoromethylsulfanyl-phenyl)-amide;
3-Adamantan-1-yl-N-(3-chloro-4-trifluoromethylsulfanyl-phenyl)-2-hydroxy-5-methylbenzamide;
3-Adamantan-1-yl-N-(3-chloro-4-trifluoromethylsulfanyl-phenyl)-2-hydroxy-5-methylbenzamide;
6-Adamantan-1-yl-5-hydroxy-indan-4-carboxylic acid (2-chloro-4-trifluoromethanesulfonyl-phenyl)-amide;
4-(4-Fluoro-phenylsulfanyl)-1-hydroxy-naphthalene-2-carboxylic acid (2-chloro-4-trifluoromethanesulfonyl-phenyl)-amide;
5-Adamantan-1-yl-2′,4′-difluoro-4-hydroxy-biphenyl-3-carboxylic acid (2-chloro-4-trifluoromethanesulfonyl-phenyl)-amide;
4-(4-Fluoro-benzenesulfinyl)-1-hydroxy-naphthalene-2-carboxylic acid (2-chloro-4-trifluoromethanesulfonyl-phenyl)-amide;
5-tert-Butyl-2′,4′-difluoro-4-hydroxy-biphenyl-3-carboxylic acid (2-chloro-4-trifluoromethanesulfonyl-phenyl)-amide;
2′,4′-Difluoro-4-hydroxy-5-(1-methyl-cyclohexyl)-biphenyl-3-carboxylic acid (2-chloro-4-trifluoromethanesulfonyl-phenyl)-amide;
4-(4-Ethanesulfonyl-phenyl)-1-hydroxy-naphthalene-2-carboxylic acid (2-chloro-4-trifluoromethanesulfonyl-phenyl)-amide;
1-Hydroxy-4-(4-methoxy-phenyl)-naphthalene-2-carboxylic acid (2-chloro-4-trifluoromethanesulfonyl-phenyl)-amide;
4-(4-Fluoro-phenyl)-1-hydroxy-naphthalene-2-carboxylic acid (2-chloro-4-trifluoromethanesulfonyl-phenyl)-amide;
1-Hydroxy-4-p-tolyl-naphthalene-2-carboxylic acid (2-chloro-4-trifluoromethanesulfonyl-phenyl)-amide;
4-(4-Cyano-phenyl)-1-hydroxy-naphthalene-2-carboxylic acid (2-chloro-4-trifluoromethanesulfonyl-phenyl)-amide;
4-(3,5-Difluoro-phenyl)-1-hydroxy-naphthalene-2-carboxylic acid (2-chloro-4-trifluoromethanesulfonyl-phenyl)-amide;
1-Hydroxy-4-(3-trifluoromethyl-phenyl)-naphthalene-2-carboxylic acid (2-chloro-4-trifluoromethanesulfonyl-phenyl)-amide;
4-(4-Acetyl-phenyl)-1-hydroxy-naphthalene-2-carboxylic acid (2-chloro-4-trifluoromethanesulfonyl-phenyl)-amide;
2-Hydroxy-5-methyl-3-(1,1,2,2-tetramethyl-propyl)-N-(4-trifluoromethanesulfonylphenyl)-benzamide;
4′-Bromo-5-tert-butyl-4-hydroxy-biphenyl-3-carboxylic acid (2-chloro-4-trifluoromethanesulfonyl-phenyl)-amide;
5-tert-Butyl-2′,4′-difluoro-4-hydroxy-biphenyl-3-carboxylic acid (4-trifluoromethanesulfonyl-phenyl)-amide;
2′,4′-Difluoro-4-hydroxy-5-(1-methyl-cyclopentyl)-biphenyl-3-carboxylic acid (4-trifluoromethanesulfonyl-phenyl)-amide;
5-(1,1-Dimethyl-propyl)-2′,4′-difluoro-4-hydroxy-biphenyl-3-carboxylic acid (2-chloro-4-trifluoromethanesulfonyl-phenyl)-amide;
5-(1-Cyclopropyl-1-methyl-ethyl)-2′,4′-difluoro-4-hydroxy-biphenyl-3-carboxylic acid (2-chloro-4-trifluoromethanesulfonyl-phenyl)-amide;
5-Chloro-N-(2-chloro-4-trifluoromethanesulfonyl-phenyl)-3-(1,1-dimethyl-propyl)-2-hydroxy-benzamide;
N-(2-Chloro-4-trifluoromethanesulfonyl-phenyl)-2-hydroxy-5-methanesulfonyl-3-(1,1,2,2-tetramethyl-propyl)-benzamide;
3-tert-Butyl-N-(2-chloro-4-trifluoromethanesulfonyl-phenyl)-2-hydroxy-5-methanesulfonyl-6-methyl-benzamide;
3-tert-Butyl-N-(2-chloro-4-trifluoromethanesulfonyl-phenyl)-6-ethyl-2-hydroxybenzamide;
3,5-Di-tert-butyl-N-(2-chloro-4-trifluoromethanesulfonyl-phenyl)-2-ethyl-6-hydroxybenzamide;
6-tert-Butyl-5-hydroxy-indan-4-carboxylic acid (2-chloro-4-trifluoromethanesulfonylphenyl)-amide;
3-tert-Butyl-N-(2-chloro-4-trifluoromethanesulfonyl-phenyl)-2-hydroxy-6-methyl-5-methylsulfanyl-benzamide;
3-tert-Butyl-2-hydroxy-5,6,7,8-tetrahydro-naphthalene-1-carboxylic acid (2-chloro-4-trifluoromethanesulfonyl-phenyl)-amide;
3-tert-Butyl-N-(2-chloro-4-trifluoromethanesulfonyl-phenyl)-2-hydroxy-5-methanesulfinyl-6-methyl-benzamide;
3-tert-Butyl-N-(2-chloro-4-trifluoromethanesulfonyl-phenyl)-2-hydroxy-5-methoxy-benzamide
3-tert-Butyl-N-(2-chloro-4-trifluoromethanesulfonyl-phenyl)-2-hydroxy-6-isopropyl-benzamide
5-tert-Butyl-3′,5′-difluoro-4-hydroxy-2-methyl-biphenyl-3-carboxylic acid (4-trifluoromethanesulfonyl-phenyl)-amide;
5-tert-Butyl-4′-fluoro-4-hydroxy-2-methyl-biphenyl-3-carboxylic acid (4-trifluoromethanesulfonyl-phenyl)-amide;
5-tert-Butyl-3′,5′-difluoro-4-hydroxy-2-methyl-biphenyl-3-carboxylic acid (3-chloro-4-trifluoromethanesulfonyl-phenyl)-amide;
5-tert-Butyl-4′-fluoro-4-hydroxy-2-methyl-biphenyl-3-carboxylic acid (3-chloro-4-trifluoromethanesulfonyl-phenyl)-amide;
3-tert-Butyl-N-(2-chloro-4-trifluoromethanesulfonyl-phenyl)-5-fluoro-2-hydroxy-6-methyl-benzamide;
3-tert-Butyl-5-chloro-N-(3-chloro-4-trifluoromethanesulfonyl-phenyl)-2-hydroxy-6-methyl-benzamide;
3-tert-Butyl-N-(2-chloro-4-trifluoromethanesulfonyl-phenyl)-2-hydroxy-5-iodo-6-methyl-benzamide;
3-tert-Butyl-5-chloro-2-hydroxy-6-methyl-N-(4-trifluoromethanesulfonyl-phenyl)benzamide;
5-tert-Butyl-3′,4′-difluoro-4-hydroxy-2-methyl-biphenyl-3-carboxylic acid (4-trifluoromethanesulfonyl-phenyl)-amide;
5-tert-Butyl-3′,4′-difluoro-4-hydroxy-2-methyl-biphenyl-3-carboxylic acid □(3-chloro-4-trifluoromethanesulfonyl-phenyl)-amide;
5-tert-Butyl-4-hydroxy-4′-methoxy-2-methyl-biphenyl-3-carboxylic acid (2-chloro-4-trifluoromethanesulfonyl-phenyl)-amide;
3-tert-Butyl-N-(2-chloro-4-trifluoromethanesulfonyl-phenyl)-5-(5-cyano-thiophen-2-yl)-2-hydroxy-6-methyl-benzamide;
5-tert-Butyl-4′-dimethylsulfamoyl-4-hydroxy-2-methyl-biphenyl-3-carboxylic acid (2-chloro-4-trifluoromethanesulfonyl-phenyl)-amide;
5-tert-Butyl-4-hydroxy-2-methyl-4′-(morpholine-4-sulfonyl)-biphenyl-3-carboxylic acid (2-chloro-4-trifluoromethanesulfonyl-phenyl)-amide;
3-1,3-Benzodioxol-5-yl-5-tert-butyl-N-(2-chloro-4-trifluoromethanesulfonyl-phenyl)-6-hydroxy-2-methyl-benzamide;
5-tert-Butyl-4-hydroxy-3′-methoxy-2-methyl-biphenyl-3-carboxylic acid (2-chloro-4-trifluoromethanesulfonyl-phenyl)-amide;
4-(3,4-Difluorophenyl)-1-hydroxynaphthalene-2-carboxylic acid (2-chloro-4-trifluoromethanesulfonylphenyl)amide;
1-Hydroxy-4-(4-trifluoromethylphenyl)naphthalene-2-carboxylic acid (2-chloro-4-trifluoromethanesulfonylphenyl)amide;
1-Hydroxy-4-(3-methoxyphenyl)naphthalene-2-carboxylic acid (2-chloro-4-trifluoromethanesulfonylphenyl)amide;
4-Bromo-1-hydroxynaphthalene-2-carboxylic acid (2-chloro-4-trifluoromethanesulfonylphenyl)amide;
5-Bromo-3-tert-butyl-N-(2-chloro-4-trifluoromethanesulfonylphenyl)-2-hydroxybenzamide;
5-tert-Butyl-3′,4′-difluoro-4-hydroxybiphenyl-3-carboxylic acid (2-chloro-4-trifluoromethanesulfonylphenyl)amide;
5-tert-Butyl-4′-fluoro-4-hydroxybiphenyl-3-carboxylic acid (2-chloro-4-trifluoromethanesulfonylphenyl)amide;
5-tert-Butyl-3′,5′-difluoro-4-hydroxybiphenyl-3-carboxylic acid (2-chloro-4-trifluoromethanesulfonylphenyl)amide;
3,5-Di-tert-butyl-N-(2-chloro-4-trifluoromethanesulfonylphenyl)-2-hydroxybenzamide;
3-tert-Butyl-N-(2-chloro-4-trifluoromethanesulfonylphenyl)-2-hydroxy-6-methylbenzamide;
1-Hydroxy-3-methylnaphthalene-2-carboxylic acid (2-chloro-4-trifluoromethanesulfonylphenyl)amide;
4-Bromo-1-hydroxy-3-methylnaphthalene-2-carboxylic acid (2-chloro-4-trifluoromethanesulfonylphenyl)amide;
3-Bromo-5-tert-butyl-6-hydroxy-2-methyl-N-(2-nitro-4-trifluoromethanesulfonylphenyl)-benzamide;
5-tert-Butyl-3′-fluoro-4-hydroxy-2-methyl-biphenyl-3-carboxylic acid (2-chloro-4-trifluoromethanesulfonyl-phenyl)-amide;
5′-tert-Butyl-3′-(2-chloro-4-trifluoromethanesulfonyl-phenylcarbamoyl)-4′-hydroxy-2′-methyl-biphenyl-3-carboxylic acid methyl ester;
3-tert-Butyl-N-(3-chloro-4-trifluoromethanesulfonyl-phenyl)-5-fluoro-2-hydroxy-6-methyl-benzamide;
3-tert-Butyl-5-chloro-N-(2-dimethylamino-4-trifluoromethanesulfonyl-phenyl)-2-hydroxy-6-methyl-benzamide;
3-tert-Butyl-5-chloro-N-(2-dipropylamino-4-trifluoromethanesulfonyl-phenyl)-2-hydroxy-6-methyl-benzamide;
3-tert-Butyl-(2-chloro-4-trifluoromethanesulfonyl-phenyl)-2-hydroxy-6-methyl-5-(morpholine-4-sulfonyl)-benzamide;
3-tert-Butyl-N-(2-chloro-4-trifluoromethanesulfonylphenyl)-5-dimethylsulfamoyl-2-hydroxy-6-methyl-benzamide;
3-tert-Butyl-N-(2-chloro-4-trifluoromethanesulfonylphenyl)-5-dimethylsulfamoyl-2-hydroxy-6-methyl-benzamide;
3-tert-Butyl-N-(2-chloro-4-trifluoromethanesulfonyl-phenyl)-6-ethyl-2-hydroxy-5-methylsulfanyl-benzamide;
3-tert-Butyl-N-(2-chloro-4-trifluoromethanesulfonyl-phenyl)-6-ethyl-2-hydroxy-5-methanesulfinyl-benzamide;
3-tert-Butyl-N-(2-chloro-4-trifluoromethanesulfonyl-phenyl)-6-ethyl-2-hydroxy-5-methanesulfonyl-benzamide;
3-tert-Butyl-N-(2-chloro-4-trifluoromethanesulfonyl-phenyl)-2-hydroxy-6-isopropyl-5-methylsulfanyl-benzamide;
3-tert-Butyl-N-(2-chloro-4-trifluoromethanesulfonyl-phenyl)-2-hydroxy-6-isopropyl-5-methanesulfinyl-benzamide;
3-tert-Butyl-N-(2-chloro-4-trifluoromethanesulfonyl-phenyl)-2-hydroxy-6-isopropyl-5-methanesulfonyl-benzamide;
3-tert-Butyl-N-(2-chloro-4-trifluoromethanesulfonyl-phenyl)-2-hydroxy-6-methyl-5-(2,2,2-trifluoro-ethylsulfanyl)-benzamide;
3-tert-Butyl-N-(2-chloro-4-trifluoromethanesulfonyl-phenyl)-2-hydroxy-5-trifluoromethylsulfanyl-benzamide;
3-tert-Butyl-N-(2-chloro-4-trifluoromethanesulfonyl-phenyl)-2-hydroxy-5-isopropylsulfanyl-6-methyl-benzamide;
3-tert-Butoxy-3-tert-butyl-N-(2-chloro-4-trifluoromethanesulfonyl-phenyl)-2-hydroxybenzamide;
3-tert-Butyl-N-(2-chloro-4-trifluoromethanesulfonyl-phenyl)-6-ethyl-2-hydroxy-5-methoxy-benzamide;
N-(2-Chloro-4-trifluoromethanesulfonyl-phenyl)-2-hydroxy-3-isopropyl-6-methyl-5-methylsulfanyl-benzamide;
N-(2-Chloro-4-trifluoromethanesulfonyl-phenyl)-2-hydroxy-3-isopropyl-5-methanesulfinyl-6-methyl-benzamide;
N-(2-Chloro-4-trifluoromethanesulfonyl-phenyl)-2-hydroxy-3-isopropyl-6-methyl-5-methylsulfonyl-benzamide;
3-tert-Butyl-N-(2-methoxy-4-trifluoromethanesulfonyl-phenyl)-6-methyl-2-hydroxy-5-methylsulfanyl-benzamide;
3-tert-Butyl-N-(2-methoxy-4-trifluoromethanesulfonyl-phenyl)-6-methyl-2-hydroxy-5-methanesulfinyl-benzamide;
3-tert-Butyl-N-(2-cyano-4-trifluoromethanesulfonyl-phenyl)-6-methyl-2-hydroxy-5-methanesulfonyl-benzamide;
3-tert-Butyl-N-(2-cyano-4-trifluoromethanesulfonyl-phenyl)-6-methyl-2-hydroxy-5-fluoro-benzamide;
3-tert-Butyl-N-(2-methyl-4-trifluoromethanesulfonyl-phenyl)-6-methyl-2-hydroxy-5-fluoro-benzamide;
3-tert-Butyl-N-(2-methoxy-4-trifluoromethanesulfonyl-phenyl)-6-methyl-2-hydroxy-5-fluoro-benzamide;
3-tert-Butyl-N-(2-cyano-4-trifluoromethanesulfonyl-phenyl)-6-methyl-2-hydroxy-5-chloro-benzamide;
3-tert-Butyl-N-(2-methyl-4-trifluoromethanesulfonyl-phenyl)-6-methyl-2-hydroxy-5-chloro-benzamide;
3-tert-Butyl-N-(2-methoxy-4-trifluoromethanesulfonyl-phenyl)-6-methyl-2-hydroxy-5-chloro-benzamide;
3-tert-Butyl-N-(2-cyano-4-trifluoromethanesulfonyl-phenyl)-2-hydroxy-6-methyl-5-methylsulfanyl-benzamide;
3-tert-Butyl-2-hydroxy-6-methyl-5-methylsulfanyl-N-(2-methyl-4-trifluoromethanesulfonyl-phenyl)-benzamide;
N-(2-Bromo-4-trifluoromethanesulfonyl-phenyl)-3-tert-butyl-2-hydroxy-6-methyl-5-methylsulfanyl-benzamide;
3-tert-Butyl-N-(2-cyano-4-trifluoromethanesulfonyl-phenyl)-2-hydroxy-6-methyl-5-methylsulfinyl-benzamide;
3-tert-Butyl-2-hydroxy-6-methyl-5-methylsulfinyl-N-(2-methyl-4-trifluoromethanesulfonyl-phenyl)-benzamide;
N-(2-Bromo-4-trifluoromethanesulfonyl-phenyl)-3-tert-butyl-2-hydroxy-6-methyl-5-methylsulfinyl-benzamide;
3-tert-Butyl-N-(2-cyano-4-trifluoromethanesulfonyl-phenyl)-2-hydroxy-5-methoxy-6-methyl-benzamide;
3-tert-Butyl-2-hydroxy-5-methoxy-6-methyl-N-(2-methyl-4-trifluoromethanesulfonylphenyl)-benzamide;
N-(2-Bromo-4-trifluoromethanesulfonyl-phenyl)-3-tert-butyl-2-hydroxy-5-methoxy-6-methyl-benzamide;
N-(2-Chloro-4-trifluoromethanesulfonyl-phenyl)-3-tert-butyl-2-hydroxy-5-methoxy-6-methyl-benzamide;
1-Hydroxy-3-methyl-4-methylsulfanyl-naphthalene-2-carboxylic acid (2-chloro-4-trifluoromethanesulfonyl-phenyl)-amide;
1-Hydroxy-4-methanesulfinyl-3-methyl-naphthalene-2-carboxylic acid (2-chloro-4-trifluoromethanesulfonyl-phenyl)-amide;
1-Hydroxy-4-methanesulfonyl-3-methyl-naphthalene-2-carboxylic acid (2-chloro-4-trifluoromethanesulfonyl-phenyl)-amide;
4-Chloro-1-hydroxy-3-methyl-naphthalene-2-carboxylic acid (2-chloro-4-trifluoromethanesulfonyl-phenyl)-amide;
3-tert-Butyl-N-(2-chloro-4-trifluoromethanesulfonyl-phenyl)-2-hydroxy-6-methoxy-benzamide;
6-tert-Butyl-5-hydroxy-2,3-dihydro-benzofuran-4-carboxylic acid (2-chloro-4-trifluoromethane-sulfonyl-phenyl)-amide;
3-tert-Butyl-N-(2-chloro-4-trifluoromethanesulfonyl-phenyl)-2,5-dihydroxy-6-methylbenzamide;
3-tert-Butyl-N-(2-chloro-4-trifluoromethanesulfonyl-phenyl)-2,5-dihydroxy-6-methoxy-benzamide;
3-tert-Butyl-N-(2-chloro-4-trifluoromethanesulfonyl-phenyl)-2,6-dihydroxy-5-methoxy-benzamide;
3,5-Di-tert-butyl-N-(2-chloro-4-trifluoromethanesulfonyl-phenyl)-2,6-dihydroxy-benzamide;
3-tert-Butyl-N-(2-chloro-4-trifluoromethanesulfonyl-phenyl)-5-cyano-2-hydroxy-6-methyl-benzamide;
3-tert-Butyl-N-(2-chloro-4-trifluoromethanesulfonyl-phenyl)-2-hydroxy-6-methyl-5-(4-methyl-piperazine-1-sulfonyl)-benzamide;
N-(2-Bromo-4-trifluoromethanesulfonyl-phenyl)-3-tert-butyl-5-fluoro-2-hydroxy-6-methyl-benzamide;
3-Acetyl-5-tert-butyl-N-(2-chloro-4-trifluoromethanesulfonyl-phenyl)-6-hydroxy-2-methyl-benzamide;
3-Acetyl-5-tert-butyl-6-hydroxy-N-(2-methoxy-4-trifluoromethanesulfonyl-phenyl)-2-methyl-benzamide;
3-tert-Butyl-N-(2-chloro-4-trifluoromethanesulfonyl-phenyl)-2-hydroxy-6-methyl-5-nitro-benzamide;
3-tert-Butyl-N-(2-chloro-4-trifluoromethanesulfonyl-phenyl)-2-hydroxy-6-methyl-5-(toluene-4-sulfonylamino)-benzamide;
3-Amino-5-tert-butyl-N-(2-chloro-4-trifluoromethanesulfonyl-phenyl)-6-hydroxy-2-methyl-benzamide;
3-Acetylamino-5-tert-butyl-N-(2-chloro-4-trifluoromethanesulfonyl-phenyl)-6-hydroxy-2-methyl-benzamide;
1-Hydroxy-4-(4-methoxyphenyl)naphthalene-2-carboxylic acid (2-methyl-4-trifluoromethanesulfonylphenyl)amide;
4-(4-Fluorophenyl)-1-hydroxynaphthalene-2-carboxylic acid (4-trifluoromethanesulfonylphenyl)amide;
4-(4-Fluorophenyl)-1-hydroxynaphthalene-2-carboxylic acid (2-methyl-4-trifluoromethanesulfonylphenyl)amide;
1-Hydroxynaphthalene-2-carboxylic acid (2-chloro-4-trifluoromethanesulfonylphenyl)amide;
1-Hydroxy-8-methylnaphthalene-2-carboxylic acid (2-chloro-4-trifluoromethanesulfonylphenyl)amide;
N-(3-Chloro-5-trifluoromethanesulfonyl-phenyl)-2-hydroxy-benzamide;
3-Benzylsulfanyl-5-tert-butyl-N-(2-chloro-4-trifluoromethanesulfonyl-phenyl)-6-hydroxy-2-methyl-benzamide;
3-tert-Butyl-N-(2-chloro-4-trifluoromethanesulfonyl-phenyl)-2-hydroxy-6-methyl-5-propylsulfanyl-benzamide;
3-tert-Butyl-N-(2-chloro-4-trifluoromethanesulfonyl-phenyl)-2-hydroxy-6-methyl-5-(propane-1-sulfinyl)-benzamide;
3-tert-Butyl-N-(2-chloro-4-trifluoromethanesulfonyl-phenyl)-2-hydroxy-6-methyl-5-phenylmethanesulfinyl-benzamide;
3-tert-Butyl-5-chloro-2-hydroxy-6-methyl-N-(2-propyl-4-trifluoromethanesulfonylphenyl)-benzamide;
3-tert-Butyl-N-(2-chloro-4-trifluoromethanesulfonyl-phenyl)-2-hydroxy-5-isobutylsulfanyl-6-methyl-benzamide;
3-tert-Butyl-5-chloro-N-(2-ethyl-4-trifluoromethanesulfonyl-phenyl)-2-hydroxy-6-methyl-benzamide;
3-tert-Butyl-N-(2-chloro-4-trifluoromethanesulfonyl-phenyl)-2-hydroxy-6-methyl-5-phenylmethanesulfonyl-benzamide;
3-tert-Butyl-N-(2-chloro-4-trifluoromethanesulfonyl-phenyl)-2-hydroxy-6-methyl-5-(propane-1-sulfonyl)-benzamide;
5-tert-Butyl-4′-cyano-4-hydroxy-2-methyl-biphenyl-3-carboxylic acid (2-methyl-4-trifluoromethanesulfonyl-phenyl)-amide;
5-tert-Butyl-4′-cyano-4-hydroxy-2-methyl-biphenyl-3-carboxylic acid (2-ethyl-4-trifluoromethanesulfonyl-phenyl)-amide;
5-tert-Butyl-4′-cyano-4-hydroxy-2-methyl-biphenyl-3-carboxylic acid (2-cyano-4-trifluoromethanesulfonyl-phenyl)-amide;
3-tert-Butyl-N-(2-chloro-4-trifluoromethanesulfonyl-phenyl)-2-hydroxy-6-methyl-5-(propane-2-sulfonyl)-benzamide;
3-tert-Butyl-N-(2-chloro-4-trifluoromethanesulfonyl-phenyl)-2-hydroxy-6-methyl-5-(propane-2-sulfinyl)-benzamide;
5-tert-Butyl-4′-cyano-4-hydroxy-2-methyl-biphenyl-3-carboxylic acid (2-propyl-4-trifluoromethanesulfonyl-phenyl)-amide;
3-tert-Butyl-5-chloro-N-(3-cyano-4-trifluoromethanesulfonyl-phenyl)-2-hydroxy-6-methyl-benzamide;
3-tert-Butyl-N-(2-chloro-4-trifluoromethanesulfonyl-phenyl)-2-hydroxy-6-methyl-5-(2-methyl-propane-1-sulfinyl)-benzamide;
N-(2-Chloro-4-trifluoromethanesulfonylphenyl)-2-hydroxy-3-isopropyl-6-methylbenzamide;
N-(2-Chloro-4-trifluoromethanesulfonylphenyl)-2-hydroxy-6-methylbenzamide; —Chloro-N-(2-chloro-4-trifluoromethanesulfonylphenyl)-2-hydroxybenzamide;
N-(2-Chloro-4-trifluoromethanesulfonylphenyl)-2-hydroxy-6-isopropyl-3-methylbenzamide;
N-(2-Chloro-4-trifluoromethanesulfonylphenyl)-5-fluoro-2-hydroxybenzamide;
3-Chloro-N-(2-chloro-4-trifluoromethanesulfonylphenyl)-6-hydroxy-5-isopropyl-2-methylbenzamide;
3-Chloro-N-(2-chloro-4-trifluoromethanesulfonylphenyl)-6-hydroxy-2-methylbenzamide;
3,5-Dichloro-N-(2-chloro-4-trifluoromethanesulfonylphenyl)-2-hydroxy-6-methylbenzamide;
3-Chloro-N-(2-chloro-4-trifluoromethanesulfonylphenyl)-6-hydroxy-2-isopropyl-5-methylbenzamide;
N-(2-Chloro-4-trifluoromethanesulfonylphenyl)-2-hydroxy-3,4,6-trimethylbenzamide;
4-Chloro-N-(2-chloro-4-trifluoromethanesulfonylphenyl)-2-hydroxybenzamide;
2-Hydroxybiphenyl-3-carboxylic acid (2-chloro-4-trifluoromethanesulfonylphenyl)amide;
N-(2-Chloro-4-trifluoromethanesulfonylphenyl)-2,3,4,5-tetrafluoro-6-hydroxybenzamide;
5-tert-Butyl-4-hydroxy-2-methylbiphenyl-3-carboxylic acid (2-chloro-4-trifluoromethane-sulfonylphenyl)amide;
N-(2-Chloro-4-trifluoromethanesulfonylphenyl)-3,5-difluoro-2-hydroxybenzamide;
3′,5′-Difluoro-4-hydroxy-2-methylbiphenyl-3-carboxylic acid (2-chloro-4-trifluoromethane-sulfonylphenyl)amide;
4′-Cyano-4-hydroxy-2-methylbiphenyl-3-carboxylic acid (2-chloro-4-trifluoromethanesulfonylphenyl)amide;
N-(2-Chloro-4-trifluoromethanesulfonylphenyl)-4-fluoro-2-hydroxybenzamide;
3′,5′-Difluoro-4-hydroxy-5-isopropyl-2-methylbiphenyl-3-carboxylic acid (2-chloro-4-trifluoromethanesulfonylphenyl)amide;
3-tert-Butyl-6-chloro-N-(2-chloro-4-trifluoromethanesulfonylphenyl)-2-hydroxybenzamide;
3-tert-Butyl-6-chloro-2-hydroxy-N-(2-methyl-4-trifluoromethanesulfonylphenyl)benzamide;
5-Chloro-2-hydroxy-4-methylbiphenyl-3-carboxylic acid (2-chloro-4-trifluoromethanesulfonylphenyl)amide;
5-Chloro-4′-fluoro-2-hydroxy-4-methylbiphenyl-3-carboxylic acid (2-chloro-4-trifluoromethane-sulfonylphenyl)amide;
N-(2-Chloro-4-trifluoromethanesulfonyl-phenyl)-6-hydroxy-2,4-dimethyl-3-methylsulfanyl-benzamide;
N-(2-Chloro-4-trifluoromethanesulfonyl-phenyl)-6-hydroxy-3-methanesulfinyl-2,4-dimethyl-benzamide;
3-tert-Butyl-5-tert-butylsulfanyl-N-(2-chloro-4-trifluoromethanesulfonyl-phenyl)-2-hydroxy-6-methyl-benzamide;
3-tert-Butyl-5-tert-butylsulfinyl-N-(2-chloro-4-trifluoromethanesulfonyl-phenyl)-2-hydroxy-6-methyl-benzamide;
3-tert-Butyl-5-tert-butylsulfonyl-N-(2-chloro-4-trifluoromethanesulfonyl-phenyl)-2-hydroxy-6-methyl-benzamide;
N-(2-Chloro-4-trifluoromethanesulfonyl-phenyl)-2,3-difluoro-6-hydroxy-5-methylbenzamide;
4-(4-Dimethylamino-phenyl)-1-hydroxy-naphthalene-2-carboxylic acid (2-chloro-4-trifluoromethanesulfonyl-phenyl)-amide;
2,5-Di-tert-butyl-N-(2-chloro-4-trifluoromethanesulfonyl-phenyl)-6-hydroxy-3-methoxy-benzamide;
6-tert-Butyl-2-[(2-chloro-4-trifluoromethanesulfonyl-phenylamino)-hydroxy-methyl]-3,4-dimethoxy-phenol;
3-tert-Butyl-N-(2-chloro-4-trifluoromethanesulfonyl-phenyl)-2,6-dihydroxy-benzamide; and
3-tert-Butyl-N-(2-chloro-4-trifluoromethanesulfonyl-phenyl)-2-hydroxy-6-isopropyl-5-methoxy-benzamide.

29. A pharmaceutical composition comprising a compound according to claim 1.

30. (canceled)

31. (canceled)

32. A method for: treatment of obesity per se; prevention of weight gain; treatment of diseases or disorders where obesity is involved in the etiology; or treatment of metabolic syndrome, insulin resistance, dyslipidemia, hypertension, type 2 diabetes, type 1 diabetes, diabetic late complications including cardiovascular diseases, cardiovascular disorders, disorders of lipid metabolism, neurodegenerative and psychiatric disorders, dysregulation of intraocular pressure including glaucoma, atherosclerosis, hypertension, coronary heart disease, gallbladder disease, osteoarthritis or cancer; said method comprising administering to a subject in need of such treatment, a therapeutically effective amount of a compound according to claim 1, optionally in combination with one or more additional therapeutically active compounds.

33. A method according to claim 32, wherein said additional therapeutically active compound is selected from antidiabetic agents, antihyperlipidemic agents, antiobesity agents, antihypertensive agents and agents for the treatment of complications resulting from, or associated with, diabetes.

34. A method according to claim 32, wherein obesity per se is treated.
